SEQUENCE LISTING (2 sequences)

SEQ ID NO: 1 - DNA, 1308 bp, Arabidopsis thaliana
  This is a cDNA sequence derived from mRNA encoding the putative AP2 domain transcription factor RAV1 (locus tag Atlg13260). The RAV1 coding sequence consists of nucleotides 92 through 1126 as numbered below.
gtatacatatacacaacataattcacaacacaacacaaacacatttctgttttctccattgtttcaaaccataaaaaaaaacacagattaaatggaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgtaacaactcttcttcttttttttttcttttgttgttttaataatttttaaaaactccattttcgttttctttatttgcatcggtttctttcttcttgtttaccaaaggttcatgagttgtttttgttgtattgatgaactgtaaattttatttataggataaattttaaaaagggttacttagat

SEQ ID NO: 2 - DNA, 816 bp, Arabidopsis thaliana
  This is a cDNA sequence derived from mRNA encoding the putative zinc- finger protein Zat12 (locus tag At5g59820).  The Zat12 coding sequence consists of nucleotides 102 through 590 as numbered below.
atcatcacaactactatcacaccaaactcaaaaaacacaaaccacaagaggatcatttcattttttattgtttcgttttaatcatcatcatcagaagaaaaatggttgcgatatcggagatcaagtcgacggtggatgtcacggcggcgaattgtttgatgcttttatctagagttggacaagaaaacgttgacggtggcgatcaaaaacgcgttttcacatgtaaaacgtgtttgaagcagtttcattcgttccaagccttaggaggtcaccgtgcgagtcacaagaagcctaacaacgacgctttgtcgtctggattgatgaagaaggtgaaaacgtcgtcgcatccttgtcccatatgtggagtggagtttccgatgggacaagctttgggaggacacatgaggagacacaggaacgagagtggggctgctggtggcgcgttggttacacgcgctttgttgccggagcccacggtgactacgttgaagaaatctagcagtgggaagagagtggcttgtttggatctgagtctagggatggtggacaatttgaatctcaagttggagcttggaagaacagtttattgattttatttattttccttaaattttctgaatatatttgtttctctcattctttgaatttttcttaatattctagattatacatacatccgcagatttaggaaactttcatagagtgtaatcttttctttctgtaaaaatatattttacttgtagcattggagatttgttatgagattatcttacttagcatttagtgaataatctattagcctattttgccgacgtg

(19) United States
(12) Patent Application Publication
(10) Pub. No.: US 2004/0078852 A1
(43) Pub. Date: Apr. 22, 2004

(54) Transcription factors to improve plant stress tolerance

(76) Inventors: Michael F. Thomashow, East Lansing, MI (US); Sarah George Fowler, Ann Arbor, MI (US); Jonathan Vogel, Holt, MI (US); Daniel Zarka, Lansing, MI (US)

Correspondence Address: FOSTER, SWIFT, COLLINS & SMITH, P.C., 313 SOUTH WASHINGTON SQUARE, LANSING, MI, 48933, US

(21) Appl. No.: 10/632,436

(22) Filed: Aug. 1, 2003

Related U.S. Application Data
(60) Provisional application No. 60/400,777, filed on Aug. 2, 2002.

(51) Int. Cl. A01H 1/00; C12N 9/22; C07H 21/04; C12N 15/82; C12N 5/04

(52) U.S. Cl. 800/289; 435/419; 435/199; 536/23.2

(57) ABSTRACT

DNA transcription regulating proteins capable of binding to a DNA regulatory sequence that regulates expression of one or more environmental stress tolerance genes in a plant such as to cold and drought are provided. Cold-regulated transcription factors may include ZAT12 and RAV1. The present invention relates to the DNA regulatory sequence to enhance the stress tolerance of recombinant plants into which these genes are introduced. Methods for using the DNA and transcription regulating proteins to regulate expression of one or more native or non-native environmental stress tolerance genes in a plant are provided. Use of a promoter capable of effecting mRNA transcription in the selected plant cell to be transformed may also included. Other embodiments include the use of vectors.

### CROSS-REFERENCE TO RELATED APPLICATIONS

This application claims priority to a U.S. Provisional Application Serial No. 60/400,777 titled, “Transcription Factors to Improve Plant Stress Tolerance,” filed Aug. 2, 2002. The entire disclosure of Serial No. 60/400,777 is incorporated hereby by reference in its entirety.

### GOVERNMENT RIGHTS

This invention was made with U.S. Government support under Contract No. DBI 0110124 awarded by the National Science Foundation and Contract No. DEFG0291ER20021 awarded by the U.S. Department of Energy. The U.S. Government has certain rights in this invention. This research is also supported by the Michigan Agricultural Experiment Station.

### REFERENCE TO SEQUENCE LISTING

The material contained in the Sequence Listing attached hereto and also provided on compact disc is incorporated by reference herein. The compact disc contains the following file:

Seqlist.txt 4,000 bytes created Aug. 1, 2003.

### FIELD OF INVENTION

The present invention relates to a regulatory response of plants to environmental stresses such as cold and drought. More specifically, the present invention relates to genes that regulate plant response to environmental stresses such as cold or drought and their use to enhance the stress tolerance of recombinant plants into which these genes are introduced.

### BACKGROUND OF THE INVENTION

Environmental factors serve as cues to trigger a number of specific changes in plant growth and development. One such factor is low temperature. Prominent examples of cold-regulated processes include cold acclimation, the increase in freezing tolerance that occurs in response to low non-freezing temperatures (Guy, C. L., Annu. Rev. Plant Physiol. Plant Mol. Biol. 41:187-223 (1990)); vernalization, the shortening of time to flowering induced by low temperature (Lang, A., in Encyclopedia of Plant Physiology, Vol.15-1, ed. Ruhland, W. (Springer, Berlin), pp. 1489-1536 (1965)); and stratification, the breaking of seed dormancy by low temperature (Berry, J. A. and J. K. Raison, in Encyclopedia of Plant Physiology, Vol. 12A, eds. Lange, O. L., Nobel, P. S., Osmond, C. B. and Ziegler, H. (Springer, Berlin), pp. 277-338 (1981)).

Due to the fundamental nature and agronomic importance of these processes, there is interest in understanding how plants sense and respond to low temperature. One approach is to determine the signal transduction pathways and regulatory mechanisms involved in cold-regulated gene expression.

Strong evidence exists for calcium having a role in low temperature signal transduction and regulation of at least some cold-regulated (COR) genes. Dhindsa and colleagues (Monroy, A. F., et al, Plant Physiol. 102:1227-1235 (1993); Monroy, A. F., and R. S., The Plant Cell, 7:321-331 (1995)) have shown that, in alfalfa, calcium chelators and calcium channel blockers prevent low temperature induction of COR genes and that calcium ionophores and calcium channel agonists induce expression of COR genes at normal growth temperatures. Similarly, Knight et al (The Plant Cell 8:489-503 (1996)) have shown cold-induced expression of the Arabidopsis thaliana COR gene KIN1 is inhibited by calcium chelators and calcium channel blockers (Arabidopsis thaliana is a small plant often used as a model organism in plant biology). These results suggest low temperature triggers an influx of extracellular calcium that activates a signal transduction pathway that induces the expression of COR genes. Consistent with this is the finding that low temperature evokes transient increases in cytosolic calcium levels in plants (Knight, M. R. et al, Nature 352:524-526 (1991); Knight, H., et al., The Plant Cell 8:489-503 (1996)). Additionly, low temperatures have been shown to stimulate the activity of mechanosensitive calcium-selective cation channels in plants (Ding, J. P. and B. G. Pickard, Plant J. 3:713-720 (1993)).

Recent efforts have led to the identification of a cis-acting cold-regulatory element in plants, the C-repeat/DRE (Yamaguchi-Shinozaki, et al., The Plant Cell 6:251-264 (1994); Baker, S. S., et al., Plant. Mol. Biol. 24:701-713 (1994); Jiang, C., et al., Plant Mol. Biol. 30:679-684 (1996)). The element, which has a 5 base pair core sequence for CCGAC, is present once to multiple times in all plant cold-regulated promoters that have been described to date; these include the promoters of the COR15a (Baker, S. S., et al, Plant. Mol. Biol. 24:701-713 (1994)), COR78/RD29A (Horvath, D. P., et al, Plant Physiol. 103:1047-1053 (1993); Yamaguchi-Shinozaki, K., et al., The Plant Cell 6:251-264 (1994)), COR6.6 (Wang, H., et al., Plant Mol. Biol. 28:605-617 (1995)) and KIN1 (Wang, H., et al, Plant Mol. Biol. 28:605-617 (1995)) genes of Arabidopsis and the BN115 gene of Brassica napus (White, T. C., et al, Plant Physiol. 106:917-928 (1994)). Deletion analysis of the Arabidopsis COR15a gene suggests the CCGAC sequence, designated the C-repeat, may be part of a cis-acting cold-regulatory element (Baker, S. S., et al., Plant Mol. Biol. 24:701-713 (1994)). This was first demonstrated by Yamaguchi-Shinozaki and Shinozaki (Yamaguchi-Shinozaki, K., et al., The Plant Cell 6:251-264 (1994)). Their findings showed two of the C-repeat sequences present in the promoter of COR78/RD29A induced cold-regulated gene expression when fused to a reporter gene. They also found these two elements stimulate transcription in response to dehydration and high salinity and thus, was designated the DRE (dehydration, low temperature and high salt regulatory element). Recent studies by Jiang et al (Jiang, C., et al., Plant Mol. Biol. 30:679-684 (1996)) indicate the C-repeats (referred to as low temperature response elements) present in the promoter of the B. napus BN115 gene also impart cold-regulated gene expression. U.S. Pat. Nos. 5,296,462 issued Mar. 22, 1994 and 5,356,816 issued Oct. 18, 1994 to Thomashow describe the genes encoding the proteins involved in cold adaptation in Arabidopsis thaliana. In particular the DNA encoding the COR15 proteins is described. These proteins are significant in promoting cold tolerance in plants.

The discovery of the Arabidopsis CBF cold-response pathway offers a recent important insight into the cold acclimation process (see Shinozaki and Yamaguchi-Shinozaki, Plant Physiol. 125, 89-93 (2000); Thomashow Plant Physiol. 125, 89-93 (2001)). The promoters of many cold- and dehydration-responsive genes in Arabidopsis have been shown to contain a DNA regulatory element, the CRT (C-repeat)/DRE (dehydration responsive element) (Baker et al., Plant Mol. Biol. 24, 701-713 (1994); Yamaguchi-Shinozaki and Shinozaki, Plant Cell 6, 251-264 (1994)) that confers both cold- and dehydration-responsive gene expression. A family of AP2-domain transcriptional activators, known as either the CBF (CRT binding factor) (Stockinger et al., Proc. Natl. Acad. Sci. USA 94, 1035-1040 (1997); Gilmour et al., Plant J. 16, 433-442 (1998)) or DREB1 (DRE binding) proteins (Liu et al., 1998; Shinwari et al., 1998), bind to the CRT/DRE element and activate transcription. Three members of the CBF/DREB1 family, CBF1, CBF2 and CBF3 or DREB1b, DREB1c, DREB1a, respectively, are induced within fifteen minutes of transferring plants to cold temperatures followed at about two hours by expression of the CBF regulon of target genes; i.e., those genes whose promoters contain the CRT/DRE regulatory element (Gilmour et al., 1998; Liu et al., Plant Cell 10, 1391-1406 (1998); Shinwari et al., Biochem. Biophys. Res. Comm. 250, 161-170 (1998)). The CBF regulon includes genes that act in concert to improve freezing tolerance. Overexpression of the CBF/DREB1 transcription factors in transgenic Arabidopsis plants results in the accumulation of compatible solutes that have cryoprotective activities including proline, sucrose and raffinose (Gilmour et al., Plant Physiol. 124, 1854-1865 (2000)). Additionally, the CBF regulon includes the COR15a gene and others that encode LEA or LEA-like hydrophilic polypeptides thought to have roles in freezing tolerance. Overexpression of the CBF/DREB1 proteins in Arabidopsis results in an increase in freezing tolerance at the whole plant level in both nonacclimated and cold-acclimated plants (Jaglo-Ottosen et al., Science 280, 104-106 (1998); Liu et al., 1998; Kasuga et al., Nat. Biotechnol. 17, 287-291 (1999); Gilmour et al. 2000) and enhances the tolerance of plants to dehydration caused either by imposed water deficit or exposure to high salinity (Liu et al., 1998; Kasuga et al., 1999). Recent studies (Jaglo et al., Plant Physiol. 127, 910-917 (2001)) indicate the CBF cold-response pathway is conserved in Brassica napus and components of the pathway are present in wheat and rye, which cold acclimate, as well as tomato, which does not. U.S. Pat. Nos. 5,891,859, 5,892009, 5929,305, 5,965,705, and 6,417,428 all to Thomashow et al describe CBF and the use of CBF in plants to enhance environmental stress tolerance.

Although the use of CBF in plants to enhance environmental stress tolerance is known, a need exists for the identification of additional genes that regulate expression of cold tolerance genes and drought tolerance genes. A further need exists for additional DNA constructs that are useful for introducing these regulatory genes into a plant, causing the plant to begin expressing or enhancing its expression of native or non-native cold tolerance genes and drought tolerance genes. These and other needs are provided by the present invention.

### SUMMARY OF THE INVENTION

The present invention relates to DNA transcription regulating proteins capable of binding to a DNA regulatory sequence that regulates expression of one or more environmental stress tolerance genes in a plant. The present invention also relates to the binding proteins encoded by the DNA. Methods for using the DNA and transcription regulating proteins to regulate expression of one or more native or non-native environmental stress tolerance genes in a plant are provided.

The present invention also relates to recombinant cells, plants and plant materials (e.g., plant tissue, seeds) into which one or more gene sequences that encode a transcription regulating protein have been introduced as well as cells, plants and plant materials within which recombinant binding proteins encoded by these gene sequences are expressed.

Regulation of expression may include causing one or more stress tolerance genes to be expressed under different conditions than those genes would be in the plant's native state, increasing a level of expression of one or more stress tolerance genes, and/or causing the expression of one or more stress tolerance genes to be inducible by an exogenous agent.

In the embodiments of the present invention, a plant material transformation is provided with DNA encoding a binding protein comprising an AP2 domain amino acid sequence as set forth in SEQ. ID. No. 1 and/or SEQ. ID. No. 2.

A chimeric plant-expressible gene is also provided in the 5′ to 3′ direction having a promoter capable of effecting mRNA transcription in the selected plant cell to be transformed, operably linked to a structural DNA sequence encoding SEQ. ID. No. 1 that induces freezing tolerance, operably linked to a non-translated region of a gene, said region encoding a signal sequence for polyadenylation of mRNA. Other chimeric plant-expressible gene in the 5′ to 3′ direction may include a promoter that is capable of effecting mRNA transcription in the selected plant cell to be transformed, operably linked to a structural DNA sequence encoding SEQ. ID. No. 2 that induces freezing tolerance, operably linked to a non-translated region of a gene, said region encoding a signal sequence for polyadenylation of mRNA. Drought tolerance is addressed by and linked to a non-translated region of a gene, such as encoding a signal sequence for polyadenylation of mRNA.

Other embodiments include plant tissue having plant cells susceptible to infection with Agrobacierieim tumefaciens that contain and express a chimeric genes of the present invention.

The present invention also includes methods for regulating cold and dehydration regulatory genes in a plant comprising the steps of introducing at least one copy of a regulatory gene encoding a protein into a plant; expressing the binding protein encoded by the regulatory gene; and using the expressed binding protein to stimulate expression of at least one environmental stress tolerance gene through binding to a DNA regulatory sequence.

Other methods include regulating cold and dehydration regulatory genes in a plant by transforming a plant with a gene encoding a transcription regulating protein comprising an amino acid sequence sufficiently homologous to SEQ. ID. No. 2 that the protein is capable of selectively binding to a DNA regulatory sequence in the plant which regulates expression of one or more environmental stress tolerance genes in the plant; and expressing the transcription regulating protein in the plant.

Still, other methods include regulating cold and dehydration regulatory genes in a plant by introducing DNA that encodes a binding protein capable of binding to a DNA regulatory sequence into a plant; introducing a promoter into a plant which regulates expression of the binding protein; introducing a DNA regulatory sequence into a plant to which a binding protein can bind; and introducing one or more environmental stress tolerance genes into a plant whose expression is regulated by a DNA regulatory sequence.

An additional method of the present invention includes regulating cold and dehydration regulatory genes in a plant by transforming a plant with a gene encoding a transcription regulating protein comprising an amino acid sequence sufficiently homologous to SEQ. ID. No. 1 that the protein is capable of selectively binding to a DNA regulatory sequence comprising CAACA in the plant which regulates expression of one or more environmental stress tolerance genes in the plant; and expressing the transcription regulating protein in the plant.

### BRIEF DESCRIPTION OF THE DRAWINGS

The present invention will become more fully understood from the detailed description and the accompanying drawings, wherein:

FIG. 1 is the number of GeneChip probe sets representing genes that were either up- or down-regulated at various times after transferring plants from warm (22° C.) to cold (4° C.) temperature.

FIG. 2 is the total number of up-regulated genes listed, 218, is less than 156 (transient)+64 (long-term) because probe sets representing two genes were present in both the transient and long-term lists.

FIG. 3 is the GeneChip results for genes previously reported as up-regulated during cold acclimation. Probe sets used to calculate mean average difference values were: COR47, probe sets 13225_s_at and 15997_s_at; ERD10, probe set 15103_s_at; COR78, probe set 15611_at and COR6.6, probe sets 18699_i_at, 18700_r_at, and 18701_at. Where multiple probe sets were present that corresponded to a single gene, the mean average difference obtained for all corresponding probe sets was plotted.

FIG. 4 is the hierarchical clustering of cold-responsive genes. The fold change values for genes that were up-regulated (A) (n=241 probe sets representing 218 genes) or down-regulated (B) (n=89 probe sets representing 88 genes) during cold acclimation (see Methods) were pre-processed so that fold-change values that were associated with a difference call of “No Change” were converted to 1. The mean of the four fold-change values for each time-point was then calculated and the data clustered using a Pearson correlation. Scales indicating the shading assigned to each fold change are given to the right of each cluster.

FIG. 5 is the hierarchical clustering of cold up-regulated genes. Fold-change values that were associated with a difference call of “No Change” were converted to 1. The mean fold-change values for each time-point were then calculated and the data clustered. A scale indicating the shading assigned to each fold change is given to the right of the cluster. (A) Clustering of the 64 genes (represented by 72 probe sets) that were up-regulated by at least 2.5-fold after 7 days of cold treatment. (B) Clustering of the 156 genes (represented by 169 probe sets) that were up-regulated 3-fold at any time between 30 minutes and 24 hours but were up-regulated by less than 2.5-fold after 7 days of cold treatment also occurred.

FIG. 6. is the “binary” hierarchical clustering of long-term up-regulated genes. Data points where the signal intensity indicated the gene was present for both duplicate cold samples, there was a difference call of “increase” for all four comparisons, and the fold-increase value was greater than or equal to 2.5 for all four comparisons were assigned a value of two (hatched) while all other data points were assigned a value of one (white). The resulting data was then clustered. The probe set number and description of the genes that fall into each cluster are indicated on the right.

FIG. 7 illustrates Transcript levels for possible cold-regulated transcription factors, namely: RAV1, ZAT12 and RAP2.1. (A) Two-week old wild-type (Ws) plants grown at 22° C. were cold-treated at 4° C. and tissue was harvested after the times indicated. Total RNA was isolated and northern blots were prepared (10 μg RNA). The blots were hybridized with ³²P-labeled probes for RAV1, ZAT12 and RAP2.1. (B) Total RNA was isolated from two week old plants from transgenic lines expressing the indicated CBF genes under control of the CaMV 35S promoter or carrying the empty vector (V). Total RNA was isolated from plants grown at warm temperature and northern blots were prepared (10 μg) and hybridized with a ³²P-labeled probe for RAP2.1.

FIG. 8 is the hydropathy plots for novel COR-like proteins. The amino acid sequence predicted from the sequence of COR-like proteins was analyzed using the Kyte-Doolittle method (Kyte and Doolittle, 1982) to predict regional hydropathy of the encoded polypeptides. Values greater than zero correspond to hydrophilic regions and those less than zero correspond to hydrophobic regions. The scale at the top of each plot is in number of amino acids from the N-terminus of the polypeptide. The hydropathy profile of COR6.6 is shown for comparison.

FIG. 9 is the “Binary” hierarchical clustering of transiently up-regulated genes. Data points existed where the signal intensity indicated the gene was present for both duplicate cold samples, there was a difference call of “increase” for all four comparisons, and the fold-increase value was greater than or equal to 3 for all four comparisons were assigned a value of two (hatched) while all other data points were assigned a value of one (white). The resulting data was then clustered. The probe set number and description of the genes which fall into each cluster are indicated on the right.

FIG. 10 illustrates Venn diagrams of comparisons between cold-responsive genes and genes that are part of the CBF regulon. Sets of genes were selected using the criteria described in below. The number of genes in each set is displayed within a circle above a description of the set. Genes present in two sets are shown in the intersection of the two sets so that the sum of the numbers within a circle is the total number of genes in that set: (A) Intersection of genes up-regulated in response to low temperature with those either up-regulated by or independent of CBF over-expression; (B) Intersection of genes either transiently or long-term up-regulated in response to low temperature with those either up-regulated by or independent of CBF over-expression; (C) Intersection of genes down-regulated in response to low temperature with those either down-regulated by or independent of CBF over-expression.

FIG. 11 is the sequence of RAV1 (At1g13260 and AccessionAB 013886) and defined as “SEQ ID No.1.”

FIG. 12 is the sequence of ZAT12 (At5g59820 and Accession X 98673) and defined as “SEQ ID No.2.”

### DETAILED DESCRIPTION

The following description of the preferred embodiments are merely exemplary in nature and are in no way intended to limit the invention, its application, or uses. To aid in undestanding the present invention the following defined terms as used herein are provided below:

Definitions

“Cold stress” refers to a decrease in ambient temperature, including a decrease to freezing temperatures, which causes a plant to attempt to acclimate itself to the decreased ambient temperature.

“Dehydration stress” refers to drought, high salinity and other conditions which cause a decrease in cellular water potential in a plant.

“Transformation” refers to the process for changing the genotype of a recipient organism by the stable introduction of DNA by whatever means.

A “transgenic plant” is a plant containing DNA sequences which were introduced by transformation. Horticultural and crop plants particularly benefit from the present invention. Translation means the process whereby the genetic information in an mRNA molecule directs the order of specific amino acids during protein synthesis.

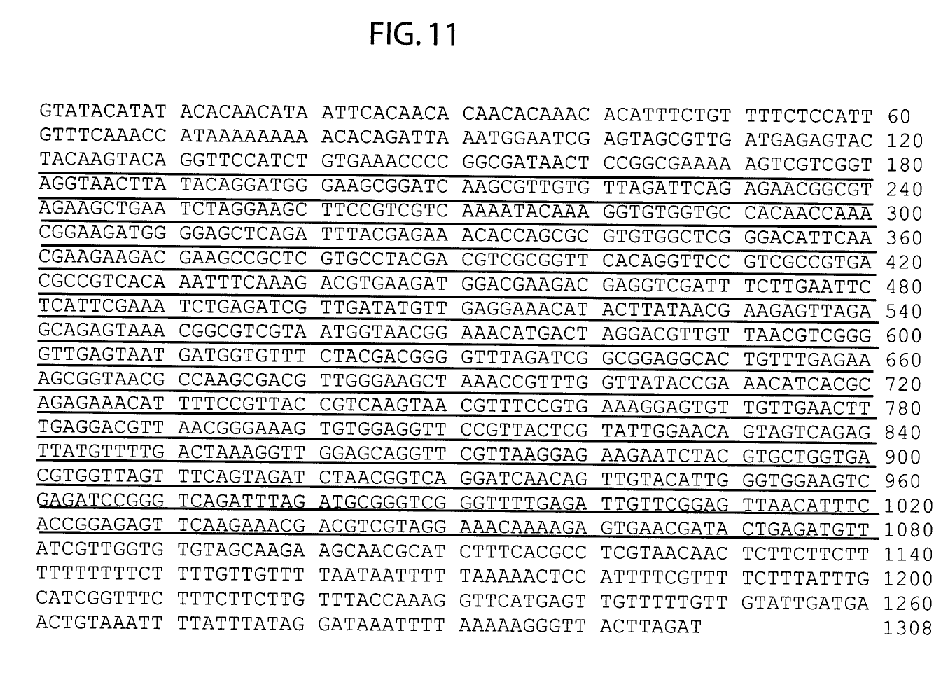

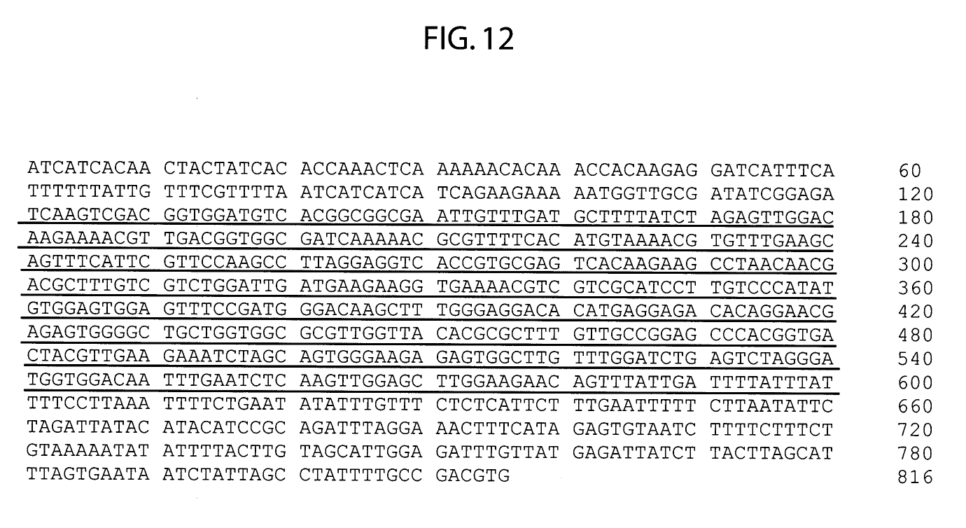

“Essentially homologous” means the DNA or protein is sufficiently duplicative of that set forth in FIG. 11 or FIG. 12 to produce the same result. Such DNA can be used as a probe to isolate DNA's in other plants.

A “promoter” is a DNA fragment that causes transcription of genetic material. For the purposes described herein, promoter is used to denote DNA fragments that permit transcription in plant cells.

A “poly-A” addition site is a nucleotide sequence that causes certain enzymes to cleave mRNA at a specific site and to add a sequence of adenylic acid residues to the 3′-end of the mRNA.

The phrase “DNA in isolated form” refers to a DNA sequence that has been at least partially separated from other DNA present in its native state in an organism. A cDNA library of genomic DNA is not “DNA in isolated form,” whereas DNA that has been at least partially purified by gel electrophoresis corresponds to “DNA in isolated form.”

The sequence of RAV1 (At1g13260 and AccessionAB 013886) is defined as “SEQ ID No.1” and illustrated in FIG. 11.

The sequence of ZAT12 (At5g59820 and AccessionX 98673) is defined as “SEQ ID No.2” and illustrated in FIG. 12.

“Transgene” refers to a heterologous gene integrated into the genome of an organism (e.g., a plant) and that is transmitted to progeny of the organism during sexual reproduction.

“Transgenic organism” refers to an organism (e.g., a plant) that has a transgene integrated into its genome and transmits the transgene to its progeny during sexual reproduction.

“Host cell” refers to any eukaryotic cell (e.g., mammalian cells, avian cells, amphibian cells, plant cells, fish cells, and insect cells), whether located in vitro or in vivo.

Where “amino acid sequence” is recited herein to refer to an amino acid sequence of a naturally occurring protein molecule, “amino acid sequence” and like terms, such as “polypeptide or protein” are not meant to limit the amino acid sequence to the complete, native amino acid sequence associated with the recited protein molecule.

The terms “nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression may be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

“Nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence.

“Recombinant DNA molecule” refers to a DNA molecule comprised of segments of DNA joined together by means of molecular biological techniques.

“Recombinant protein” or “recombinant polypeptide” refers to a protein molecule expressed from a recombinant DNA molecule.

“Transfection” refers to the introduction of foreign DNA into eukaryotic cells. Transfection may be accomplished by a variety of means known to the art including calcium phosphateDNA co-precipitation, DEAE-dextran-mediated transfection, polybrene-mediated transfection, electroporation, microinjection, liposome fusion, lipofection, protoplast fusion, retroviral infection, and biolistics.

“Vector” refers to any genetic element, such as a plasmid, phage, transposon, cosmid, chromosome, virus, virion, and the like, capable of replication when associated with the proper control elements and can transfer gene sequences between cells. Thus, the term includes cloning and expression vehicles, as well as viral vectors.

“Expression vector” refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. Nevertheless, enhancer elements may exert its effect even when located 3′ of the promoter element and the coding region. Transcription termination and polyadenylation signals are located 3′ or downstream of the coding region.

“Oligonucleotide” is defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology” and “percent identity” when used in relation to nucleic acids refers to a degree of complementarity. There may be partial homology (i.e., partial identity) or complete homology (i.e., complete identity). As used herein, a partially complementary sequence is one that at least partially inhibits a completely complementary sequence from hybridizing to a target nucleic acid sequence and is referred to using the functional term “substantially homologous.” The inhibition of hybridization of the completely complementary sequence to the target sequence may be examined using a hybridization assay (Southern or Northern blot, solution hybridization and the like) under conditions of low to high stringency. A substantially homologous sequence or probe (i.e., an oligonucleotide that is capable of hybridizing to another oligonucleotide of interest) will compete for and inhibit the binding (i.e., the hybridization) of a completely homologous sequence to a target sequence under conditions of low to high stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that may hybridize (i.e., it is the complement of) the single-stranded nucleic acid sequence under conditions of low to high stringency as described above.

“Hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

“Tₘ” is used in reference to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

“Stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent [50×DENHARDT's contains per 500 ml: 5 g Ficoll (Type 400, PHARAMCIA), 5 g BSA (Fraction V; SIGMA)] and 100 μg/ml denatured salmon sperm DNA followed by washing in a solution comprising 5×SSPE, 0.1% SDS at 42° C. when a probe of about 500 nucleotides in length is employed.

“Amplification” is a special case of nucleic acid replication involving template specificity. It is to be contrasted with non-specific template replication (i.e., replication that is template-dependent but not dependent on a specific template). Template specificity is distinguished here from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out. Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Natl. Acad. Sci. USA, 69:3038 [1972]). Other nucleic acid will not be replicated by this amplification enzyme. Similarly, in the case of T7 RNA polymerase, this amplification enzyme has a stringent specificity for its own promoters (Chamberlin et al., Nature, 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics, 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

“Primer” refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

“Polymerase chain reaction” (“PCR”) refers to the method of K. B. Mullis U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence includes introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension may be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

“PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

“Amplification reagents” refers to those reagents (deoxyribonucleotide triphosphates, buffer, and the like), needed for amplification except for primers, nucleic acid template, and the amplification enzyme. Typically, amplification reagents along with other reaction components are placed and contained in a reaction vessel (test tube, microwell, etc.).

“Reverse-transcriptase” or “RT-PCR” refers to a type of PCR where the starting material is mRNA. The starting mRNA is enzymatically converted to complementary DNA or “cDNA” using a reverse transcriptase enzyme. The cDNA is then used as a “template” for a “PCR” reaction.

“Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled nucleic acid probe (e.g., DNA or RNA) to detect DNA species complementary to the probe used. The DNA may be cleaved with restriction enzymes prior to electrophoresis and transfer to solid support. Southern blots are a standard tool of molecular biologists (Sambrook et al., Molecular Cloning: A Laboratory Manual, Cold Spring Harbor Press, NY, pp 9.31-9.58 [1989]).

“Northern blot,” refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots well known in the art (Sambrook, et al., supra, pp 7.397.52 [1989]).

“Nucleotide sequence of interest” refers to any nucleotide sequence (e.g., RNA or DNA), the manipulation of which may be desirable for any reason (e.g., treat disease, confer improved qualities, etc.) by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

“Restriction endonucleases” and “restriction enzymes” refer to bacterial enzymes, each of which cut double-stranded DNA at or near a specific nucleotide sequence.

“Recombinant DNA molecule” refers to a DNA molecule comprised of segments of DNA joined together by means of molecular biological techniques.

“Gene” refers to a nucleic acid (e.g., DNA or RNA) sequence that has coding sequences necessary for the production of an RNA, or a polypeptide or its precursor (e.g., proinsulin). A functional polypeptide may be encoded by a full length coding sequence or by any portion of the coding sequence as long as the desired activity or functional properties (e.g., enzymatic activity, ligand binding, signal transduction, etc.) of the polypeptide are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences located 5′ of the coding region and which are present on the mRNA are referred to as 5′ untranslated sequences. The sequences located 3′ or downstream of the coding region and which are present on the mRNA are referred to as 3′ untranslated sequences. The term “gene” encompasses both cDNA and genomic sequences of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Genome” refers to the genetic material (e.g., chromosomes) of an organism.

“Heterologous gene” refers to a gene encoding a factor that is not in its natural environment. For example, a heterologous gene includes a gene from one species introduced into another species. A heterologous gene also includes a gene native to an organism altered in some way (e.g., mutated, added in multiple copies, linked to non-native regulatory sequences, etc). Heterologous genes are distinguished from endogenous genes in that the heterologous gene sequences are typically joined to DNA sequences not found naturally associated with the gene sequences in the chromosome or are associated with portions of the chromosome not found in nature (e.g., genes expressed in loci where the gene is not normally expressed). The coding sequence of the heterologous gene is operatively linked to an expression control sequence. Generally a heterologous gene is first placed into a vector.

“Gene expression” refers to the process of converting genetic information encoded in a gene into RNA (e.g., mRNA, rRNA, tRNA, or snRNA) through “transcription” of the gene (i.e., via the enzymatic action of an RNA polymerase), and into protein, through “translation” of mRNA. Gene expression may be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) involved in up-regulation or down-regulation are often called “activators” and “repressors,” respectively.

“Nucleic acid molecule encoding,” “DNA sequence encoding,” “DNA encoding,” “RNA sequence encoding,” and “RNA encoding” refer to the order or sequence of deoxyribonucleotides or ribonucleotides along a strand of deoxyribonucleic acid or ribonucleic acid. The order of these deoxyribonucleotides or ribonucleotides determines the order of amino acids along the polypeptide (protein) chain. The DNA or RNA sequence thus codes for the amino acid sequence. A gene may produce multiple RNA species generated by differential splicing of the primary RNA transcript. RNA species that are splice variants of the same gene will contain regions of sequence identity or complete homology (representing the presence of the same exon or portion of the same exon on both RNAs) and regions of complete non-identity (for example, representing the presence of exon “A” on RNA 1 wherein RNA 2 contains exon “B” instead). Because the two RNAs contain regions of sequence identity both will hybridize to a probe derived from the entire gene or portions of the gene containing sequences found on both RNAs; the two splice variants are therefore substantially homologous to such a probe and to each other.

“Altered level of gene expression” as used in reference to the comparison of the level of expression of a gene in the presence and absence of a vector containing a promoter of the present invention (e.g., the LjPLP-IV promoter) refers to a measurable or observable change in the level of expression of a gene (e.g., measured through a suitable assay such as a “northern blot” or through an observable change in phenotype).

Preferred Embodiments

The present invention relates to DNA transcription regulating proteins capable of binding to a DNA regulatory sequence that regulates expression of one or more environmental stress tolerance genes in a plant. The present invention also relates to the binding proteins encoded by the DNA. The DNA and binding proteins may be native or non-native relative to the DNA regulatory sequence of the plant. The DNA and binding proteins may also be native or non-native relative to environmental stress tolerance genes of the plant which are regulated by the DNA regulatory sequence.

The present invention also relates to methods for using the DNA and transcription regulating proteins to regulate expression of one or more native or non-native environmental stress tolerance genes in a plant. These methods may include introducing DNA encoding a binding protein capable of binding to a DNA regulatory sequence into a plant, introducing a promoter into a plant that regulates expression of the binding protein, introducing a DNA regulatory sequence into a plant to which a binding protein can bind, and/or introducing one or more environmental stress tolerance genes into a plant whose expression is regulated by a DNA regulatory sequence.

The present invention also relates to recombinant cells, plants and plant materials (e.g., plant tissue, seeds) into which one or more gene sequences encoding a transcription regulating protein have been introduced as well as cells, plants and plant materials within which recombinant binding proteins encoded by these gene sequences are expressed. By introducing a gene sequence encoding a transcription regulating protein into a plant, a transcription regulating protein can be expressed within the plant which regulates expression of one or more stress tolerance genes in the plant. Regulation of expression may include causing one or more stress tolerance genes to be expressed under different conditions than those genes would be in the plant's native state, increasing a level of expression of one or more stress tolerance genes, and/or causing the expression of one or more stress tolerance genes to be inducible by an exogenous agent. Expression of the transcription regulating protein may be under the control of a variety of promoters. For example, promoters may be used to overexpress the transcription regulating protein, change the environment conditions under which the binding protein is expressed, or enable the expression of the transcription regulating protein to be induced, for example by the addition of an exogenous inducing agent.

The present invention also relates to cells, recombinant plants and plant materials into which a recombinant promoter is introduced which controls a level of expression of one or more gene sequences encoding a transcription regulating protein. The one or more gene sequences may be recombinant native or non-native sequences or may be native, non-recombinant gene sequences whose expression is altered by the introduction of the recombinant promoter.

The present invention also relates to cells, recombinant plants and plant materials into which a recombinant native or non-native DNA regulatory sequence is introduced which regulates expression of one or more native or non-native environmental stress tolerance genes.

The DNA sequence may exist in a variety of forms including a plasmid or vector and can include sequences unrelated to the gene sequence encoding the binding protein. For example, the DNA sequence can include a promoter which regulates expression of the regulatory gene.

In one embodiment of the present invention, the method includes:

taking a microorganism that includes a target DNA regulatory sequence for one or more environmental stress tolerance genes, a transcription activator for activating expression of a reporter gene, and a reporter gene whose expression is activated by a protein that includes a binding domain capable of binding to the target DNA regulatory sequence and an activation domain capable of activating the transcription activator;

fusing sequences from a cDNA library of at least a portion of a plant genome to a sequence which encodes a functional activation domain in the microorganism;

introducing fused sequences into the microorganism; and selecting microorganisms that express the reporter gene, expression of the reporter gene indicating expression of a fusion protein that includes a binding domain for the target DNA regulatory sequence and the activation domain; and identifying the gene sequence from the cDNA library introduced into the microorganism.

According to one embodiment, the protein is a recombinant transcription regulating protein expressed by a copy of a recombinant gene that is either not native to the plant or is native to the plant but introduced into the plant by recombinant methodology. For example, at least one copy of a regulatory gene may be introduced that is native to the plant but is under the control of a promoter that overexpresses the binding protein, expresses the binding protein independent of an environmental stress, expresses the binding protein at a higher level in response to the same environmental stress than would a plant in its native state, expresses the binding protein in response to different environmental stress conditions, and/or be induced to express the binding protein by an exogenous agent to which the plant can be exposed. Alternatively, at least one copy of a regulatory gene may be introduced that is not native to the plant. For example, the non-native regulatory gene may be used to alter the way in which native environmental stress tolerance genes are regulated. Alternatively, the non-native regulatory gene may be used to regulate environmental stress tolerance genes which are also not native to the plant. The non-native regulatory gene may be used to bind to a DNA regulatory region which is not native to the plant.

The present invention also relates to DNA and RNA constructs, such as plasmids, vectors, and the like, that are capable of transforming a plant. The constructs include a sequence that encodes a transcription regulating protein capable of selectively binding to a DNA regulatory sequence that regulates the one or more environmental stress tolerance genes. The binding protein is preferably able to regulate expression of one or more environmental stress tolerance genes in a plant by selectively binding to the DNA regulatory sequence. More preferably, when transformed into a plant, the sequence regulates expression of one or more environmental stress tolerance genes in the plant by expressing the binding protein. In one embodiment, the DNA construct includes a promoter and a regulatory gene sequence whose expression is under the control of the promoter. Different promoters may be used to select the degree of expression or conditions under which the regulatory gene is expressed. For example, the promoter may be used to cause overexpression of the regulatory gene, expression of the regulatory gene independent of an environmental stress, expression of the regulatory gene at a higher level in response to the same environmental stress than would a plant in its native state, expression of the regulatory gene in response to different environmental stress conditions, and/or induction of expression of the regulatory gene by an exogenous agent to which the plant can be exposed.

The present invention also relates to a recombinant microorganism, such as a bacterium, yeast, fungus, virus, into which at least one copy of a regulatory gene encoding a binding protein of the present invention has been introduced by a recombinant methodology.

The present invention also relates to recombinant plants into which at least one copy of a regulatory gene encoding a binding protein of the present invention has been introduced by a recombinant methodology. The recombinant copy of the regulatory gene may be native or non-native to the plant and express a binding protein that is either native or normative to the plant.

Expression of the recombinant copy of the regulatory gene may be under the control of the promoter. The promoter may increase the level at which the regulatory gene is expressed, express the regulatory gene without being induced by an environmental stress and/or express the regulatory gene in response to a different form or degree of environmental stress than would otherwise be needed to induce expression of the regulatory gene. For example, a promoter may be used that turns on at a temperature that is warmer than the temperature at which the plant normally exhibits cold tolerance. This would enable the cold tolerance thermostat of a plant to be altered. Similarly, a promoter may be used that turns on at a dehydration condition that is wetter than the dehydration condition at which the plant normally exhibits dehydration tolerance. This would enable the level at which a plant responds to dehydration to be altered. A promoter can also be used which causes a higher level of expression to occur at a given environmental condition (e.g. temperature and/or dryness) than the plant would express in its native state. The promoter may also be inducible by an exogenous agent, i.e., express the regulatory gene in response to the presence of an agent to which the promoter is exposed. This would enable stress tolerance to be induced by applying an inducing agent to the plant.

Selection of the promoter may also be used to determine what tissues in the plant express the binding protein as well as when expression occurs in the plant's lifecycle. By selecting a promoter that regulates in what tissues and when in a plant's life the promoter functions to regulate expression of the binding protein, in combination with the selecting how that promoter regulates expression (level of expression and/or type of environmental or chemical induction), an incredible range of control over the environmental stress responses of a plant may be achieved according to the present invention.

The environmental stress tolerance gene regulated by the recombinantly expressed regulatory gene may be native or non-native to the plant. Hence, in one embodiment, the plant includes a recombinant copy of a regulatory gene that is native to the plant and expresses a native protein that functions within the plant to regulate expression of a native environmental stress tolerance gene. In this embodiment, the recombinant plant expresses a higher level of the native regulatory gene than the plant would otherwise.

In another embodiment, at least one of the regulatory genes and the environmental stress tolerance genes is not native to the plant. For example, the regulatory gene can be native and the environmental stress tolerance gene is non-native, or the regulatory gene is non-native and the environmental stress tolerance gene is native to the plant.

In yet another embodiment, the plant may include a recombinant copy of a regulatory gene that is not native to the plant as well as a recombinant copy of one or more environmental stress tolerance genes that also is not native to the plant. According to this embodiment, the non-native regulatory gene expresses a non-native binding protein that functions within the plant to regulate expression of the one or more non-native environmental stress tolerance genes. In this regard, it is envisioned that the present invention may be used to introduce, change and/or augment the environmental stress tolerance of a plant by introducing and causing the expression of environmental stress tolerance that the plant does not have in its native form. Accordingly, plants from warmer climates may be engineered to include one or more cold tolerance genes along with a regulatory gene needed to cause expression of the cold tolerance genes in the plant so that the engineered plant may survive better in a colder climate. Similarly, a plant may be engineered to include one or more dehydration tolerance genes along with a regulatory gene needed to cause expression of the dehydration tolerance gene so that the engineered plant may grow with more vigor in a dryer climate. In this regard, it should be possible to take a plant that grows well in a first climate and engineer it to include stress tolerance genes and regulatory genes native to a second climate so that the plant can grow well in the second or non-native climate.

The present invention also relates to a method for changing or enhancing the environmental stress tolerance of a plant.

In one embodiment, the method includes introducing at least one copy of a regulatory gene encoding a binding protein of the present invention into a plant; expressing the binding protein encoded by the regulatory gene; and using the expressed binding protein to stimulate expression of at least one environmental stress tolerance gene through binding to a DNA regulatory sequence. According to this embodiment, the regulatory gene may be non-recombinant or recombinant native or non-native to the plant. Similarly, the DNA regulatory sequence and the environmental stress tolerance gene may each independently be native or non-native to the plant. In one variation of this embodiment, the method further includes recombinantly introducing an environmental stress tolerance gene into the plant that is regulated by the recombinant regulatory gene.

In another embodiment, the method includes introducing a recombinant promoter that regulates expression of a regulatory gene encoding a binding protein of the present invention into a plant; expressing the binding protein under the control of the recombinant promoter; and, using the expressed binding protein to stimulate expression of at least one environmental stress tolerance gene through binding to a DNA regulatory sequence. According to this embodiment, the regulatory gene, the DNA regulatory sequence and the environmental stress tolerance gene may each independently be non-recombinant or recombinant native or non-native to the plant.

In yet another embodiment, the method includes introducing at least one recombinant environmental stress tolerance gene into a plant; expressing a binding protein; and using the expressed binding protein to stimulate expression of the recombinant environmental stress tolerance gene through binding to a DNA regulatory sequence. According to this embodiment, the gene encoding the regulatory protein, and the DNA regulatory sequence may each independently be non-recombinant or recombinant native or non-native to the plant. The recombinant environmental stress tolerance gene may be either native or non-native to the plant.

Transcriptome Profiling Experimental Results

Central goals in cold acclimation research include identifying cold-responsive genes, determining how they are regulated, and understanding their roles in plant life at low temperature. Most studies to date have been with individual or small numbers of genes. With the development of genomic technologies, including methods for gene expression profiling, these issues may now be addressed on a broader scale. Seki et al. (Plant Cell 13, 61-72 (2001)) recently employed such methods to analyze expression of 1300 Arabidopsis genes using a cDNA micoarray. These experiments resulted in the identification of 19 cold-inducible genes, 10 of which were newly described. Nine of the 19 cold-induced genes were shown to be part of the CBF regulon i.e., they were induced in response to DREB1a expression. Moreover, 15 of the cold-inducible genes were also induced in response to drought.

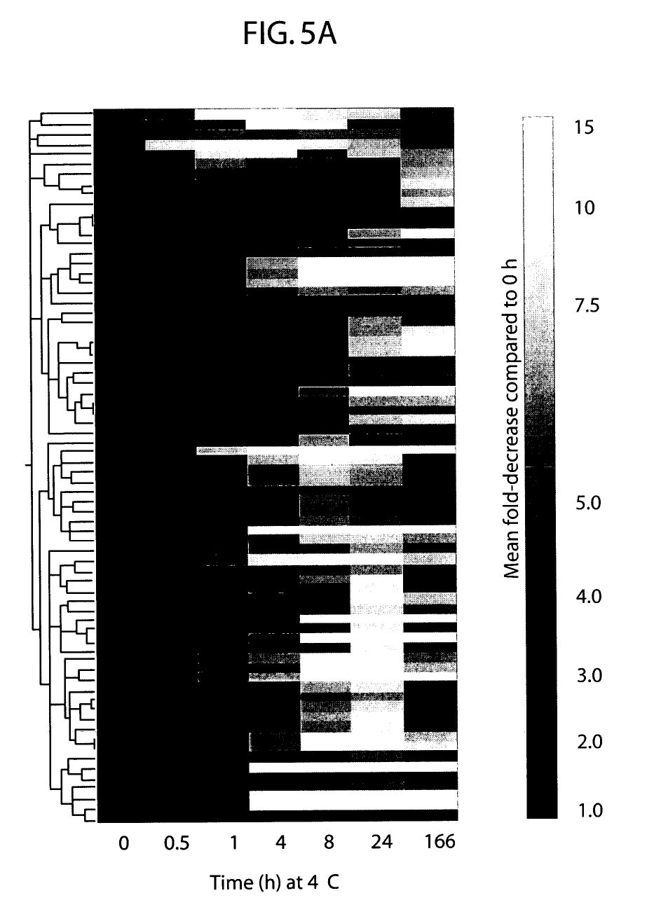

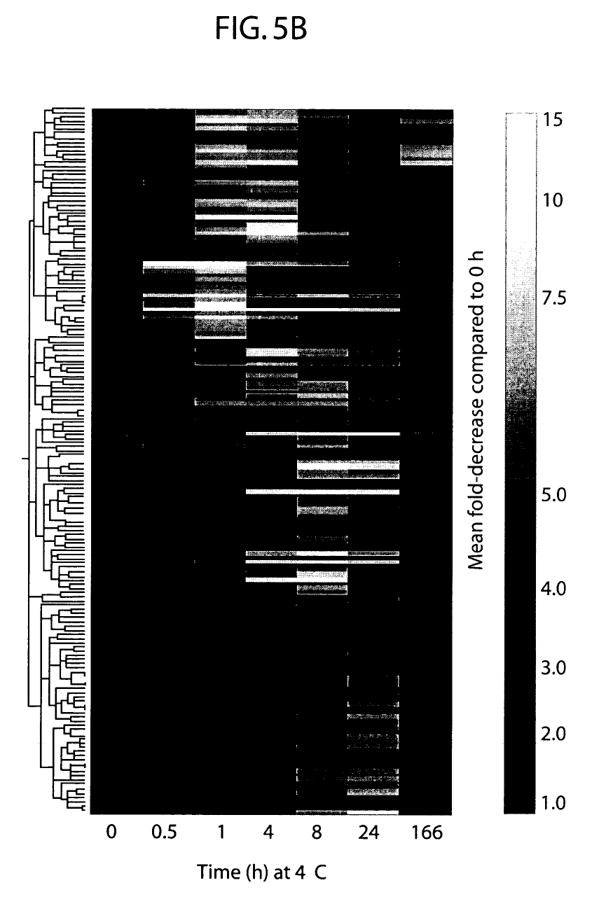

The findings of Seki et al (2001) are expanded by describing the expression of approximately 8000 genes at multiple times after transferring plants from warm to low temperature. The results provide an unprecedented view of the flux that occurs in the Arabidopsis transcriptome upon shifting plants from warm to cold temperatures. Within 30 minutes of transferring plants to low temperature, waves of changes in the composition of the transcriptome are initiated and continue to develop beyond 24 hours. A total of 306 genes were found to be affected, the large majority of which (to the best of current knowledge) have not been previously described as being cold-responsive in Arabidopsis (See FIGS. 5, 6 and 9 and Tables 1, 2 and 3). Both increases and decreases in transcript levels occurred, some of which were transient and others, long lived, being sustained up to seven days of cold treatment. The affected genes encompassed a wide range of functions including transcription, signaling, metabolism, cellular biogenesis, and cell rescue and defense.

The results indicate that expression of as much as four percent of the genome may be affected by exposing plants to low temperature. Thus, if the probe sets used in these experiments are generally representative of the entire genome, then about one thousand genes would be predicted to be cold-responsive. Still, this is probably a low estimate. Stringent criteria were used to designate a gene as being cold-responsive. Only those transcript levels that had increased or decreased at least 3-fold in each of the four comparisons of the duplicate samples were considered. Additionally, genes expressed at low levels may have been excluded. Indeed, approximately 20 percent of the probe sets gave no signal at any time-point during the experiment.

TABLE 1
Long-term up-regulated genes.
Genes up-regulated long-term by cold

| | | | | Time (h) | | | | | | |
|---|---|---|---|---|---|---|---|---|---|---|
| Probe set | AGI Identifier | Description | Sub-role | 0.5 | 1 | 4 | 8 | 24 | 168 | Peak AD |
| Metabolism | | | | | | | | | | |
| 18596_at | At1g62570 | similar to glutamate synthase | amino acid | 1.0 | 4.5 | 8.6 | 31.6 | 30.9 | 14.1 | 507.6 |
| 13018_at | At1g09350 | putative galactinol synthase | carbohydrate | 2.5 | 2.2 | 5.1 | 37.4 | 168.6 | 41.0 | 1628.1 |
| 14847_at | At1g60470 | putative galactinol synthase | carbohydrate | 1.0 | 1.0 | 1.0 | 4.5 | 9.9 | 5.3 | 109.5 |
| 13134_s_at | At2g47180 | putative galactinol synthase | carbohydrate | −1.1 | 1.0 | 1.2 | 4.2 | 5.6 | 4.0 | 522.7 |
| 18670_g_at | At4g17090 | Beta-amylase enzyme (ct-bmy) | carbohydrate | 1.1 | 1.3 | 4.4 | 7.9 | 6.5 | 3.4 | 2946.0 |
| 19421_at | At5g20830 | sucrose synthase (SUS1) | carbohydrate | 1.0 | 1.0 | 1.7 | 4.4 | 18.3 | 8.1 | 943.2 |
| 12544_at | At2g16890 | putative phenylpropanoid | secondary | 1.0 | 1.6 | 4.8 | 9.2 | 9.2 | 6.5 | 270.6 |
| | | glucosyltransferase | | | | | | | | |
| 18907_s_at | At3g51240 | flavanone 3-hydroxylase (FH3) | secondary | 1.0 | 1.7 | 3.2 | 6.5 | 5.3 | 3.8 | 648.6 |
| 20413_at | At3g55120 | chalcone isomerase | secondary | 1.0 | 1.3 | 2.4 | 4.3 | 5.1 | 5.0 | 125.6 |
| 14984_s_at | At4g27560 | UDP rhamnose-anthocyanidin-3- | secondary | 1.0 | 1.0 | 2.4 | 7.0 | 9.4 | 4.4 | 324.6 |
| | | glucoside | | | | | | | | |
| 16605_s_at | At5g08640 | flavonol synthase | secondary | 1.0 | 1.5 | 2.7 | 4.8 | 3.4 | 3.2 | 370.2 |
| Energy | | | | | | | | | | |
| 17920_s_at | At4g33070 | pyruvate decarboxylase-1 (Pdc1) | fermentation | 1.0 | 1.0 | 3.0 | 20.1 | 67.0 | 15.5 | 683.4 |
| Transcription | | | | | | | | | | |
| 15663_s_at | At1g13260 | DNA binding protein (RAV1) | mRNA synthesis | 3.5 | 5.2 | 5.1 | 5.4 | 5.3 | 3.5 | 839.2 |
| 20471_at | At1g46768 | AP2 domain protein (RAP2.1) | mRNA synthesis | 1.0 | 1.0 | 3.3 | 21.5 | 21.0 | 8.4 | 251.8 |
| 15511_s_at | At2g28550 | putative AP2 domain transcription | mRNA synthesis | 1.0 | 1.0 | 2.0 | 5.6 | 4.1 | 3.3 | 423.7 |
| | | factor (RAP2.7) | | | | | | | | |
| 16555_at | At3g47500 | H-protein promoter binding factor-2a | mRNA synthesis | 1.0 | 1.0 | 3.5 | 6.0 | 5.4 | 6.4 | 392.2 |
| 16115_at | At3g61890 | homeobox-leucine zipper protein | mRNA synthesis | 1.0 | 1.0 | 2.1 | 6.6 | 1.9 | 3.1 | 250.0 |
| | | (ATHB-12) | | | | | | | | |
| 16062_s_at | At4g25470 | AP2 domain protein (CBF2) | mRNA synthesis | 4.7 | 13.0 | 37.9 | 8.0 | 7.5 | 4.5 | 2148.9 |
| 12726_f_at | At4g37260 | R2R3-MYB transcription factor | mRNA synthesis | 3.8 | 9.2 | 10.0 | 1.0 | 8.1 | 6.0 | 92.7 |
| | | (AtMYB73) | | | | | | | | |
| 20544_at | At4g38960 | putative zinc finger protein | mRNA synthesis | 1.7 | 1.0 | 2.6 | 5.9 | 4.8 | 3.6 | 56.8 |
| 13015_s_at | At5g59820 | Zinc finger protein (ZAT12) | mRNA synthesis | 9.8 | 15.2 | 11.5 | 9.7 | 6.9 | 3.7 | 402.2 |
| 16991_at | At4g25630 | fibrillarin-like protein | mRNA processing | 1.0 | 1.6 | 1.9 | 2.3 | 5.3 | 5.1 | 262.6 |
| Protein fate | | | | | | | | | | |
| 18317_at | At1g62710 | vacuolar cysteine proteinase (beta- | targetting and | 1.0 | 1.0 | 1.0 | 1.0 | 2.0 | 3.8 | 317.6 |
| | | VPE) | sorting | | | | | | | |
| Transport | | | | | | | | | | |
| facilitation | | | | | | | | | | |
| 20149_at | At1g08890 | putative sugar transporter | carbohydrate | 1.0 | 1.0 | 2.9 | 6.9 | 11.1 | 4.6 | |
| 13950_at | At4g17550 | glycerol-3-phosphate permease like | carbohydrate | 1.1 | 1.1 | 2.9 | 8.4 | 11.4 | 4.1 | 647.7 |
| | | protein | | | | | | | | |
| Cellular | | | | | | | | | | |
| biogenesis | | | | | | | | | | |
| 19490_at | At1g10550 | xyloglucan endotransglycosylase- | cell wall | ND | 1.7 | 3.7 | 7.1 | 12.9 | 4.0 | 122.3 |
| | | related protein | | | | | | | | |
| 17963_at | At4g12470 | pEARLI 1-like protein | cell wall | 1.2 | −1.4 | 2.2 | 5.5 | 13.4 | 13.5 | 3733.2 |
| 16150_at | At4g12480 | pEARLI 1 | cell wall | −2.1 | 1.0 | 1.0 | 1.8 | 8.9 | 12.2 | 1522.7 |
| 12115_at | At4g22470 | extensin-like protein | cell wall | 1.0 | 2.1 | 3.2 | 2.3 | 4.7 | 4.4 | 43.3 |
| 15126_s_at | At2g31360 | delta 9 desaturase (ADS2) | plasma membrane | 1.0 | 1.0 | 3.0 | 3.8 | 5.8 | 3.4 | 1948.8 |
| Cellular | | | | | | | | | | |
| communication | | | | | | | | | | |
| and signal | | | | | | | | | | |
| transduction | | | | | | | | | | |
| 12395_at | At4g14580 | SNF1 like protein kinase. | intracellular | 1.3 | 2.2 | 3.9 | 3.3 | 10.4 | 4.5 | 101.6 |
| | | | signaling | | | | | | | |
| Cell rescue, | | | | | | | | | | |
| defence, cell | | | | | | | | | | |
| death and aging | | | | | | | | | | |
| 13004_at | At2g17840 | putative senescence-associated protein | aging | 1.0 | 1.5 | 7.3 | 14.3 | 9.4 | 3.1 | 644.6 |
| | | 12 | | | | | | | | |
| 18928_at | At1g52100 | putative endochitinase | defense | 1.0 | 1.0 | 1.0 | 2.3 | 10.4 | 3.2 | 433.5 |
| 12777_i_at | At1g54000 | putative myrosinase-associated protein | defense | −1.2 | 2.0 | 2.5 | 1.8 | 1.4 | 6.0 | 1371.6 |
| 16040_at | At2g02120 | protease inhibitor II | defense | 1.0 | 1.0 | 1.2 | 1.7 | 7.0 | 23.0 | 789.8 |
| 12778_r_at | At1g54000 | putative myrosinase-associated protein | defense | −1.5 | 2.5 | ND | 2.5 | 1.8 | 9.0 | 1281.3 |
| 14635_s_at | At2g14610 | PR-1-like protein | defense | −3.0 | −7.9 | −8.2 | −6.0 | 1.8 | 9.0 | 1244.8 |
| 20420_at | At4g19810 | putative chitinase | defense | 1.0 | 1.0 | 1.0 | 1.0 | 4.0 | 2.9 | 194.1 |
| 18312_s_at | At5g66390 | peroxidase (prxr8) | detoxification | 1.0 | 5.7 | 5.1 | 4.4 | 1.6 | 5.9 | 105.7 |
| 18594_at | At1g01470 | LEA protein | LEA/dehydrin | 1.0 | 1.0 | 1.5 | 8.4 | 10.6 | 4.2 | 3670.1 |
| 15997_s_at | At1g20440 | dehydrin (COR47) | LEA/dehydrin | 1.3 | 1.4 | 5.7 | 13.2 | 18.3 | 8.5 | 9751.0 |
| 13225_s_at | At1g20440 | dehydrin (COR47) | LEA/dehydrin | 1.0 | 1.6 | 5.9 | 14.5 | 19.9 | 9.7 | 4887.8 |
| 15103_s_at | At1g20450 | dehydrin (ERD10) | LEA/dehydrin | 1.1 | 2.3 | 16.4 | 38.0 | 35.1 | 19.8 | 4852.0 |
| 12749_at | At2g15970 | putative low temperature-regulated | LEA/dehydrin | 1.0 | 1.0 | 2.0 | 3.0 | 4.1 | 3.1 | 3988.5 |
| | | protein | | | | | | | | |
| 13785_at | At2g42530 | COR15b precursor | LEA/dehydrin | 1.0 | 1.0 | 18.7 | 39.7 | 54.5 | 34.1 | 2834.3 |
| 19186_s_at | At3g50970 | dehydrin (Xero 2) | LEA/dehydrin | 1.2 | 3.0 | 22.4 | 73.5 | 100.3 | 75.4 | 4765.6 |
| 18231_at | At4g15910 | Di21 | LEA/dehydrin | 1.0 | 2.2 | 2.8 | 3.0 | 8.4 | 11.1 | 256.5 |
| 16943_s_at | At4g15910 | Di21 | LEA/dehydrin | 1.0 | 1.6 | 2.3 | 2.3 | 6.4 | 9.4 | 498.2 |
| 18699_i_at | At5g15970 | COR6.6 | LEA/dehydrin | ND | 1.5 | 7.7 | 17.8 | 21.2 | 27.3 | 5687.8 |
| 18700_r_at | At5g15970 | COR6.6 | LEA/dehydrin | −1.5 | 1.4 | 5.9 | 12.5 | 17.0 | 19.4 | 4957.8 |
| 18701_s_at | At5g15970 | COR6.6 | LEA/dehydrin | −1.2 | 1.2 | 7.3 | 21.9 | 31.1 | 32.8 | 8074.9 |
| 15611_s_at | At5g52310 | COR78 | LEA/dehydrin | 1.0 | 1.0 | 25.9 | 93.5 | 152.4 | 112.4 | 4904.4 |
| Unknown role | | | | | | | | | | |
| 14298_g_at | At1g22770 | GIGANTEA | unknown | 1.0 | 1.0 | 2.1 | 3.3 | 8.0 | 9.3 | 292.0 |
| 17580_at | At1g22770 | GIGANTEA | unknown | 1.0 | 1.0 | 1.5 | 2.2 | 4.8 | 4.8 | 1520.4 |
| 17581_g_at | At1g22770 | GIGANTEA | unknown | 1.0 | 1.0 | 1.6 | 2.8 | 7.2 | 7.3 | 857.1 |
| 19189_at | At3g21490 | farnesylated protein (ATFP6). | unknown | 1.0 | 1.0 | 1.9 | 5.1 | 7.0 | 3.1 | 1846.3 |
| 16637_s_at | At3g22840 | early light-inducible protein (ELIP1) | unknown | 1.0 | 8.7 | 83.0 | 160.9 | 150.5 | 25.0 | 2215.3 |
| 13812_at | At4g03400 | putative GH3-like protein | unknown | 1.2 | 1.2 | 3.6 | 5.1 | 6.9 | 3.1 | 483.7 |
| Unknown | | | | | | | | | | |
| proteins | | | | | | | | | | |
| 19394_at | At1g14170 | unknown protein | unknown | 1.0 | 1.0 | 1.0 | 1.0 | 2.0 | 3.8 | 48.7 |
| 14367_at | At1g60190 | unknown protein | unknown | 1.0 | 2.0 | 11.5 | 10.2 | 2.4 | 3.3 | 130.5 |
| 12766_at | At2g05380 | unknown protein (COR8.6) | unknown | 1.0 | 1.0 | 1.0 | 1.0 | 1.0 | 3.8 | 2447.2 |
| 12767_at | At2g23120 | unknown protein (COR8.5) | unknown | 1.1 | 1.7 | 5.4 | 8.0 | 6.5 | 4.3 | 3884.0 |
| 11997_at | At2g24110 | unknown protein | unknown | 1.0 | 1.0 | 1.4 | 2.8 | 3.8 | 3.3 | 164.6 |
| 15046_s_at | At2g39710 | unknown protein | unknown | 1.0 | 1.0 | 1.2 | 2.7 | 2.0 | 3.4 | 190.9 |
| 20174_at | At2g43060 | unknown protein (COR18) | unknown | 1.0 | 1.0 | 1.0 | 1.0 | 3.5 | 3.5 | 139.6 |
| 14784_at | At2g46790 | hypothetical protein | unknown | 1.0 | 1.0 | 1.2 | 1.4 | 3.4 | 3.0 | 163.1 |
| 14785_g_at | At2g46790 | hypothetical protein | unknown | 1.0 | 1.0 | 1.6 | 2.7 | 6.8 | 5.0 | 73.3 |
| 20044_at | At3g21150 | putative protein | unknown | 3.3 | 4.5 | 12.2 | 14.3 | 21.3 | 8.9 | 447.4 |
| 19368_at | At3g27330 | unknown protein | unknown | 2.2 | 4.3 | 6.5 | 16.2 | 18.7 | 6.1 | 245.6 |
| 13656_at | At4g01870 | predicted protein | unknown | 1.0 | 1.9 | 4.4 | 5.0 | 12.0 | 3.8 | 914.5 |
| 15437_at | At4g33550 | putative protein (COR12) | unknown | 1.0 | 4.3 | 4.0 | 3.3 | 2.7 | 6.9 | 216.0 |
| 15878_at | At4g33980 | putative protein (COR28) | unknown | 1.0 | 1.2 | 5.5 | 4.4 | 7.7 | 4.2 | 437.6 |
| Probe set names in boldface correspond to genes that have previously been reported as up-regulated by cold treatment. AGI identifier numbers, descriptions and role assignments were derived from data provided by AFFYMETRIX, GENBANK, NCBI | | | | | | | | | | |
| #(http://www.ncbi.nlm.nih.gov/cgi-bin/Entrez/map search?chr=arabid.inf) and MIPS (http://mips.gsf.de/proj/thal/db/index.html). Mean fold-change values at each time-point are shown with fold-change values associated with a difference call of “No Change” conv 1.0. Fold-change values where the data passed the criteria for 2.5-fold up-regulation (see Methods) are in boldface. | | | | | | | | | | |
| #ND indicates where the four difference calls associated with a time-point included both “Increase” and “Decrease” calls and therefore a mean fold-change was not determined. Peak AD is the greatest of the mean average difference values obtained during the cold treatment. | | | | | | | | | | |

TABLE 2
Transiently up-regulated genes.
Genes up-regulated transiently by cold

| | | | | Time (h) | | | | | | Peak |
|---|---|---|---|---|---|---|---|---|---|---|
| Probe set | AGI Identifier | Description | Sub-role | 0.5 | 1 | 4 | 8 | 24 | 168 | AD |
| Metabolism | | | | | | | | | | |
| 14918_at | At2g32020 | putative alanine acetyl transferase | amino acid | 1.4 | 5.0 | 6.9 | 3.9 | 1.0 | 1.0 | 54 |
| 16912_at | At3g55610 | pyrroline-5-carboxlyate synthetase | amino acid | −1.1 | 1.0 | 1.0 | 2.6 | 5.3 | 1.8 | 1175 |
| | | (P5CS2) | | | | | | | | |
| 18211_s_at | At4g29510 | arginine methyltransferase (pam1) | amino acid | 1.1 | 1.1 | 1.1 | 1.7 | 3.6 | 1.4 | 244 |
| 14126_s_at | At4g34740 | amidophosphoribosyltransferase 2 | amino acid | 1.0 | 1.0 | 1.4 | 2.5 | 3.6 | 1.5 | 364 |
| | | precursor | | | | | | | | |
| 15975_s_at | At1g62660 | vacuolar invertase (AtBFRUCT3) | carbohydrate | 1.0 | 4.3 | 2.9 | 2.4 | 1.0 | 2.0 | 101 |
| 12998_at | At3g47800 | aldose 1-epimerase-like protein | carbohydrate | 1.1 | 1.0 | 1.5 | 1.5 | 3.7 | 2.4 | 221 |
| 17782_at | At4g15480 | indole-3-acetate beta- | carbohydrate | 1.0 | 1.8 | 3.2 | 5.0 | 2.0 | 1.7 | 41 |
| | | glucosyltransferase like protein | | | | | | | | |
| 17936_s_at | At4g17090 | beta-amylase (ct-bmy) | carbohydrate | 1.6 | 1.1 | 3.0 | 4.5 | 4.3 | 2.1 | 2349 |
| 18669_at | At4g17090 | beta-amylase (ct-bmy) | carbohydrate | ND | 1.6 | 3.1 | 4.4 | 3.9 | 3.2 | 2741 |
| 18955_at | At1g04220 | putative beta-ketoacyl-CoA synthase | lipid | 1.0 | 2.6 | 1.7 | 4.7 | 1.8 | 2.2 | 63 |
| 16192_at | At2g24560 | putative GDSL-motif lipase/hydrolase | lipid | 1.0 | 1.0 | 1.0 | 2.1 | 4.4 | 1.0 | 41 |
| 15186_s_at | At1g62180 | adenosine-5-phosphosulfate reductase | nitrogen and sulfur | 1.0 | 1.0 | 2.3 | 4.1 | 1.3 | 1.3 | 1301 |
| | | (APSR) | | | | | | | | |
| 18696_s_at | At1g62180 | adenosine-5-phosphosulfate reductase | nitrogen and sulfur | 1.0 | 1.0 | 2.0 | 3.5 | −1.1 | 1.0 | 1586 |
| | | (APSR) | | | | | | | | |
| 17827_at | At5g23300 | dihydroorotate dehydrogenase (pyrD) | nucleotide | 1.0 | 1.6 | 1.0 | 1.0 | 3.5 | 1.0 | 42 |
| 14016_s_at | At1g30700 | putative reticuline oxidase-like protein | secondary | 1.5 | 3.8 | 4.4 | 4.7 | 1.4 | 2.7 | 38 |
| 14797_s_at | At2g22590 | putative anthocyanidin-3-glucoside | secondary | 1.0 | 1.0 | 2.5 | 5.3 | 3.8 | 1.9 | 47 |
| | | rhamnosyltransferase | | | | | | | | |
| 16712_at | At2g35710 | putative glycogenin | secondary | 1.7 | 4.9 | 5.6 | 1.7 | 1.0 | 1.0 | 82 |
| 17392_at | At3g53260 | phenylalanine ammonia lyase (PAL2) | secondary | 1.0 | 1.1 | 2.5 | 3.5 | 2.8 | 2.7 | 416 |
| 13908_s_at | At4g20860 | putative reticuline oxidase-like protein | secondary | 1.5 | 3.3 | 4.0 | 3.9 | 1.5 | 1.8 | 222 |
| 18597_at | At4g20860 | putative reticuline oxidase-like protein | secondary | 1.4 | 2.9 | 3.8 | 3.5 | 1.4 | 1.6 | 202 |
| 19348_at | At4g26220 | caffeoyl-CoA O-methyltransferase-like | secondary | 1.0 | 4.3 | 2.1 | 1.3 | −2.2 | 2.5 | 36 |
| | | protein | | | | | | | | |
| Cell | | | | | | | | | | |
| growth, division, | | | | | | | | | | |
| DNA synthesis | | | | | | | | | | |
| 13363_s_at | At1g22730 | putative topoisomerase | DNA synthesis | 1.0 | 1.2 | 1.6 | 2.2 | 4.2 | 3.0 | 39 |
| Transcription | | | | | | | | | | |
| 13435_at | AF003102 | AP2 domain containing protein | mRNA synthesis | 1.0 | 1.0 | 3.0 | 4.7 | 4.0 | 2.0 | 140 |
| | | (RAP2.9) | | | | | | | | |
| 18745_f_at | At1g06180 | R2R3-MYB transcription factor | mRNA synthesis | 1.0 | 1.0 | 1.0 | 4.5 | 2.0 | 1.0 | 117 |
| | | (AtMYB32) | | | | | | | | |
| 18216_at | At1g27730 | salt-tolerance zinc finger protein (STZ) | mRNA synthesis | 20.3 | 36.3 | 15.3 | 12.4 | 7.0 | 2.7 | 477 |
| 18217_g_at | At1g27730 | salt-tolerance zinc finger protein (STZ) | mRNA synthesis | 20.0 | 20.1 | 7.6 | 6.2 | 3.9 | 1.6 | 1591 |
| 19672_at | At1g43160 | AP2 domain protein (RAP2.6) | mRNA synthesis | 1.0 | 2.9 | 3.1 | 4.7 | 3.2 | 2.5 | 40 |
| 19855_at | At1g78600 | putative zinc finger protein | mRNA synthesis | 1.0 | 1.4 | 4.2 | 2.4 | 3.9 | 1.0 | 304 |
| 12945_at | At2g21320 | putative CONSTANS-like B-box zinc | mRNA synthesis | 1.0 | 1.0 | 1.3 | 4.3 | 4.0 | 2.9 | 232 |
| | | finger protein | | | | | | | | |
| 12375_s_at | At2g23290 | R2R3-MYB transcription factor | mRNA synthesis | 2.6 | 3.5 | 3.9 | 1.4 | 3.9 | 2.5 | 455 |
| | | (AtMYB73) | | | | | | | | |
| 20456_at | At2g23340 | putative AP2 domain protein | mRNA synthesis | 1.0 | 1.0 | 1.2 | 5.5 | 1.6 | 1.0 | 413 |
| 18197_at | At2g23760 | putative homeodomain transcription | mRNA synthesis | 1.1 | 1.0 | 1.3 | 2.6 | 3.6 | 1.2 | 184 |
| | | factor | | | | | | | | |
| 20525_at | At2g31380 | putative CONSTANS-like B-box zinc | mRNA synthesis | 1.0 | 1.0 | 3.8 | 6.3 | 3.2 | 2.0 | 1660 |
| | | finger protein | | | | | | | | |
| 17303_s_at | At2g38470 | WRKY domain protein (AtWRKY33) | mRNA synthesis | 6.0 | 8.4 | 3.6 | −1.5 | 1.0 | 1.0 | 609 |
| 14711_at | At2g40140 | hypothetical Cys-3-His zinc finger | mRNA synthesis | 4.6 | 6.6 | 3.7 | 2.6 | 2.4 | 1.6 | 358 |
| | | protein | | | | | | | | |
| 17379_at | At2g40140 | hypothetical Cys-3-His zinc finger | mRNA synthesis | 3.9 | 4.6 | 2.9 | 2.0 | 2.1 | 1.4 | 569 |
| | | protein | | | | | | | | |
| 14123_at | At2g45660 | MADS-box protein (SOC1/AGL20) | mRNA synthesis | 1.0 | 1.0 | 1.7 | 2.4 | 4.0 | 1.6 | 172 |
| 19202_at | At2g45680 | putative PCF2-like DNA-binding | mRNA synthesis | 1.5 | 2.1 | 5.9 | 6.9 | 1.8 | 1.0 | 222 |
| | | protein | | | | | | | | |
| 17019_s_at | At2g46830 | MYB-related transcription factor | mRNA synthesis | 1.0 | 1.0 | 1.4 | 3.1 | 3.3 | 1.6 | 688 |
| | | (CCA1) | | | | | | | | |
| 12505_s_at | At2g47890 | B-box zinc finger protein (COL13) | mRNA synthesis | 1.0 | 1.0 | 1.2 | 2.4 | 3.7 | 2.2 | 455 |
| 16064_s_at | At3g15210 | ethylene responsive element binding | mRNA synthesis | 1.9 | 3.3 | 3.4 | 2.9 | 1.8 | 1.0 | 468 |
| | | factor (AtERF4) | | | | | | | | |
| 13296_at | At3g15540 | auxin-responsive protein (IAA19) | mRNA synthesis | 1.0 | 1.0 | 1.0 | 1.7 | 3.9 | 1.0 | 173 |
| 20335_s_at | At3g50060 | R2R3-MYB transcription factor | mRNA synthesis | 3.8 | 5.4 | 2.9 | 1.0 | 1.0 | 1.0 | 327 |
| | | (AtMYB77) | | | | | | | | |
| 19869_at | At4g01250 | WRKY domain protein (AtWRKY22) | mRNA synthesis | 2.4 | 5.9 | 2.3 | 1.0 | 1.2 | 1.0 | 89 |
| 13289_s_at | At4g14560 | auxin-responsive protein (IAA1). | mRNA synthesis | 2.3 | 2.6 | 1.7 | 3.7 | 7.9 | 2.7 | 82 |
| 15613_s_at | At4g16780 | homeobox protein (HAT4/ATHB-2) | mRNA synthesis | 1.1 | 2.0 | 4.0 | 1.7 | 2.6 | 1.0 | 68 |
| 16539_s_at | At4g17490 | ethylene responsive element binding | mRNA synthesis | 6.6 | 14.8 | 7.3 | 1.0 | 1.0 | −1.5 | 344 |
| | | factor (AtERF6) | | | | | | | | |
| 19209_s_at | At4g18390 | putative bHLH DNA binding protein | mRNA synthesis | 1.0 | 1.0 | 1.3 | 1.9 | 3.6 | 1.4 | 425 |
| | | (TCP2) | | | | | | | | |
| 20455_at | At4g23750 | putative Ap2 domain protein | mRNA synthesis | 1.0 | 2.1 | 3.9 | 1.8 | 1.0 | −3.1 | 141 |
| 14431_at | At4g23810 | WRKY domain protein (AtWRKY53) | mRNA synthesis | 3.7 | 6.5 | −1.5 | −1.7 | 1.0 | 1.0 | 93 |
| 16111_f_at | At4g25480 | AP2 domain protein (CBF1) | mRNA synthesis | 2.6 | 12.2 | 22.5 | 2.7 | 2.6 | 2.4 | 270 |
| 17520_s_at | At4g25490 | AP2 domain protein (CBF3) | mRNA synthesis | 1.0 | 1.7 | 6.8 | 2.0 | 2.0 | 1.3 | 1720 |
| 19611_s_at | At4g34990 | putative transcription factor | mRNA synthesis | 1.0 | 1.0 | 1.0 | 4.9 | 2.7 | 1.5 | 130 |
| | | (AtMYB32) | | | | | | | | |
| 15665_at | At5g04340 | putative c2h2 zinc finger transcription | mRNA synthesis | 3.9 | 4.2 | 2.9 | 7.6 | 4.2 | 2.0 | 421 |
| | | factor | | | | | | | | |
| 20299_at | At5g15850 | B-box zinc finger protein (COL1) | mRNA synthesis | ND | 1.2 | 3.6 | 5.1 | 3.0 | 2.7 | 1361 |
| 20300_g_at | At5g15850 | B-box zinc finger protein (COL1) | mRNA synthesis | 1.0 | 1.0 | 3.6 | 5.8 | 2.8 | 2.5 | 1300 |
| 16536_at | At5g47230 | ethylene responsive element binding | mRNA synthesis | 9.8 | 26.3 | 4.3 | 7.0 | 4.3 | 1.0 | 751 |
| | | factor (AtERF5) | | | | | | | | |
| 18950_at | At5g47370 | homeobox protein (HAT2) | mRNA synthesis | 1.2 | 1.5 | 1.8 | 2.2 | 4.3 | 1.0 | 207 |
| 18949_at | At5g67300 | R2R3-MYB transcription factor | mRNA synthesis | 1.6 | 2.1 | 4.2 | 2.1 | 2.1 | 1.6 | 753 |
| | | (AtMYB44) | | | | | | | | |
| 19707_s_at | At5g67300 | R2R3-MYB transcription factor | mRNA synthesis | 1.0 | 1.4 | 4.0 | 1.9 | 1.9 | 1.0 | 679 |
| | | (AtMYB44) | | | | | | | | |
| 15872_at | At3g22310 | DEAD box RNA helicase (RH9) | mRNA processing | 1.0 | 1.0 | 1.0 | 1.5 | 5.3 | 1.0 | 160 |
| 19930_at | At5g08620 | DEAD box RNA helicase (RH25) | mRNA processing | 1.1 | 1.0 | 1.2 | 1.8 | 4.3 | 2.4 | 89 |
| Protein fate | | | | | | | | | | |
| 19322_at | At1g47710 | putative serpin | proteolysis | 1.0 | 1.0 | 1.6 | 4.7 | 6.8 | 2.5 | 526 |
| 16845_at | At2g27420 | putative cysteine proteinase | proteolysis | 1.8 | 1.8 | 5.4 | 9.0 | −1.7 | −1.7 | 139 |
| 14604_at | At3g48340 | cysteine endopeptidase-like protein | proteolysis | 1.0 | 4.9 | 2.6 | 3.2 | 1.0 | 2.2 | 50 |
| 17047_at | At4g35480 | RING-H2 finger protein (RHA3b) | proteolysis | 1.2 | 1.4 | 3.5 | 4.3 | 3.4 | 1.3 | 294 |
| Transport | | | | | | | | | | |
| facilitation | | | | | | | | | | |
| 16636_at | At5g44110 | putative ABC transporter (AtNAP2) | ABC transporters | 1.0 | 1.0 | 2.8 | 8.4 | 1.6 | 1.6 | 339 |
| 12698_at | At1g08920 | putative sugar transport protein | carbohydrate | 1.0 | 1.5 | 1.2 | 3.2 | 5.9 | 1.9 | 339 |
| | | (SUGTL2) | | | | | | | | |
| 16911_at | At3g27170 | chloride channel protein (CLC-b) | ion transporters | 1.1 | 1.0 | 1.0 | 4.4 | 1.0 | 1.0 | 209 |
| 17041_s_at | At3g51895 | sulfate transporter (ATST1) | ion transporters | −1.4 | 1.0 | 1.6 | 4.0 | 1.9 | 1.0 | 130 |
| 12773_at | At2g28900 | putative water channel protein | other | −1.2 | 1.0 | 1.0 | 2.6 | 4.6 | 2.4 | 3128 |
| 19847_s_at | At4g19030 | nodulin-26-like intrinsic protein | other | 1.5 | 10.1 | 8.2 | 3.7 | −1.3 | 5.4 | 131 |
| | | (NIP1;1) | | | | | | | | |
| Intracellular | | | | | | | | | | |
| transport | | | | | | | | | | |
| 13617_at | At2g22500 | putative mitochondrial dicarboxylate | mitochrondrial | 2.0 | 3.2 | 5.3 | 3.6 | 1.3 | 1.0 | 109 |
| | | carrier protein | | | | | | | | |
| Cellular | | | | | | | | | | |
| biogenesis | | | | | | | | | | |
| 16575_s_at | At5g40420 | oleosin. | vessicles | 1.0 | 2.3 | 4.6 | 2.2 | 1.0 | 1.0 | 44 |
| 19267_s_at | At1g02810 | putative pectin methylesterase | cell wall | 1.7 | 3.1 | 3.5 | 2.1 | 3.3 | 1.5 | 89 |
| 17960_at | At1g65310 | putative xyloglucan | cell wall | 1.2 | 4.7 | 5.3 | 5.4 | 3.8 | 1.9 | 53 |
| | | endotransglycosylase | | | | | | | | |
| 15954_at | At1g66270 | beta-glucosidase (psr3.2) | cell wall | 1.0 | 8.6 | ND | 5.0 | −3.2 | 8.8 | 332 |
| 16630_s_at | At4g25820 | putative xyloglucan | cell wall | 1.5 | 9.5 | 7.0 | 4.5 | 1.3 | ND | 175 |
| | | endotransglycosylase (XTR9) | | | | | | | | |
| 18917_i_at | At4g27820 | putative beta-glucosidase | cell wall | 1.0 | 1.0 | 1.6 | 3.4 | 3.7 | 1.8 | 300 |
| 16489_at | At5g46900 | extensin (extA) | cell wall | 1.0 | 9.7 | 13.6 | 7.4 | −1.4 | 1.3 | 182 |
| 16620_s_at | At5g57560 | xyloglucan endotransglycosylase | cell wall | 4.7 | 6.5 | 7.8 | 2.6 | 2.8 | −3.1 | 961 |
| | | related (TCH4) | | | | | | | | |
| Cellular | | | | | | | | | | |
| communication | | | | | | | | | | |
| and signal | | | | | | | | | | |
| transduction | | | | | | | | | | |
| 12891_at | At4g11280 | ACC synthase (AtACS-6) | intercellular | 3.2 | 6.6 | 3.1 | 1.0 | 1.8 | 1.0 | 216 |
| | | | communication | | | | | | | |
| 12892_g_at | At4g11280 | ACC synthase (AtACS-6) | intercellular | 5.0 | 5.6 | 2.9 | 1.0 | 1.8 | 1.0 | 255 |
| | | | communication | | | | | | | |
| 16817_s_at | At4g11280 | ACC synthase (AtACS-6) | intercellular | 5.0 | 7.1 | 3.9 | 1.4 | 1.7 | 1.0 | 208 |
| | | | communication | | | | | | | |
| 13868_at | At1g15440 | putative WD40 containing gene | intracellular signaling | 1.0 | 1.0 | 1.0 | 1.5 | 3.4 | 1.6 | 107 |
| 16610_s_at | At1g19050 | response regulator (ARR7) | intracellular signaling | 1.3 | 4.2 | 5.5 | 1.0 | −1.4 | −3.0 | 287 |
| 16840_at | At1g30640 | putative protein kinase | intracellular signaling | 1.1 | 1.3 | 1.4 | 3.2 | 2.5 | 1.8 | 31 |
| 16342_at | At1g61380 | S-like receptor protein kinase | intracellular signaling | 1.0 | 1.0 | 1.4 | 2.5 | 4.1 | 1.9 | 185 |
| 14048_at | At2g18890 | putative protein kinase | intracellular signaling | 1.0 | 1.0 | 1.0 | 3.2 | 4.4 | 1.9 | 86 |
| 19263_at | At2g30020 | putative protein phosphatase 2C | intracellular signaling | 3.4 | 7.1 | 1.6 | 1.0 | 1.0 | 1.0 | 64 |
| 15005_s_at | At2g30040 | putative protein kinase | intracellular signaling | 2.6 | 6.0 | 2.9 | 3.3 | 1.0 | 1.0 | 40 |
| 20689_at | At2g43290 | putative Ca2+-binding protein | intracellular signaling | 2.2 | 4.9 | 5.5 | 3.1 | 1.6 | 1.0 | 225 |
| 15633_s_at | At3g23000 | SNF1 related protein kinase | intracellular signaling | −1.4 | 1.0 | 2.8 | 4.7 | 2.2 | 2.0 | 1851 |
| | | (AtSRPK1) | | | | | | | | |
| 15184_s_at | At3g48100 | response regulator (ARR5) | intracellular signaling | 1.8 | 6.7 | 8.3 | 1.4 | −2.9 | −1.5 | 957 |
| 16290_at | At4g04940 | putative WD-repeat membrane protein | intracellular signaling | 1.0 | 1.0 | 1.0 | 1.0 | 4.1 | 1.4 | 45 |
| 16232_s_at | At4g08260 | putative protein phosphatase 2c | intracellular signaling | 5.9 | 13.8 | 1.6 | 1.0 | 1.0 | 1.0 | 183 |
| 15641_s_at | At4g18010 | putative inositol polyphosphate 5- | intracellular signaling | 1.0 | 2.3 | 9.6 | 6.7 | 1.8 | 1.0 | 93 |
| | | phosphatase (At5P2) | | | | | | | | |
| 18012_s_at | At4g18010 | inositol-1,4,5-trisphosphate 5- | intracellular signaling | 1.6 | 4.4 | 10.1 | 7.6 | 4.6 | 1.0 | 87 |
| | | phosphatase | | | | | | | | |
| 18300_at | At5g37770 | calmodulin-related protein (TCH2) | intracellular signaling | 3.7 | 7.3 | 3.6 | 1.5 | 1.0 | 1.0 | 187 |
| Cell rescue, | | | | | | | | | | |
| defence, cell | | | | | | | | | | |
| death and aging | | | | | | | | | | |
| 16001_at | At3g15450 | senescence associated protein (SEN5) | aging | 1.4 | 1.8 | 2.4 | 1.8 | 4.7 | 2.1 | 2212 |
| 12956_i_at | At1g05170 | putative AVR9 elicitor response | defense | 1.6 | 1.4 | 1.8 | 7.0 | 6.0 | 1.6 | 112 |
| | | protein | | | | | | | | |
| 14050_at | At1g52050 | putative myrosinase-binding protein | defense | 1.0 | 5.4 | 5.2 | 2.4 | 1.0 | 1.0 | 75 |
| 18571_at | At1g52070 | myrosinase-binding protein-like | defense | 1.0 | 6.4 | 10.0 | 3.9 | 1.0 | 1.9 | 85 |
| 19577_at | At1g65390 | RPS4-like protein | defense | 1.4 | 2.7 | 4.3 | 1.8 | 1.0 | 1.0 | 32 |
| 12251_at | At2g34930 | putative disease resistance protein | defense | 1.7 | 3.9 | 10.2 | 12.7 | 6.2 | 2.2 | 149 |
| 14640_at | At2g39200 | putative Mlo protein | defense | 1.0 | 3.2 | 5.3 | 3.9 | 1.0 | 1.0 | 57 |
| 16440_at | At2g40000 | putative nematode-resistance protein | defense | 3.6 | 4.2 | 2.9 | −1.3 | −1.2 | −3.7 | 297 |
| 20384_at | At4g36010 | thaumatin-like protein | defense | 1.0 | 1.6 | 11.3 | 21.4 | 11.8 | 2.4 | 274 |
| 17932_at | At1g05250 | putative peroxidase (ATP12a) | detoxification | 1.0 | 6.0 | 5.5 | 2.9 | −2.0 | 6.7 | 167 |
| 20060_at | At1g10370 | GST30/ERD9 | detoxification | 1.0 | 1.2 | 2.1 | 5.8 | 3.6 | 1.4 | 528 |
| 20438_at | At1g10370 | GST30/ERD9 | detoxification | 1.0 | 1.0 | 2.2 | 5.7 | 3.1 | 1.0 | 238 |
| 18963_at | At2g29440 | putative glutathione S-transferase | detoxification | 1.0 | 2.4 | 5.1 | 10.2 | 1.0 | 1.3 | 120 |
| 18150_at | At2g39040 | putative peroxidase | detoxification | 1.6 | 7.7 | 5.8 | 3.1 | 1.0 | 2.3 | 67 |
| 16971_s_at | At3g01190 | peroxidase (prxr7) | detoxification | 1.0 | 7.5 | 6.4 | 7.8 | 1.0 | 3.7 | 77 |
| 19592_at | At3g49960 | peroxidase (ATP21a) | detoxification | 1.0 | 5.5 | 4.3 | 1.9 | 1.0 | 2.5 | 55 |
| 16028_at | At4g30170 | peroxidase (ATP8a) | detoxification | 1.0 | 5.6 | 5.6 | 4.1 | −1.8 | 5.3 | 2212 |
| 20275_at | At4g37320 | cytochrome P450-like protein | detoxification | 1.0 | 1.0 | 1.0 | 1.0 | 3.3 | 1.0 | 163 |
| 17942_s_at | At5g17820 | peroxidase (prxr10) | detoxification | 1.0 | 4.9 | 4.2 | 2.2 | −2.3 | 3.2 | 156 |
| 19622_g_at | At5g42180 | peroxidase (prxr4) | detoxification | 1.5 | 7.0 | 6.4 | 5.5 | 1.0 | 3.8 | 45 |
| 20296_s_at | At5g67400 | peroxidase (prxr11) | detoxification | 1.0 | 6.9 | 4.4 | 3.6 | 1.0 | 6.0 | 102 |
| 16621_s_at | At1g12370 | CPD photolyase (PHR1) | DNA repair | 1.0 | 1.1 | 1.2 | 3.5 | 4.9 | 1.8 | 85 |
| 16896_s_at | At2g41260 | LEA M17 protein | LEA/dehydrin | 1.0 | 2.7 | 5.7 | 2.6 | 1.4 | 1.0 | 34 |
| 19152_at | At5g06760 | LEA D113 protein | LEA/dehydrin | 1.0 | 1.0 | 1.4 | 13.7 | 9.6 | 1.3 | 135 |
| 17407_s_at | At5g52300 | RD29B | LEA/dehydrin | 1.0 | 1.0 | 1.3 | 2.5 | 6.2 | 1.3 | 57 |
| Unknown role | | | | | | | | | | |
| 13474_at | At2g40900 | putative integral membrane protein | unknown | 1.0 | 1.8 | 2.6 | 5.9 | 1.6 | 1.0 | 218 |
| 20686_at | At3g16860 | hypothetical protein (SEB2) | unknown | 1.0 | 2.2 | 5.0 | 2.5 | 1.0 | 1.0 | 198 |
| 19190_g_at | At3g21490 | farnesylated protein (ATFP6) | unknown | 1.0 | 1.0 | 1.8 | 4.6 | 6.6 | 2.8 | 3131 |
| 14105_at | At4g12750 | putative cytochrome c family protein | unknown | 1.2 | 1.0 | 1.2 | 1.6 | 4.1 | 1.6 | 119 |
| 19976_at | At4g38400 | putative pollen allergen | unknown | 1.0 | 1.4 | 7.3 | 13.7 | 5.5 | 1.0 | 231 |
| 19178_at | At5g20230 | blue copper binding protein (Atbcd) | unknown | 2.1 | 2.5 | 4.5 | 4.3 | 4.9 | 1.3 | 1588 |
| Unknown | | | | | | | | | | |
| proteins | | | | | | | | | | |
| 17675_at | At1g03520 | unknown protein | unknown | 1.0 | 1.2 | 2.0 | 5.0 | 8.4 | 2.7 | 99 |
| 18383_at | At1g04570 | putative protein | unknown | 1.0 | 1.0 | 7.9 | 35.1 | 11.0 | 3.8 | 361 |
| 16688_at | At1g10080 | hypothetical protein | unknown | ND | 1.6 | 2.3 | 7.8 | 8.2 | 1.9 | 145 |
| 12132_at | At1g10270 | unknown protein | unknown | 1.0 | 1.0 | 1.0 | 4.1 | 7.1 | 1.0 | 83 |
| 12187_at | At1g10410 | unknown protein | unknown | 1.0 | 1.0 | 2.1 | 6.2 | 7.5 | 3.1 | 271 |
| 15921_s_at | At1g10410 | unknown protein | unknown | 1.0 | 1.0 | 1.6 | 5.5 | 6.0 | 2.2 | 337 |
| 18995_at | At1g11960 | hypothetical protein | unknown | 1.6 | 1.7 | 5.4 | 6.7 | 1.0 | 1.5 | 66 |
| 20367_s_at | At1g11960 | unknown protein | unknown | 1.0 | 5.2 | 5.8 | 3.8 | 1.0 | 4.3 | 48 |
| 15931_at | At1g21670 | hypothetical protein | unknown | 1.0 | 1.0 | 1.6 | 2.1 | 4.3 | 2.7 | 432 |
| 13920_at | At1g23710 | unknown protein | unknown | 3.9 | 7.2 | 3.3 | 1.6 | 1.3 | 1.0 | 169 |
| 17179_at | At1g49450 | hypothetical protein | unknown | 1.0 | 1.0 | 2.5 | 4.0 | 6.0 | 2.8 | 46 |
| 12018_at | At1g64890 | hypothetical protein | unknown | 1.0 | 1.0 | 3.1 | 6.4 | 12.7 | 3.3 | 120 |
| 14019_at | At2g17280 | unknown protein | unknown | 1.0 | 1.3 | 1.0 | 3.9 | 5.2 | 1.0 | 62 |
| 14412_at | At2g18900 | unknown protein | unknown | 1.0 | 1.0 | 1.0 | 1.6 | 3.5 | 1.0 | 92 |
| 15389_at | At2g22860 | unknown protein | unknown | 1.0 | 1.7 | 2.5 | 2.9 | 4.8 | 1.0 | 318 |
| 16995_at | At2g23170 | unknown protein | unknown | 1.0 | 1.0 | 1.0 | 1.0 | 5.7 | 1.0 | 55 |
| 15392_at | At2g27080 | unknown protein | unknown | 1.8 | 4.7 | 9.5 | 2.8 | 1.6 | 1.0 | 111 |
| 14924_at | At2g28400 | hypothetical protein | unknown | 1.2 | 1.0 | 3.1 | 8.0 | 4.6 | 2.5 | 267 |
| 18267_at | At2g32210 | unknown protein | unknown | 1.7 | 3.6 | 4.4 | 2.3 | 4.5 | 1.8 | 156 |
| 18885_at | At2g36220 | unknown protein | unknown | 1.8 | 3.1 | 4.3 | 3.4 | 3.4 | 1.4 | 151 |
| 12128_at | At2g41010 | unknown protein | unknown | 3.2 | 9.8 | 4.3 | 1.4 | 1.0 | 1.0 | 353 |
| 13426_at | At2g41190 | unknown protein | unknown | 1.0 | 1.0 | 1.0 | 1.3 | 3.8 | 1.0 | 47 |
| 18631_at | At2g41640 | unknown protein | unknown | 3.1 | 8.3 | 6.7 | 1.0 | 1.0 | 1.0 | 96 |
| 17231_at | At2g46970 | hypothetical protein | unknown | 1.0 | 1.0 | 1.0 | 6.5 | 4.0 | 1.0 | 57 |
| 20005_s_at | At2g47990 | unknown protein | unknown | 1.0 | 1.2 | 2.1 | 3.6 | 4.7 | 1.8 | 102 |
| 15422_at | At4g04330 | hypothetical protein | unknown | 1.0 | 1.0 | 2.0 | 2.4 | 5.0 | 1.8 | 1017 |
| 18778_at | At4g15430 | hypothetical protein | unknown | 1.0 | 1.0 | 2.4 | 4.4 | 2.7 | 1.5 | 161 |
| 12561_at | At4g19120 | putative protein | unknown | 1.0 | 1.0 | 1.2 | 3.0 | 3.6 | 1.4 | 253 |
| 12027_at | At4g20170 | putative protein | unknown | 1.0 | 1.0 | 2.3 | 5.4 | 1.2 | 1.0 | 161 |
| 14398_at | At4g21570 | putative protein | unknown | 1.0 | 1.0 | 1.4 | 3.1 | 4.3 | 1.9 | 424 |
| 15319_at | At4g23500 | putative protein | unknown | 1.0 | 1.2 | 1.4 | 1.7 | 4.2 | 1.8 | 78 |
| 14835_at | At4g25730 | putative protein | unknown | 1.0 | 1.0 | 1.0 | 1.3 | 4.4 | 1.7 | 44 |
| 15431_at | At4g27280 | putative protein | unknown | 9.2 | 10.3 | 1.5 | ND | 1.5 | −1.3 | 1052 |
| 13352_at | At4g27360 | putative protein | unknown | 2.0 | 3.0 | 12.6 | 29.6 | 1.4 | 1.0 | 345 |
| 17860_at | At4g27410 | putative protein | unknown | 1.1 | 1.4 | 2.2 | 4.9 | 4.3 | 2.1 | 401 |
| 12117_at | At4g27520 | putative protein | unknown | 1.2 | 1.0 | 1.5 | 2.5 | 3.7 | 1.8 | 2379 |
| 15083_at | At4g32190 | putative protein | unknown | 1.0 | 1.0 | 3.0 | 9.0 | 5.0 | 1.0 | 954 |
| 16753_at | At4g33920 | putative protein | unknown | 2.2 | 5.1 | 8.4 | 4.9 | 4.8 | 1.8 | 110 |
| 15084_at | At4g35320 | putative protein | unknown | 1.0 | 1.0 | 3.2 | 4.4 | 1.6 | 1.0 | 160 |
| Mean fold-change values at each time-point are shown with fold-change values associated with a difference call of “No Change” converted to 1.0. Fold-change values, where the data passed the criteria for 3-fold up-regulation, are in boldface. ND indicates where the four difference calls associated with a time-point included both “Increase” and “Decrease” calls | | | | | | | | | | |
| #and therefore a mean fold-change was not determined. Peak AD is the greatest of the mean average difference values obtained during the cold treatment. | | | | | | | | | | |

TABLE 3
Genes down-regulated by cold.
Genes down-regulated by cold

| | AGI | | | Time (h) | | | | | | |
|---|---|---|---|---|---|---|---|---|---|---|
| Probe set | identifier | Description | Sub-role | 0.5 | 1 | 4 | 8 | 24 | 168 | T/L |
| Metabolism | | | | | | | | | | |
| 15591_s_at | At5g18170 | glutamate dehydrogenase 1 (GDH1) | amino acid | 1.6 | 1.0 | 1.0 | 1.0 | 5.3 | 1.0 | T |
| 16593_at | At1g28670 | lipase | lipid | 1.0 | 1.0 | 1.7 | 3.8 | 4.2 | 2.4 | T |
| 17476_at | At2g35690 | putative acyl-CoA oxidase | lipid | 1.1 | 1.0 | 1.9 | 1.3 | 4.0 | 1.6 | T |
| 15646_s_at | At1g55920 | serine acetyltransferase (SAT1) | nitrogen and sulfur | 1.0 | 1.0 | 3.0 | 4.6 | 2.1 | 1.0 | T |
| 12790_s_at | At4g22690 | cytochrome P450 like protein | secondary | 1.0 | 1.0 | 1.1 | 2.3 | 6.1 | 3.4 | L |
| 18977_at | At1g10360 | GST29 | secondary | 1.0 | 1.0 | 1.5 | 2.3 | 3.7 | 1.8 | T |
| 20442_i_at | At1g16410 | putative cytochrome P450 | secondary | 1.0 | 1.0 | 1.2 | 1.4 | 4.3 | 1.4 | T |
| 19549_s_at | At2g22330 | putative cytochrome P450 | secondary | 1.0 | 1.7 | 1.2 | 1.0 | 3.4 | 1.0 | T |
| 17957_at | At2g23600 | putative acetone-cyanohydrin lyase | secondary | 1.0 | 1.0 | 1.2 | 1.4 | 2.2 | 4.4 | L |
| 18581_at | At2g29340 | putative tropinone reductase | secondary | 1.0 | 1.0 | 1.0 | 1.4 | 4.5 | 2.1 | T |
| 16603_s_at | At4g15550 | UDP-glucose: indole-3-acetate beta-D- | secondary | 1.0 | 1.0 | 1.3 | 1.4 | 5.1 | 3.5 | L |
| | | glucosyltransferase | | | | | | | | |
| 19704_i_at | At5g24160 | squalene epoxidase homologue (Sqp1;2) | secondary | 1.0 | 1.0 | 1.0 | 1.0 | 5.6 | 5.9 | L |
| 15144_s_at | At5g14740 | carbonic anhydrase (Ca180) | carbohydrate | 1.0 | 1.0 | ND | 1.1 | 2.7 | 5.8 | L |
| 16428_at | At3g01500 | carbonic anhydrase, chloroplast precursor | carbohydrate | 1.2 | 1.0 | 1.0 | 1.2 | 1.8 | 9.2 | L |
| Energy | | | | | | | | | | |
| 16983_at | At1g10960 | ferredoxin precusor isolog | photosynthesis | 1.0 | 1.0 | 1.0 | 1.0 | 2.2 | 8.6 | L |
| 13678_at | At1g19150 | PSI type II chlorophyll a/b-binding protein | photosynthesis | 1.0 | 1.0 | 1.0 | 1.4 | 3.2 | 4.4 | L |
| | | (Lhca2*1) | | | | | | | | |
| 16899_at | At1g51400 | Putative photosystem II 5 KD protein; | photosynthesis | 1.0 | 1.0 | 1.5 | 3.2 | 11.1 | 7.1 | L |
| 18665_r_at | At2g20260 | photosystem I subunit IV precursor (psaE2) | photosynthesis | ND | ND | ND | 2.6 | 5.7 | 2.0 | T |
| 17054_s_at | At2g40100 | Lhcb4: 3 protein | photosynthesis | 1.0 | 1.4 | 1.2 | 1.7 | 1.0 | 15.1 | L |
| Transcription | | | | | | | | | | |
| 18590_at | At1g69490 | putative NAC family transcription factor | mRNA synthesis | 1.0 | 1.0 | 5.3 | 6.3 | 1.0 | 1.0 | T |
| | | (NAP) | | | | | | | | |
| 20499_at | At2g33480 | putative NAM (no apical meristem)-like | mRNA synthesis | 1.0 | 1.0 | 4.3 | 6.4 | 4.7 | 1.3 | T |
| | | protein | | | | | | | | |
| 19232_at | At4g14540 | CCAAT-binding transcription factor | mRNA synthesis | 1.0 | 1.0 | 2.0 | 3.9 | 1.5 | 1.8 | T |
| | | subunit (AtNF-YB-3) | | | | | | | | |
| 12522_at | At4g39780 | AP2 domain containing protein (RAP2.4) | mRNA synthesis | 1.4 | 1.0 | 3.4 | 4.4 | 4.3 | 2.7 | T |
| 16978_g_at | At4g40060 | homeodomain leucine-zipper protein | mRNA synthesis | 1.0 | 1.0 | 2.0 | 1.6 | 3.7 | 5.8 | T |
| | | (ATHB16) | | | | | | | | |
| 12727_f_at | At5g07700 | putative transcription factor (MYB76) | mRNA synthesis | ND | 1.0 | 1.7 | 1.0 | 5.6 | 1.7 | T |
| 20354_s_at | At1g09140 | putative SF2 ASF splicing modulator | mRNA processing | 1.0 | 1.0 | 2.2 | 2.3 | 2.8 | 4.3 | L |
| 16069_s_at | At1g23860 | 9G8-like splicing factor (SRZ21) | mRNA processing | 1.0 | 1.0 | 1.1 | 1.4 | 3.4 | 5.0 | L |
| Protein | | | | | | | | | | |
| fate | | | | | | | | | | |
| 12858_at | At4g26160 | thioredoxin-like 2 | protein modification | 2.1 | 1.0 | 1.7 | 3.7 | 1.7 | 1.0 | T |
| 20256_s_at | At2g22990 | putative serine carboxypeptidase I | proteolysis | 1.0 | 1.1 | 1.0 | 1.0 | 2.4 | 4.5 | L |
| 19892_at | At2g38870 | putative protease inhibitor | proteolysis | 1.0 | 1.0 | 1.0 | 1.5 | 3.7 | 5.1 | L |
| Transport | | | | | | | | | | |
| facilitation | | | | | | | | | | |
| 12769_at | At2g37170 | plasma membrane intrinsic protein (PIP2;2) | other | 1.0 | 1.7 | 1.7 | 1.5 | 4.0 | 2.2 | T |
| 16005_at | At4g17340 | tonoplast intrinsic protein (TIP2;2) | other | 1.0 | 2.5 | 2.8 | 2.2 | 2.5 | 7.3 | L |
| 17572_s_at | At1g64780 | ammonium transporter (ATM1) | ion transporters | 1.0 | 1.0 | 1.2 | 2.5 | 1.7 | 4.3 | L |
| Cellular | | | | | | | | | | |
| biogenesis | | | | | | | | | | |
| 19017_at | At4g37800 | endo-xyloglucan transferase-like protein | cell wall | 1.0 | 1.0 | 1.8 | 1.6 | 2.8 | 3.9 | L |
| 12523_at | At1g69530 | expansin (At-EXP1) | cell wall | 1.0 | 1.0 | 3.1 | 2.8 | 3.1 | 3.8 | L |
| 12515_at | At2g39700 | putative expansin | cell wall | 1.0 | 1.0 | 1.2 | 2.1 | 4.4 | 2.7 | L |
| 19660_at | At2g40610 | putative expansin | cell wall | 1.0 | 1.2 | 5.1 | 5.7 | 3.8 | 1.7 | T |
| 16457_s_at | At2g46330 | arabinogalactan protein (AGP16) | cell wall | 1.0 | 1.0 | 1.2 | 3.4 | 2.2 | 1.3 | T |
| 19118_s_at | At4g12550 | putative cell wall-plasma membrane | cell wall | ND | 4.0 | 3.5 | 4.2 | 6.9 | 8.2 | T |
| | | disconnecting protein (AIR1A) | | | | | | | | |
| 19905_at | At4g19420 | putative pectinacetylesterase | cell wall | 1.5 | 1.0 | 1.9 | 4.4 | 7.3 | 6.8 | L |
| 12312_at | At4g24780 | putative pectate lyase | cell wall | 1.0 | 1.0 | 1.3 | 1.2 | 4.2 | 2.0 | T |
| 12313_g_at | At4g24780 | putative pectate lyase | cell wall | 1.0 | 1.0 | 1.1 | 1.0 | 3.9 | 2.3 | T |
| Cellular | | | | | | | | | | |
| communication | | | | | | | | | | |
| and signal | | | | | | | | | | |
| transduction | | | | | | | | | | |
| 13614_at | At1g04350 | putative 1-aminocyclopropane-1- | intercellular | 1.0 | 1.0 | 1.2 | 1.4 | 3.6 | 4.9 | L |
| | | carboxylate oxidase | communication | | | | | | | |
| 17323_at | At1g11050 | Ser/Thr protein kinase isolog | intracellular signaling | 1.2 | 1.8 | 1.3 | 3.0 | 4.4 | 1.9 | T |
| 16140_s_at | At1g21270 | wall-associated kinase 2 (WAK2) | intracellular signaling | 2.0 | 2.9 | 3.2 | 5.4 | 1.2 | 1.6 | T |
| 13680_at | At1g55020 | lipoxygenase 1 (LOX1) | intercellular | 1.0 | 1.5 | 1.9 | 1.2 | 10.0 | 10.2 | L |
| | | | communication | | | | | | | |
| 18253_s_at | At1g76680 | 12-oxophytodienoate reductase (OPR1) | intercellular | 1.0 | 1.3 | 1.0 | 1.0 | 3.9 | 2.7 | T |
| | | | communication | | | | | | | |
| 18202_at | At2g15680 | calmodulin-like protein | intracellular signaling | 1.0 | 1.0 | 1.4 | 2.5 | 3.6 | 3.8 | L |
| 16818_at | At4g21410 | putative serine/threonine kinase | intracellular signalling | 1.0 | 1.0 | 1.0 | 1.3 | 2.6 | 5.0 | L |
| 12968_at | At4g28270 | RING zinc finger protein (A-RZF) | intracellular signalling | 1.0 | 1.0 | 4.9 | 5.5 | 6.9 | 3.1 | T |
| Cell rescue, | | | | | | | | | | |
| defence, cell | | | | | | | | | | |
| death and aging | | | | | | | | | | |
| 15098_s_at | At4g35770 | senescence-associated protein (sen1) | aging | 1.0 | 1.0 | 1.0 | 2.7 | 7.3 | 5.7 | L |
| 14636_s_at | At1g75040 | thaumatin-like protein | defense | 2.6 | 4.8 | 3.3 | 2.1 | 2.0 | 2.7 | T |
| 17840_at | At2g43570 | putative endochitinase | defense | 1.0 | 4.7 | 4.4 | 3.6 | 1.0 | 4.5 | L |
| 15965_at | At3g16460 | myrosinase binding protein-like | defense | 1.0 | 1.8 | 2.3 | 1.0 | 3.9 | 2.2 | T |
| 13212_s_at | At3g57260 | beta-1, 3-glucanase (BG2) | defense | 2.5 | 14.1 | 22.8 | 3.6 | 1.3 | 2.7 | T |
| 19034_at | At4g19530 | TMV resistance protein N-like | defense | 1.0 | 1.3 | 1.0 | 1.5 | 3.8 | 3.1 | T |
| 18966_at | At2g29420 | putative glutathione S-transferase (GST25) | detoxification | 1.0 | 1.2 | 1.0 | 1.0 | 5.5 | 6.5 | L |
| 15982_s_at | At2g37130 | putative peroxidase (ATP2a) | detoxification | 1.0 | 1.0 | 1.6 | 1.2 | 4.1 | 2.0 | T |
| 17930_s_at | At4g37520 | peroxidase (prxr2) | detoxification | 1.1 | 1.0 | 1.3 | 2.6 | 14.6 | 2.8 | T |
| 15985_at | At5g64100 | peroxidase (ATP3a) | detoxification | 1.0 | 2.5 | 2.2 | 2.4 | 4.3 | 2.6 | T |
| 14067_at | At2g19310 | putative small heat shock protein | stress | 1.2 | 1.0 | 3.0 | 5.8 | 6.2 | 6.5 | L |
| 13284_at | At3g12580 | heat shock protein (HSP70) | stress | 1.0 | 1.0 | 1.3 | 1.4 | 1.0 | 9.4 | L |
| 19227_at | At4g13830 | DnaJ-like protein | stress | 1.0 | 1.0 | 1.6 | 3.2 | 1.7 | 2.5 | T |
| 13285_at | At5g52640 | heat shock protein (HSP83) | stress | 1.6 | 1.2 | 1.7 | 1.5 | 5.3 | 6.5 | L |
| Unknown role | | | | | | | | | | |
| 20190_at | At2g16660 | nodulin-like protein | unknown | 1.0 | 1.4 | 1.7 | 2.2 | 2.2 | 4.1 | L |
| 19565_at | At2g30810 | GASA5 | unknown | 1.0 | 1.0 | 1.6 | 2.2 | 5.2 | 3.7 | L |
| 16046_s_at | At3g26740 | putative light regulated protein | unknown | 1.4 | 1.0 | 1.3 | 3.1 | 1.9 | 1.9 | T |
| 19695_at | At4g38840 | auxin-induced protein-like | unknown | 1.0 | 1.0 | 4.2 | 1.7 | 2.9 | 2.8 | T |
| Unknown | | | | | | | | | | |
| proteins | | | | | | | | | | |
| 15045_at | At1g23205 | unknown protein | unknown | 1.0 | 1.0 | 1.0 | 1.0 | 3.8 | 4.5 | L |
| 17909_at | At1g62480 | unknown protein | unknown | 1.0 | 1.0 | 1.0 | 1.1 | 2.6 | 3.6 | L |
| 19402_at | At2g04690 | unknown protein | unknown | 1.0 | 1.2 | 2.3 | 3.6 | 2.6 | 2.0 | T |
| 14704_s_at | At2g14560 | unknown protein | unknown | 3.8 | 7.3 | 14.0 | 7.5 | 1.5 | 1.9 | T |
| 12843_s_at | At2g16590 | unknown protein | unknown | 2.0 | 2.2 | 3.2 | 6.0 | 5.8 | 4.0 | L |
| 12765_at | At2g22660 | unknown protein | unknown | 2.0 | 1.0 | 1.0 | 1.0 | 3.3 | 1.0 | T |
| 18416_at | At2g24150 | hypothetical protein | unknown | 4.2 | 1.0 | 4.2 | 14.8 | 17.8 | 5.0 | T |
| 19901_at | At2g32880 | unknown protein | unknown | 1.3 | 1.8 | 1.3 | 1.0 | 1.0 | 3.7 | L |
| 13586_g_at | At2g34170 | unknown protein | unknown | 1.0 | 1.0 | 1.0 | 1.0 | 4.6 | 2.5 | T |
| 19387_at | At2g35820 | unknown protein | unknown | 1.0 | 1.0 | 1.0 | 2.3 | 3.9 | 3.2 | L |
| 12799_at | At2g37340 | unknown protein | unknown | 1.0 | 1.0 | 1.0 | 1.0 | 1.0 | 3.8 | L |
| 19363_at | At2g42610 | unknown protein | unknown | 1.0 | 1.7 | 2.2 | 1.7 | 1.2 | 3.9 | L |
| 15552_at | At3g46780 | putative protein | unknown | 1.1 | 1.0 | 1.2 | 1.0 | 1.6 | 3.7 | L |
| 12212_at | At3g52070 | putative protein | unknown | 1.2 | 1.2 | 2.5 | 3.9 | 1.2 | 1.0 | T |
| 18626_at | At4g00780 | unknown protein | unknown | 1.0 | 1.0 | 1.2 | 1.4 | 3.6 | 16.8 | L |
| 20429_at | At4g14400 | hypothetical protein | unknown | 4.5 | 4.6 | 8.9 | 8.5 | 6.6 | 1.0 | T |
| 12815_at | At4g27450 | putative protein | unknown | 1.0 | 1.0 | 2.0 | 4.9 | 6.3 | 4.5 | T |
| 12169_i_at | At4g33960 | putative protein | unknown | 1.0 | 1.0 | 2.4 | 5.7 | 2.5 | 2.2 | T |
| 13146_s_at | At4g35750 | putative protein | unknown | 1.0 | 1.0 | 2.4 | 3.8 | 2.9 | 1.0 | T |
| Non-coding | | | | | | | | | | |
| sequences | | | | | | | | | | |
| 18060_i_at | ˜ | snoRNA (U25b) | | 1.0 | 1.0 | 2.8 | 3.6 | 3.3 | 1.7 | T |
| 12846_s_at | ˜ | Intergenic region of 25S-18S ribosomal | | ND | 2.2 | 2.2 | 3.3 | 2.2 | 8.3 | L |
| | | DNA spacer | | | | | | | | |
| Probe sets designated as long-term (L) had fold-change values of −3 or less associated with difference calls of “Decrease” in all four comparisons between the 7 day time-point and 0 hour. All other cold down-regulated genes were designated transient (T). Mean fold-change values at each time-point are shown with fold- change values associated with a difference call of | | | | | | | | | | |
| #“No Change” converted to −1.0. Fold-change values where the data passed the criteria for 3-fold down-regulation (see M boldface. ND indicates where the four difference calls associated with a time-point included both “Increase” and “Decrease” calls and therefore a mean fold-change was not determined. | | | | | | | | | | |

The CBF cold-response pathway is one low temperature “gene network” that contributes to cold tolerance in Arabidopsis. A fundamental question addressed by the present invention is whether other low temperature gene networks contribute significantly to freezing tolerance or other aspects of growth and development at low temperature. To address this issue, arrays known in the art under the tradename AFFYMETRIX GENECHIP were used to analyze the Arabidopsis transcriptome at multiple times after transferring plants from warm to cold temperature and in warm-grown plants that constitutively express either CBF1, 2 or 3. The results indicate that a dynamic series of changes in the Arabidopsis transcriptome is set in motion upon transferring plants from warm to cold temperatures that includes cold regulatory gene networks in addition to the CBF cold-response pathway. In addition, the results identify more than 250 newly described cold-responsive genes that offer explanations for certain biochemical changes that occur during cold acclimation, identify candidate polypeptides with roles in cold tolerance, and indicate that gene repression may have an integral role in the cold acclimation response.

AFFYMETRIX GENECHIP arrays, which contain 8297 DNA oligonucleotide probe sets representing approximately 8000 genes per chip, were used to assay changes in the Arabidopsis transcriptome in response to exposing plants to low temperature. Transcript levels were analyzed in duplicate biological samples harvested just before plants were transferred from 22° C. to 4° C. (“warm” sample) and then at 0.5 h, 1 h, 4 h, 8 h, 24 h and 7 d after transfer (where h=hour and d=day). Fold-change values were calculated for the duplicate samples harvested at 4° C. compared to each of the two warm samples, thus generating four comparisons for each time-point. A gene was designated as being up-regulated at a given time-point if the signal intensity was above background (“present”) for both duplicate cold samples, if there was a difference call of “increase” for all four comparisons, and if the fold-increase value was greater than or equal to 3 for all four comparisons. Similarly, a gene was designated as being down-regulated at a given time-point if the transcript levels produced a hybridization intensity above background for both duplicate warm samples, if there was a difference call of “decrease” for all four comparisons, and if the fold-decrease value was greater than or equal to 3 for all four comparisons. Control experiments indicated that using a cutoff of three-fold would effectively rule out the possibility of a gene being inappropriately designated as “cold-responsive” due to a technical error. When a single RNA sample from warm-grown plants was used to prepare two probes that were hybridized to two different GENECHIPS, only three out of the 8297 total probe sets were found to have a difference call of “increase” or “decrease” and a fold-change of three or greater, corresponding to a false positive rate of 0.04 percent. Thus, in using four comparisons to select cold-responsive genes, the false positive rate would be predicted to be less than 2 per 1014 genes.

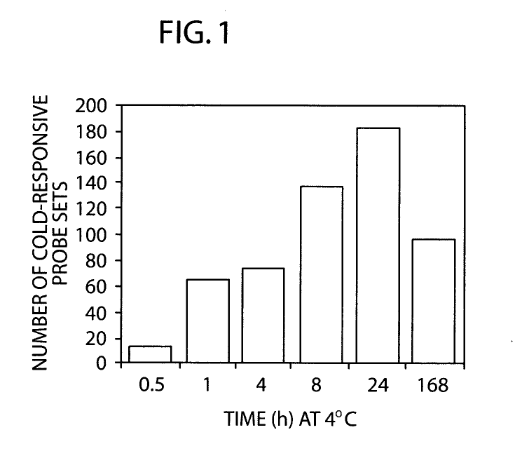

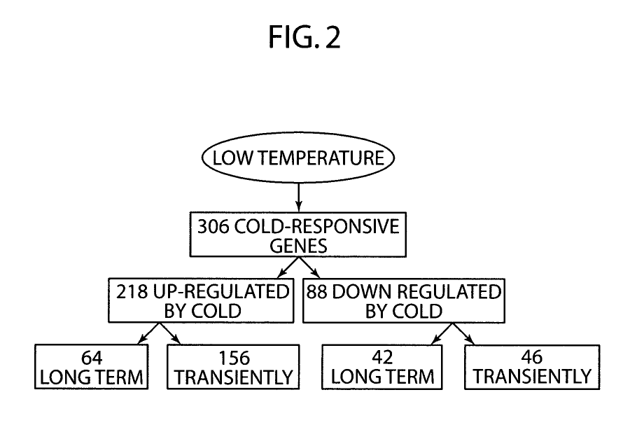

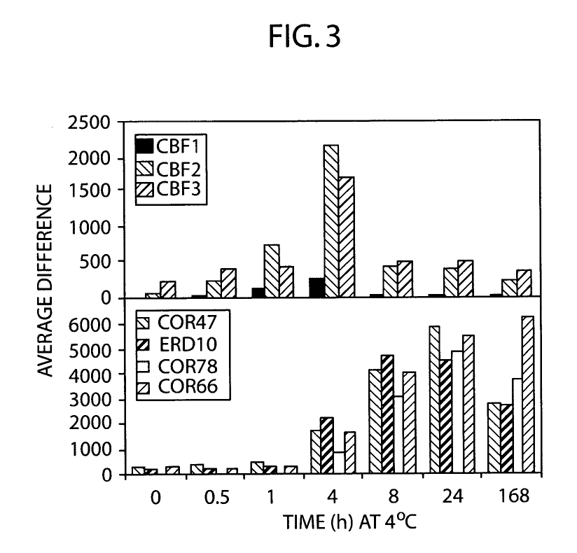

Using the described criteria, it was found that a total of 330 probe sets represented cold-responsive genes, corresponding to 4 percent of the total probe sets. The number of cold-responsive probe sets increased to a maximum of 182 at 24 h and then declined to 97 at 7 d (FIG. 1). Analysis of these data indicated that 306 genes (some genes are represented by more than one probe set), were cold-responsive at a minimum of one time-point during the course of the experiment (FIG. 2 and Tables 1, 2 and 3). Of these, 218 genes were scored as being up-regulated in response to low temperature and another 88 as down-regulated (FIG. 2). As expected, among the cold-regulated genes were members of the CBF cold-response pathway. In particular, the transcript levels for CBF1, 2 and 3 increased within the first hour of plants being exposed to low temperature followed closely (within 4 h) by expression of known CBF-target genes including COR6.6, ERD10, COR47 and COR78 (FIG. 3). These results indicated the tissue samples used in this experiment were undergoing a typical cold acclimation response and that the GENECHIPS replicated results previously obtained by Northern Blot analysis (Gilmour et al., 1998; Liu et al., 1998; Shinwari et al., 1998).

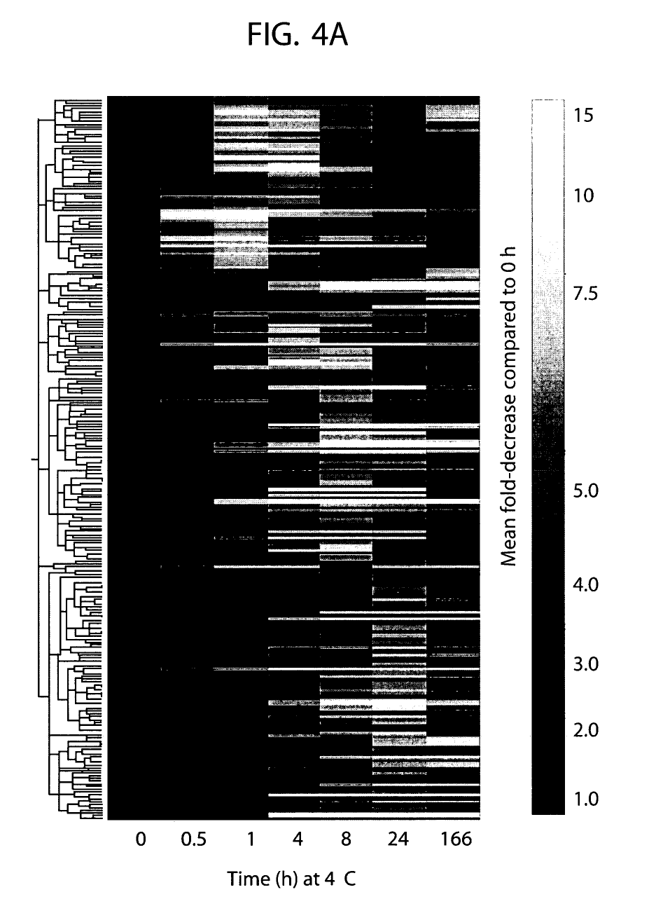

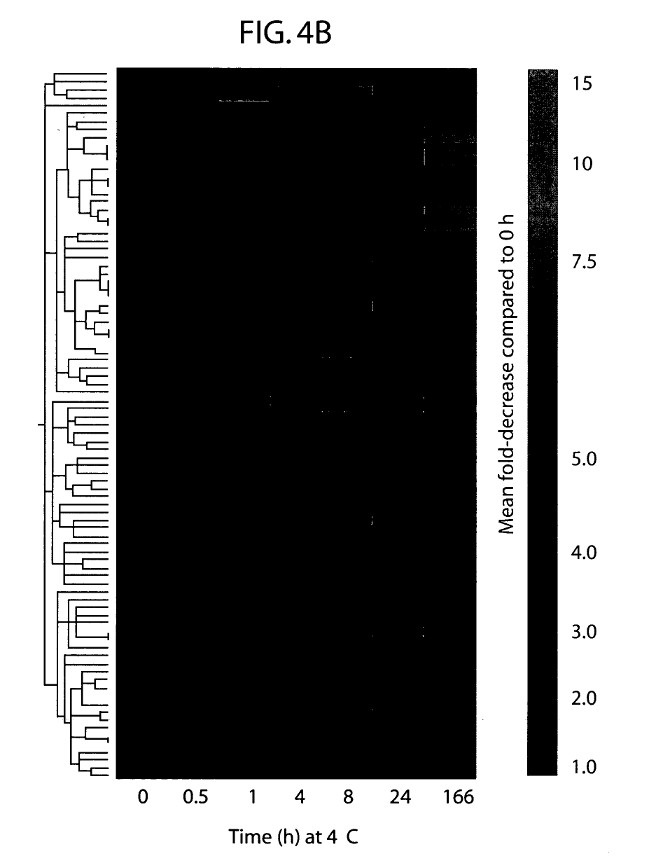

Hierarchical clustering of the entire set of 218 up-regulated genes revealed that blocks of genes were induced in multiple waves after transferring plants to low temperature (FIG. 4A). Additionally, it was evident that some genes were transiently expressed, while others were induced and remained activated for the entire seven-day experiment. Overall, the kinetic pattern observed did not fit a “simple” two-step cascade profile of the CBF cold-response pathway-rapid cold induction of the CBF transcriptional activators (during the first hour) followed by expression of the CBF regulon (by 4 hours)—suggesting that multiple regulatory pathways were activated in response to the temperature downshift. A similar picture emerged with the set of 88 down-regulated genes (FIG. 4B).

Transferring plants from warm to cold temperature triggers the cold acclimation response that includes expression of COR and other genes that remain up-regulated for extended periods of time at low temperature. Of the 218 genes determined to be up-regulated in response to cold, (i.e., were up-regulated at least 3-fold at one or more time-points during the course of the experiment), 64 genes remained up-regulated at least 2.5-fold at 7 d. These were considered “long-term” up-regulated genes (FIGS. 5A and. 6 and Table 1). The lower threshold of 2.5-fold was used to designate a gene as being long-term up-regulated by cold since the expression levels of genes that reach a peak of 3-fold or greater may have declined below this level by 7 days, but may not have returned to pre-stress levels. From a search of the literature, it appeared that 50 of these long-term up-regulated genes had not been previously reported to be cold-regulated in Arabidopsis (Table 1).

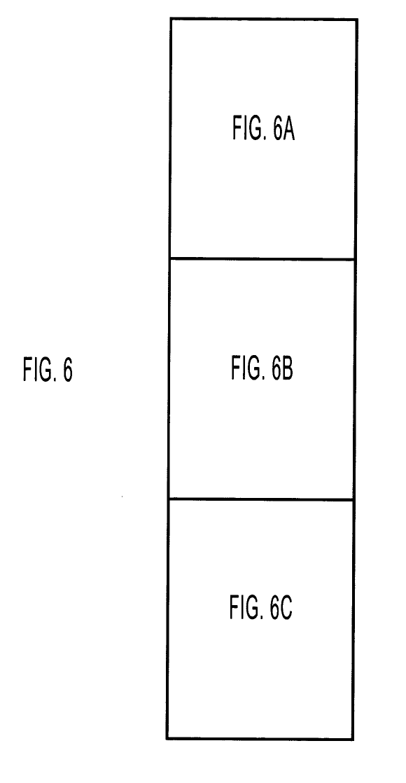

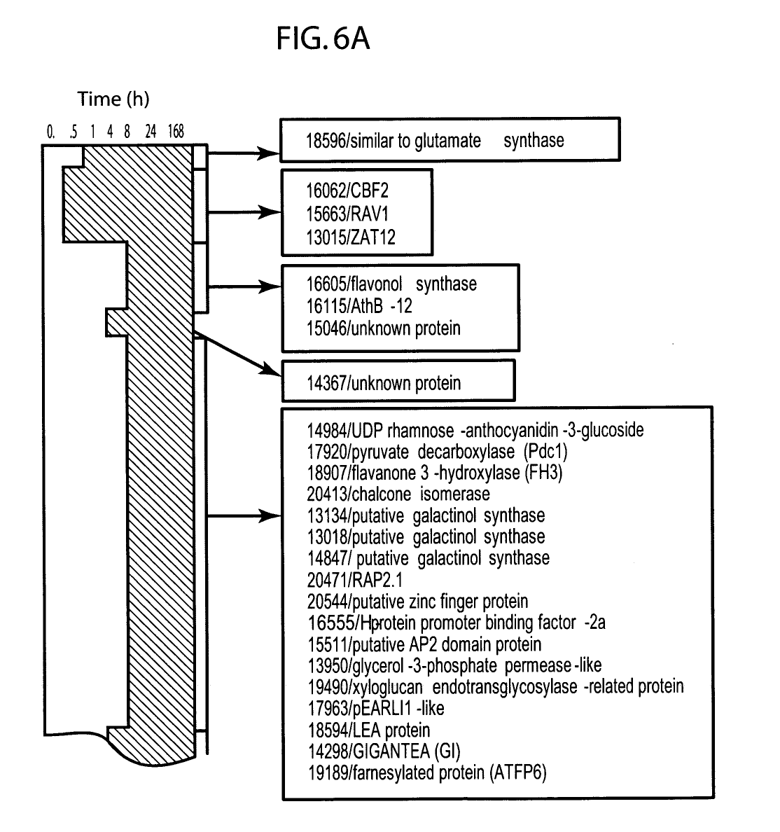

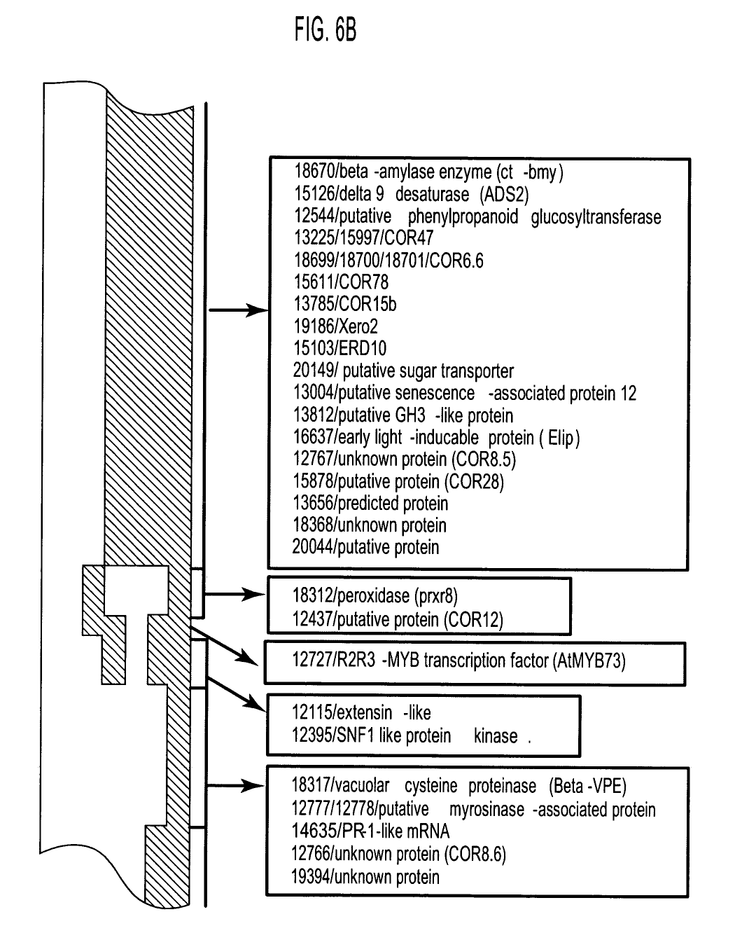

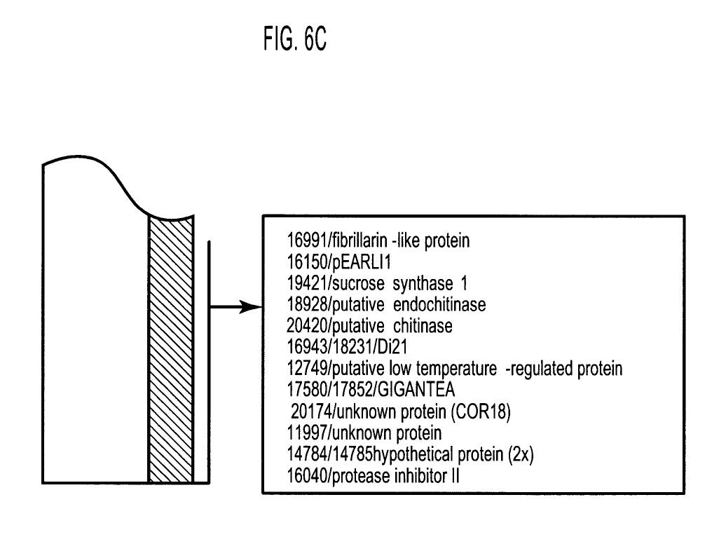

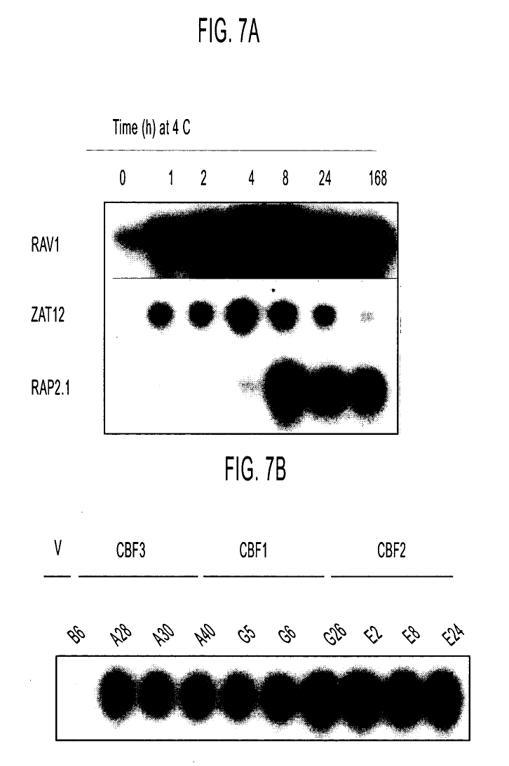

Hierarchical clustering indicated that the 64 long-term up-regulated genes were “induced” at different times following transfer of plants to low temperature (FIGS. 5A and 6). This was most easily visualized using a “binary” hierarchical clustering format (FIG. 6). In this case, genes that were up-regulated 2.5-fold or more were considered to be induced and time-points where this occurred were indicated as colored hatched, while those that were not changed by 2.5-fold were considered “unchanged” and these time-points were indicated as white (all zero time values were thus white). The presentation reveals that blocks of genes were induced at each time-point after transferring plants to low temperature suggesting that multiple regulatory pathways were likely to be involved in their induction. Consistent with this notion was the finding that in addition to CBF2, eight other long-term cold-responsive genes that encoded either known or putative transcription factors were induced at various times during the course of the experiment (FIG. 6 and Table 1). Two of these genes were induced rapidly in response to low temperature in parallel with CBF2, namely (and for illustrative purposes of cold-regulated transcription factors) RAV1 (At1g13260, Kagaya et al., Nucleic Acids Res. 27, 470-478 (1999)) and ZAT12 (At5g59820, Meissner and Michael, Plant Mol. Biol. 33, 615-624 (1997)), which encode, respectively, an AP2-DNA binding factor and a zinc-finger protein, the functions of which are not presently known. Northern Blot analysis confirmed transcripts for RAV1 and ZAT12 accumulated within 1 hour of transferring plants to low temperature (FIG. 7A). For purposes of illustration, RAV1 and ZAT12 are used to demonstrate the invention's use of cold-regulated transcription factors. Other transcription factors, and various combinations of these transcription factors, which may or may not exhibit synergist effect, are also embodied in the present invention.

To assist in understanding these functions, Zat12 was originally identified on the basis of its homology to Epf1, a member of the two-fingered C2H2 family of transcription factors from petunia (Meissner and Michael, 1997). Epf1 has been shown to bind to a specific DNA sequence (Takatsuji et al 1994). Epf1, Zat12 and many other related transcription factors in plants contain conserved amino acid motifs in each zinc finger domain. As such it is extremely likely that, like Epf1, Zat12 is also a DNA binding protein. Furthermore, Zat12 contains an amino acid sequence with high sequence identity to the EAR (ERF-associated amphiphillic repression) motif. This motif is both necessary for the repressive function of several tobacco and Arabidopsis transcriptional repressors and sufficient to inhibit the activation function of heterologous activation domains to which it is fused (Ohta et al, 2001). A 42 amino acid fragment of Zat12 containing the EAR-related motif exhibited repression activity in a transient transactivation study indicating that ZAT12 is likely to act as DNA-binding transcriptional repressor (Hiratsu et al., 2002).

The conclusions above are supported by recent work by the inventors of the present invention that indicates over-expression in transgenic Arabidopsis of ZAT12 under the control of the strong constitutive CaMV 35S promoter, down-regulated transcript levels for at least 17 Arabidopsis genes. Ten genes were upregulated, however it is possible that some of the four down-regulated genes for which no functional information is available, are themselves transcriptional repressors of the ten upregulated genes).

RAV1 is a DNA binding protein from Arabidopsis. It contains two unrelated DNA binding domains; a B3 domain and an AP2 domain. Kagaya et al used repeated cycles of Electromobility Shift Assay (EMSA) with random 30-mer oligonucleotides and Polymerase Chain Reaction (PCR) to identify DNA sequences to which RAV1 binds. Further EMSA analysis indicates RAV1 binds to bipartite DNA sequences comprising two unrelated motifs. Thus, RAV1 is a DNA binding protein. While the AP2 and B3 domains are known to bind DNA rather than activate transcription, many plant proteins that contain either B3 domains or AP2 domains (VP1, McCarty-et al 1991, abi3,?,CBF1, Stockinger et al) have been shown to have transcriptional activatory activity. Thus, it is likely that RAV1 is also a DNA-binding transcriptional activator.

Six additional long-term up-regulated genes were found to encode known or putative transcription factors: a second putative zinc finger protein (At4g38960, Mayer et al., Nature 402, 769-777 (1999)), the R2R3-Myb transcription factor AtMYB73 (Kranz et al., Plant J. 16, 263-276 (1998)), H-protein promoter binding factor-2a (Abbaraju and Oliver, direct submission to GenBank, accession number AF079503), the HD-Zip protein AthB-12 (Lee and Chun, Plant Mol. Biol. 37, 377-384 (1998)), and two AP2-domain proteins, RAP2.7 and RAP2.1 (Okamuro et al., Proc. Natl. Acad. Sci. USA 94, 7076-7081 (1997)) (FIG. 6 and Table 1). Hierarchical clustering indicated that the expression patterns of these genes fell into four different groups (FIG. 6) suggesting they were regulated by multiple pathways. The fact that these six genes were induced after the initial wave of CBF2, RAV1 and ZAT12 induction raised the possibility that one or more of them might be induced by one of these three transcription factors. Inspection of the promoter region of RAP2.1 indicated it contained two copies of the CCGAC core sequence of the CRT/DRE elements suggesting it might be a target of the CBF activators. Indeed, Northern Blot analysis indicated the transcript levels of RAP2.1 did not increase until 4 to 8 hours after transferring plants to low temperature (FIG. 7A) and they were elevated in transgenic Arabidopsis plants that constitutively over-expressed either CBF1, CBF2 or CBF3 (FIG. 7B). Thus, the CBF regulon presumably includes a “sub-regulon” controlled by RAP2.1.

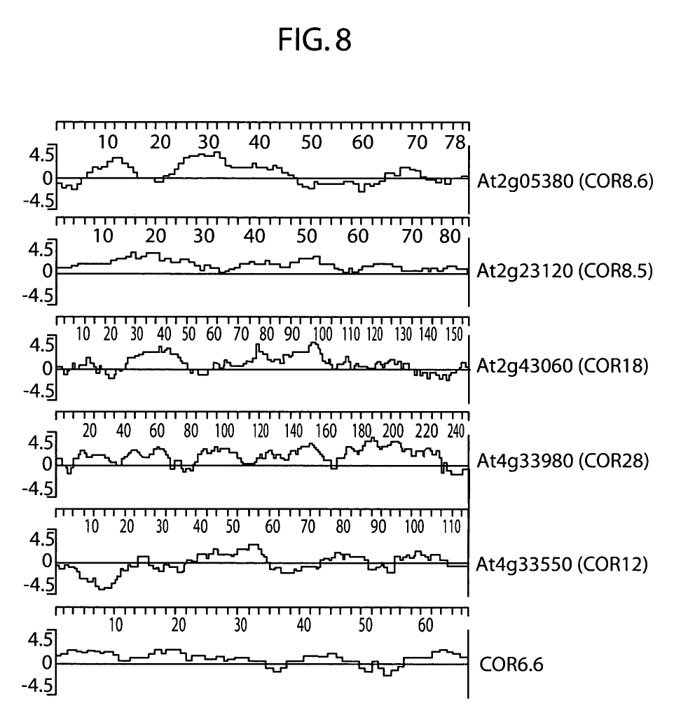

In addition to long-term up-regulated genes encoding transcription factors, there were 55 genes encoding proteins with diverse known or proposed functions (FIG. 6 and Table 1). The largest group encoded COR/LEA proteins, polypeptides thought to have roles in cryoprotection (see Thomashow, Annu. Rev. Plant Physiol. Mol. Biol. 50, 571-599 (1999)). In addition to the previously described COR6.6, COR15b, COR47, COR78 and ERD10 polypeptides was the dehydrin Xero2, a LEA3-type protein designated Di21, and a polypeptide (At1g01470) with a high degree of sequence similarity to Group 4 LEA proteins (Terryn et al., Gene 215, 11-17 (1998)). All of these COR/LEA proteins have different sequences, but have in common the biochemical property of being highly hydrophilic. Interestingly, 5 of the 11 long-term up-regulated “unknown proteins” also encoded polypeptides that were highly hydrophilic (FIG. 8). These were designated COR/LEA-like polypeptides COR8.5 (At2g23120), COR8.6 (At2g05380), COR12 (At4g33550), COR18 (At2g43060) and COR28 (At4g33980), which encode polypeptides of 8.5, 8.6, 12.3, 17.8, and 28.1 kD, respectively. The amino terminal end of COR12 is predicted to encode a signal sequence that would result in secretion of a mature hydrophilic polypeptide of 10.1 kD.

Sugars, including sucrose and raffinose, accumulate during cold acclimation in Arabidopsis (Wanner and Junttila, Plant Physiol. 120, 391-400 (1999); Gilmour et al., 2000). Thus, genes encoding proteins with roles in sugar metabolism might be expected to be cold-responsive. It has been reported that transcripts encoding sucrose synthase accumulated in response to low temperature (Gilmour et al., 2000), a finding that is confirmed here (FIG. 6 and Table 1). Additionally, transcripts for three different genes encoding putative galactinol synthases (At1g09350, At2g47180 and At1g60470), the enzyme that catalyzes the first committed step in the synthesis of raffinose, were found to accumulate in response to low temperature; in one case, induction was more than 160-fold at 24 hours (FIG. 6 and Table 1). Transcripts encoding a putative sugar transporter also accumulated in response to low temperature.

Among the most rapid and highly induced genes was that encoding ELIP1 (early light-induced protein 1) (FIG. 6 and Table 1). ELIP1, as well as ELIP2, are nuclear encoded thylakoid membrane proteins that are expressed in response to light stress (Moscovivi-Kadouri and Chamovitz, Plant Physiol. 115, 1287 (1997); Heddad and Adamska, Proc. Natl. Acad. USA 97, 3741-3746 (2000)). They are thought to be photoprotective pigment carriers or chlorophyll exchange proteins (Adamska, Plant Physiol. 100, 794-805 (1997)). Expression of this gene indicates the plants were potentially experiencing light stress.

Finally, it was found that transcripts for GIGANTEA (GI) were found to increase in some five to ten-fold in response to low temperature (FIG. 6 and Table 1). The GI gene encodes a protein with no homology to any proteins of known function in the databases (Fowler et al., EMBO J. 18, 4679-4688 (1999); Park et al., Science 285, 1579-1582 (1999)). While the function of the GI protein is unknown, mutations in the GI gene cause a plieotropic phenotype with effects on flowering in response to photoperiod, phyB signaling, the circadian clock and carbohydrate metabolism (Koornneef et al., Mol. Gen. Genet. 229, 57-66 (1991); Eimert et al., Plant Cell 7, 1703-1712 (1995); Fowler et al., 1999; Park et al., 1999; Huq et al., Proc. Natl. Acad. Sci. USA 97, 9789-9794 (2000)), but no association with cold acclimation has previously been-reported.

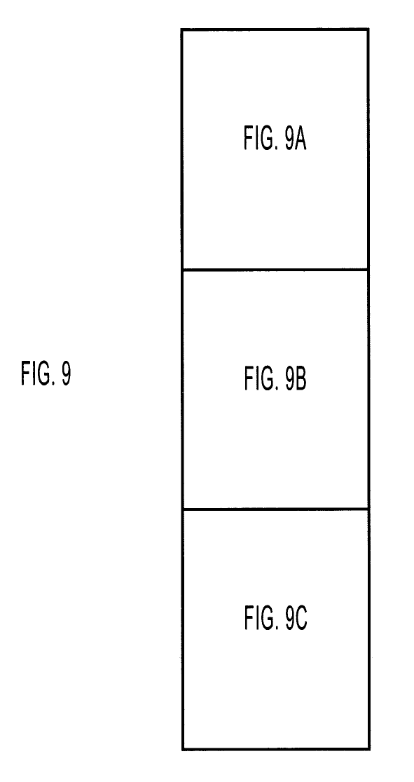

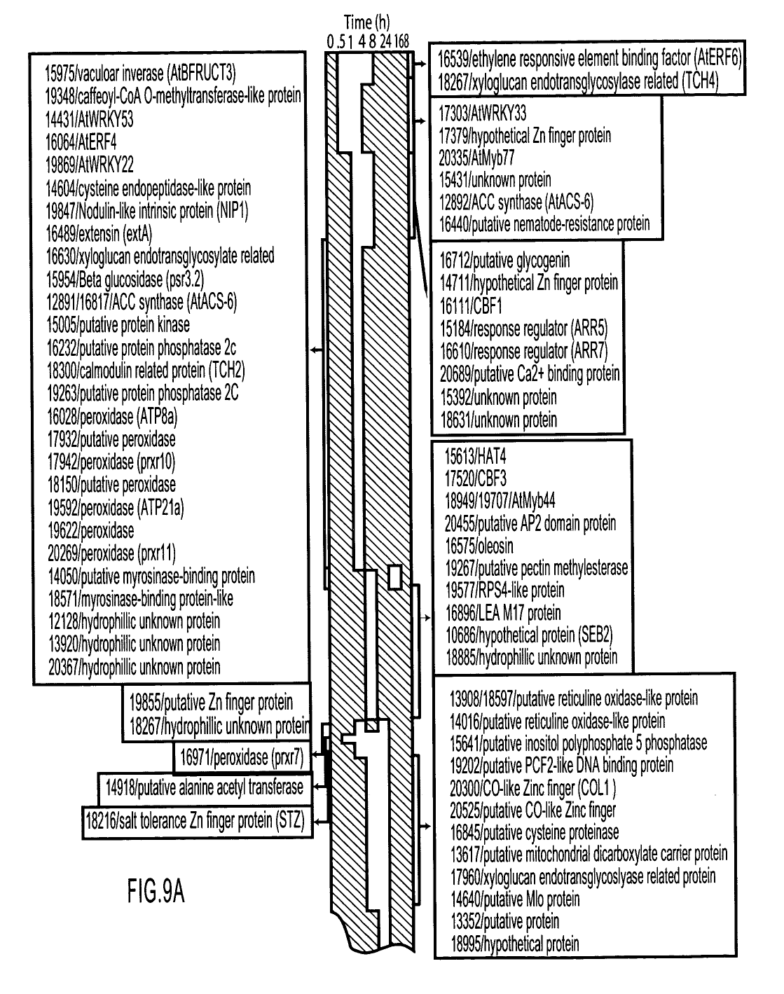

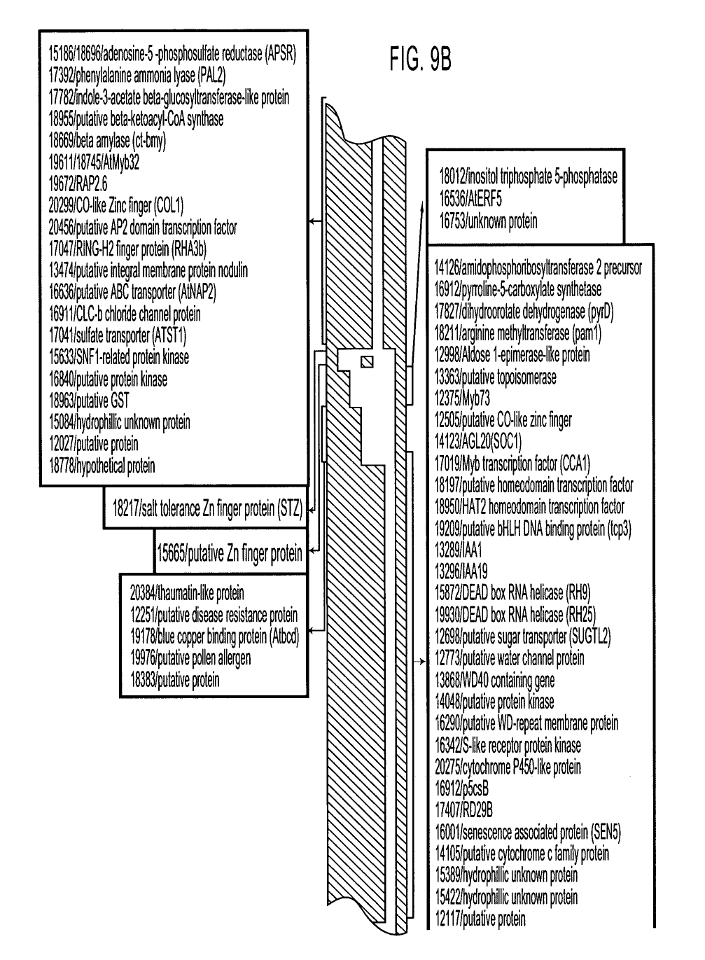

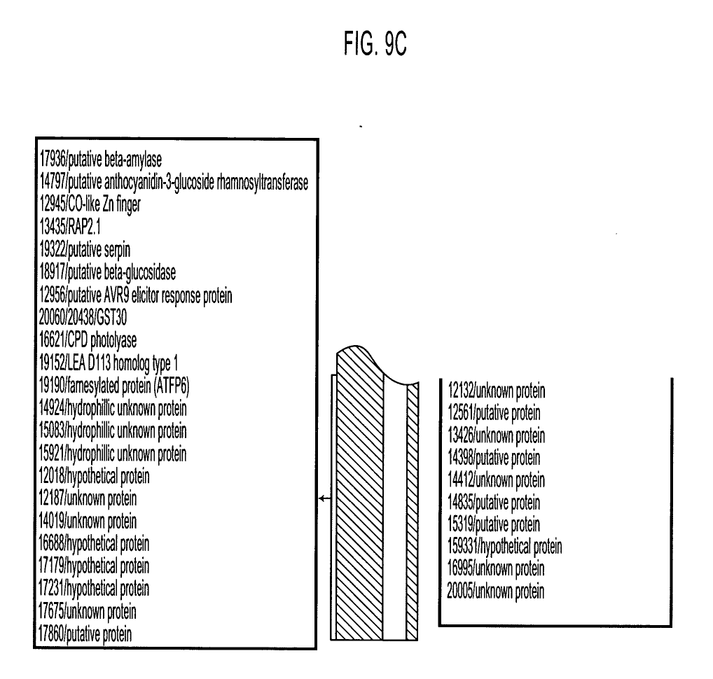

Of the 306 genes designated as cold-responsive, 156 (51 percent) were up-regulated transiently in response to low temperature (FIGS. 5B and 9 and Table 2). Hierarchical clustering of these genes revealed transfer of plants from warm to cold temperature set off a series of transient waves of changes in the transcriptome (FIGS. 5B and 9). At each time-point, new genes were up-regulated, and in most cases, only remained induced for one or two of the time-points. As with the long-term cold-responsive genes, this pattern was more complex than the two step CBF cold-response pathway, suggesting multiple regulatory systems were involved in the response to temperature downshift. Indeed, of the 156 transiently cold-induced genes, 34 (22 percent) corresponded to known or putative transcription factors (FIG. 9 and Table 2). Additionally, 16 genes (10%) encoded known or putative proteins involved in signal transduction or cellular communication including response regulators, protein kinases and phosphatases. In total, about 33 percent of the transiently expressed genes potentially had roles in gene regulation. The transient nature of the changes in transcript levels suggested the abrupt lowering of temperature might have resulted in a short-lived “shock” response followed by an adjustment to the new environmental conditions. Indeed, low temperature may cause a decrease in the turnover rate of photosystem (PS) II components causing an increase in PS II excitation pressure or “excess excitation energy” and the generation of damaging ROS including hydrogen peroxide (see Huner et al, Trends Plant Sci. 3, 224-230 (1998)). An indication that such a response occurred in experiments relating to the present invention was that among the transiently expressed genes were three known or putative glutathione S-transferases that are known to be involved in the detoxification of toxic metabolites arising from oxidative damage caused by excess excitation energy (see Marr, Plant Mol. Biol. 47, 127-158 (1996)) and nine known or putative peroxidases that potentially also contribute to detoxification of hydrogen peroxide (Østergaard et al., FEBS Lett. 433, 98-102 (1998)). Moreover, seven genes recently shown to be induced by hydrogen peroxide in Arabidopsis (Desikan et al., Plant Physiol. 127, 159-172 (2001)) were among the genes found to be transiently induced by cold. These genes encoded a blue copper-binding protein, adenosine-5-phosphosulfate reductase, a putative zinc-finger transcription factor (At5g04340), AtERF-4, CCA1, a putative nematode resistance protein and a protein of unknown function (At2g36220).

The production of ethylene has also been associated with cold-stress (Ciardi et al., Plant Physiol. 101, 333-340 (1997); Morgan and Drew, Plant Physiol. 100, 620-630 (1997); Yu et al., Plant Physiol. 126, 1232-1240 (2001)). In this regard, it was notable that genes involved in ethylene signaling were among those rapidly induced with low temperature (FIG. 9 and Table 2). Within the first hour of transfer, transcripts accumulated for ACC synthase (AtACS-6), which catalyzes a limiting step in ethylene synthesis, and two ethylene-responsive transcription factors, AtERF-4 and AtERF-5 (Fujimoto et al., Plant Cell 12, 393-404 (2000)). However, two other known ethylene-inducible genes, AtERF-1 and basic chitinase were not found to be induced (these genes are represented by probe sets on the GeneChip). This, and the fact that cold-induction of AtERF-4 and AtERF-5 can occur independent of ethylene (Fujimoto et al., 2000) may lead to the conclusion that although rapid transfer of plants to low temperature resulted in a burst of transcript accumulation for ACC synthase, little, if any, ethylene was actually produced.

Of the total transiently-expressed genes, 24 percent encoded “unknown proteins” (FIG. 9 and Table 2). Of these, 11 were novel polypeptides that, like LEA/COR proteins, were unusually hydrophilic. The probe sets corresponding to these genes were At2g41010, At1g23710, At2g28400, At4g35320, At2g22860, At4g04330, At1g10410, At2g46970, At2g32210, At2g36220 and At1g11960 (FIG. 9). Additionally, transcript levels for genes encoding three previously described LEA/COR proteins, namely LEA M17, LEA D113 and RD29B, were found to be transiently expressed (FIG. 9 and Table 2). These results indicated low temperature generated a short-lived signal that induces expression of LEA/COR and LEA/COR-like genes through a pathway that is independent of the CBF cold-response pathway. Of the 306 genes scored as being cold-responsive, 88 (27 percent) corresponded to down-regulated genes, 46 of which were transiently affected and 42 of which were affected “long-term” (i.e., they were down-regulated 2.5-fold or more at 7 days) (FIG. 2 and Table 3). This is the first known indication that extensive down-regulation of gene expression occurs in response to low temperature.

Like the genes that were up-regulated in response to low temperature, the down-regulated genes encoded proteins with a wide range of functions including transcription, signaling, cell wall biogenesis and defense. Four of the long-term down-regulated transcripts encoded proteins with known or predicted roles in energy production: two light harvesting proteins, Lhca2*1 and Lhcb4*3; a putative photosystem II 5 kD protein; and a ferredoxin precursor. The down regulation of these genes may have resulted from the decreased light levels that the plants were exposed to during the cold treatment. However, it has been reported that transfer of warm-grown plants to chilling temperatures (4° C.) leads to a rapid inhibition of photosynthesis followed by a reduction in transcript levels for genes encoding photosynthetic proteins (Krapp and Stitt, Planta 195, 313-323 (1995); Strand et al., Plant J. 12, 605-614 (1997)). This effect of low temperature may have also caused the decrease in transcript levels of photosynthesis-related proteins observed here.

A striking difference between the down- and up-regulated genes was that few genes were down-regulated during the first four hours of exposing plants to cold temperature. The full significance of this apparent delay is uncertain as any decrease in transcript levels must take into account the turnover rate of the transcript. Given the half life of the average plant transcript is on the order of several hours (Abler and Green, Plant Mol. Biol. 32, 63-78 (1996)), one would not expect many changes until after 4 hours. However, most (68 percent) of the changes did not occur until the 24 hour time-point or after indicating that even when the turnover rate of transcripts is taken into account, the mechanisms that lead to down-regulation of transcript levels in response to cold treatment is delayed compared to that which leads to up-regulation.

Many of the findings described above indicated that regulatory pathways, in addition to the CBF cold-response pathway, are activated in response to low temperature. To explore this issue further, we profiled the transcriptomes of warm-grown transgenic Arabidopsis plants that constitutively expressed either CBF1, 2 or 3 and compared them to the profiles of control plants. Fold-change values were calculated by comparing the data for each transgenic line against two control samples thus generating six comparisons. A gene was designated as being a member of the CBF regulon if the signal intensity was above background (“present”) for all three transgenic lines, if there was a difference call of “increase” for all six comparisons, and if the fold-increase value was greater than or equal to three for all six comparisons. For a gene to be designated as being cold-responsive, but independent of the CBF cold-response pathway, it had to be up-regulated by cold treatment, but could not be up-regulated in any of the CBF over-expressing plants; i.e., the probe sets had to be assigned a difference call of “no change” in each of the six comparisons between the three CBF transgenic lines and two individual wild type samples. These criteria were stringent and consequently, 128 (59 percent) of the genes up-regulated by cold could not be assigned to either of the categories. However, for 90 genes an assignment could be made.

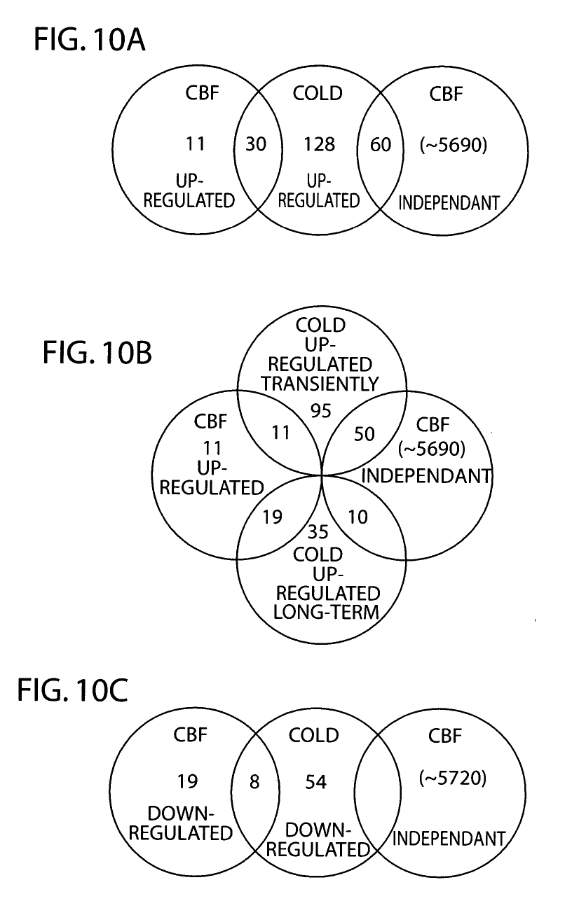

Of the cold-induced genes, 60 were not up-regulated in any of the CBF-expressing plants and thus, were designated as being independent of the CBF cold-response pathway (FIG. 10A). Of these, the large majority, 50 genes (or 83 percent), were transiently up-regulated upon exposure to low temperature; the remaining 10 (17 percent) were long-term up-regulated (FIG. 10B).

Analysis of the CBF-expressing plants indicated that 41 genes were up-regulated in all three transgenic lines (FIG. 10A and Table 4). Included in this group were genes previously reported as being CBF-targets such as COR6.6, COR78, COR47, P5CSb and ERD10 (Jaglo-Ottosen et al., 1998; Gilmour et al., 2000; Seki et al., 2001). Of the 41 CBF-regulated genes, 30 were found to be up-regulated in response to low temperature in the experiments described above (FIG. 10A; Tables 1, 2 and 3). These 30 genes were thus considered genuine members of the CBF regulon. The 11 “CBF-responsive” genes that were not cold-induced were presumably “down-stream” consequences of CBF expression related to the altered growth phenotypes displayed by plants over-expressing CBF (Liu et al., 1998; Kasuga et al., 1999; Gilmour et al., 2000). Of the 30 genes that were members of the CBF regulon, 19 were in the long-term cold-responsive group and the remaining 11 were induced transiently (FIG. 10B). Among these genes was RAP2.6, which encodes an AP2 protein and thus is another likely transcription factor that controls expression of a sub-regulon of the CBF regulon.

TABLE 4
Genes up-regulated by over-expression of CBF1, CBF2 and CBF3.
Genes up-regulated by constitutive expression of CBF1, CBF2 and CBF3

| | | | Transgenic line | | | |
|---|---|---|---|---|---|---|
| Probe set | AGI Identifier | Description | CBF1 | CBF2 | CBF3 | Cold-responsive |
| Metabolism | | | | | | |
| 12532_at | At1g10760 | putative pyruvate phosphate dikinase | 4 | 3.1 | 4.2 | |
| 13018_at | At1g09350 | putative galactinol synthase | 88.4 | 52.9 | 79.2 | x |
| 14832_at | At4g23600 | tyrosine transaminase like protein | 17.7 | 7.7 | 8.3 | |
| 14847_at | At1g60470 | putative galactinol synthase | 17.7 | 4.0 | 8.3 | x |
| 16192_at | At2g24560 | putative GDSL lipase/hydrolase | 32.4 | 15.9 | 14.1 | x |
| 16912_at | At3g55610 | pyrroline-5-carboxlyate synthetase (P5CS2) | 7.5 | 4 | 6.9 | x |
| 18596_at | At1g62570 | putative glutamine synthase | 42.0 | 28.4 | 30.8 | x |
| Transcription | | | | | | |
| 19672_at | At1g43160 | AP2 domain protein (RAP2.6) | 7.6 | 14.3 | 4.0 | x |
| 20471_at | At1g46768 | AP2 domain protein (RAP2.1) | 16.0 | 16.1 | 15.2 | x |
| Protein fate | | | | | | |
| 19322_at | At1g47710 | putative serpin | 5.7 | 3.7 | 3.6 | x |
| Transport facilitation | | | | | | |
| 20149_at | At1g08890 | putative sugar transporter | 8.0 | 7.1 | 6.9 | x |
| 13950_at | At4g17550 | glycerol-3-phosphate permease-like protein | 11.9 | 4.4 | 6.5 | x |
| 14990_at | At2g16990 | putative tetracycline transporter protein | 15.4 | 7.6 | 12.6 | |
| Cellular biogenesis | | | | | | |
| 15695_s_at | At2g18050 | histone H1-3 | 6.1 | 4.3 | 10.9 | |
| 12773_at | At2g28900 | putative water channel protein | 4.9 | 4.8 | 5.3 | x |
| 12606_at | At4g04020 | putative fibrillin | 7.2 | 4.0 | 4.8 | |
| 17963_at | At4g12470 | pEARLI 1-like protein | 7.1 | 8.9 | 7 | x |
| Cell rescue, defence, | | | | | | |
| cell death and aging | | | | | | |
| 12956_i_at | At1g05170 | putative Avr9 elicitor response protein | 13.0 | 6.3 | 9.2 | x |
| 13004_at | At2g17840 | putative senescence-associated protein 12 | 6.4 | 3.4 | 5.6 | x |
| 13225_s_at | At1g20440 | dehydrin (COR47) | 12.4 | 12.9 | 14.2 | x |
| 13785_at | At2g42530 | COR15b | 54.3 | 55.6 | 61.9 | x |
| 15103_s_at | At1g20450 | dehydrin (ERD10) | 19.2 | 16.9 | 16.8 | x |
| 15611_s_at | At5g52310 | COR78 | 89.4 | 86.4 | 92.5 | x |
| 15997_s_at | At1g20440 | dehydrin (COR47) | 11.3 | 10.5 | 11.4 | x |
| 16450_s_at | At3g50980 | dehydrin (Xero 1) | 5.8 | 6.3 | 8.4 | |
| 16943_s_at | At4g15910 | Di21 | 18 | 4.6 | 13.6 | x |
| 17407_s_at | At5g52300 | RD29B | 30.8 | 7.8 | 23.8 | x |
| 18594_at | At1g01470 | LEA protein | 10.4 | 7.5 | 9.2 | x |
| 18699_i_at | At5g15970 | COR6.6 | 17.9 | 20.3 | 19.4 | x |
| 18700_r_at | At5g15970 | COR6.6 | 13 | 15.2 | 13.3 | x |
| 18701_s_at | At5g15970 | COR6.6 | 26.6 | 32.9 | 31.7 | x |
| 18928_at | At2g43620 | putative endochitinase | 52.1 | 55.1 | 30.6 | x |
| 19186_s_at | At3g50970 | dehydrin (Xero 2) | 76 | 73.3 | 84.3 | x |
| Unknown role | | | | | | |
| 15073_at | At2g01890 | putative purple acid phosphatase | 4.4 | 4.4 | 4.7 | |
| unknown protein | | | | | | |
| 12018_at | At1g64890 | hypothetical protein | 6.7 | 4.6 | 5.5 | x |
| 12175_at | At2g02180 | unknown protein | 4.5 | 3.3 | 3.8 | |
| 12767_at | At2g23120 | unknown protein (COR8.5) | 6.0 | 5.6 | 6.4 | x |
| 13900_s_at | At3g47380 | putative protein | 4.4 | 8.1 | 3.9 | |
| 14398_at | At4g21570 | putative protein | 9.7 | 5.1 | 6.0 | x |
| 15053_at | At2g41870 | unknown protein | 6.7 | 4.4 | 7.0 | |
| 16688_at | At1g10080 | hypothetical protein | 8.5 | 5 | 6.1 | x |
| 17864_at | At2g24260 | unknown protein | 6.7 | 3.9 | 5.7 | |
| 18383_at | At1g04570 | putative protein | 12.0 | 3.2 | 6.5 | x |
| 19368_at | At3g27330 | unknown protein | 24.4 | 14.7 | 22 | x |

Mean fold-change values for the comparisons between each transgenic line and the two wild type samples are shown. Probe set names in boldface correspond to genes previously reported as up-regulated by CBF overexpression. An “X” in the cold-responsive column indicates that a probe set was identified as up-regulated by cold

Finally, of the 88 genes found to be down-regulated during cold acclimation, eight were also down-regulated by CBF over-expression: At4g22690, At3g57260, At1g75040, At2g14560, At1g21270, At2g43570, At1g69490 and At4g14400 (FIG. 10C and Table 4). These results indicate for the first known time that the CBF cold responsive pathway not only acts to activate gene expression, but is also involved in repressing the expression of certain genes.

Fifteen of the 19 genes that Seki et al (2001) identified as being cold-inducible were represented by probe sets on the AFFYMETRIX GENECHIPS used in the present experiments. Six of these genes, COR47, COR78, COR6.6, ERD10, SEN12/ERD7 and CBF3/DREB1a, had previously been reported to be cold inducible and were identified as cold-regulated in these experiments. One of the nine novel cold-regulated genes identified by Seki et al. (2001), that encodes a β-amylase (FL590/At4g17090), was also found by us to be cold-regulated. Four additional novel cold-regulated genes identified by Seki et al. (2001) were up-regulated by cold in our experiments (were given a difference call of increase), but were not above our 3-fold cut-off, and thus were not designated as being cold-inducible. These genes, which were up-regulated between 2 and 3-fold in the Seki et al. (2001) experiments, encoded a putative cold acclimation protein (FL3-5A3/At2g15970), a nodulin-like protein (FL5-1A9/At4g27520), ferritin (FL5-3A15/At5g01600) and a homolog of DC1.2 (FL5-2122/At5g62350). Finally, four of the genes designated as being cold-regulated by Seki et al. (2001) were not cold-regulated in the experiments. These genes, which were induced between 2 and 3-fold, encoded a homolog of LEA protein SAG21 (FL5-3M24/At4g02380), a rice glyoxalase homolog (FL595/At1g11840), a DEAD box ATPase/RNA helicase (FL25A4/At3g01540) and EXGT-A2 (FL5-3P12/At1g14720). The reason for this difference is not known but could reflect differences in plant culturing conditions, environmental treatments, or differences in the expression profiling methods used.

Previous studies have established the CBF cold-response pathway is an integral component of the cold acclimation response (see Shinozaki and Yamaguchi-Shinozaki, 2000; Thomashow, 2001). Additional cold-regulatory pathways might also have important roles in cold tolerance contributing to increased freezing tolerance as well as mediating physiological, biochemical and structural changes required for growth and development at low temperature. Xin and Browse (Proc. Natl. Acad. Sci. USA 95, 7799-7804 (1998)) described a mutant of Arabidopsis, designated eskimol, which is constitutively freezing tolerant, but does not express known members of the CBF regulon. Here, the results provide direct evidence for cold-regulatory pathways in addition to the CBF cold-response pathway. Of the 306 cold-responsive genes, 106 were affected on the long term; i.e., transcript levels were up- or down-regulated at least 2.5-fold at 7 days. Among these genes were members of the CBF cold-response pathway. However, in addition, eight other genes encoding known or putative transcription factors were found to be long-term cold-responsive: ZAT12, RAV1, AtMYB73, ATHB-12, H-protein binding factor-2-a, RAP2.1, a zinc finger protein (At4g38960) and RAP2.7 (FIG. 6 and Table 2). One of these, RAP2.1, proved to be a target of the CBF transcription factors (FIG. 7) and thus, presumably regulates expression of a sub-regulon of genes within the larger CBF regulon. However, two of the transcription factors, ZAT12 and RAV1, were found to be induced in parallel with the CBF transcriptional activators (i.e., transcript levels were increased more than three-fold at 30 minutes). It is possible that low temperature regulation of ZAT12 and RAV1 involves action of the same regulatory proteins that activate CBF expression. Gilmour et al. (1998) previously speculated on the existence of such a “super regulon” controlled by a hypothetical protein ICE (Inducer of CBF Expression). Regardless, the parallel induction of CBF2, ZAT12 and RAV1, well before induction of known CBF-target genes such as COR47, COR6.6 and COR78, argues against their being members of the CBF regulon. Consistent with this notion was that ZAT12 transcript levels were unaffected in either CBF1, 2 or 3 over-expressing plants. A comparison of the transcriptomes of CBF overexpressing plants with control plants indicate that at least 60 cold-induced genes are independent of the CBF cold-response pathway including the expression of at least 15 transcription factors.

Investigations to date have focused on studying genes that are up-regulated in response to low temperature. The results presented here, however, indicate that down-regulation of gene expression may also be an important component of adapting to low temperature. Indeed, the down-regulation response was extensive; transcript levels for 88 genes were found to decrease either transiently or long term in response to low temperature. These genes, like those that were up-regulated, encompassed a wide range of functions including transcription, signaling, cell wall biogenesis, defense, and photosynthesis, almost all of which (to the best of our knowledge) have not been previously shown to be “cold-repressed.” Some of the changes in transcript levels, especially those associated with photosynthesis, might have been due to the lower light conditions used during the cold treatment. However, this would not appear to be true for all of the down-regulated genes. In particular, eight genes down-regulated in response to low temperature were also down-regulated at warm temperature in response to constitutive expression of CBF1, CBF2 and CBF3. These data indicate the CBF cold-response pathway includes down-regulating the expression of certain genes and point to genes whose expression might be incompatible with enhancement of freezing tolerance. Previous studies have identified 14 genes as being members of the CBF regulon. Here, it is expanded to 45 (37 up-regulated and 8 down-regulated). Among the newly described members of the CBF regulon are genes encoding two putative transcription factors, RAP2.1 and RAP2.6 that presumably control expression of sub-regulons of the larger CBF regulon. In this regard, it is interesting to note that a number of the genes activated by CBF expression do not have the core CCGAC sequence of the CRT/DRE-element within 1 kb of the start of transcription. These, thus, are candidates for being members of a CBF sub-regulon controlled by RAP2.1, RAP2.6 or an as yet to be-discovered transcription factor(s) induced by the CBF activators. Other new members of the CBF regulon include genes encoding a putative sugar transporter, water channel proteins and a new hydrophilic polypeptide that potentially acts as a cryoprotectant. Additionally, three genes are putatively encoding galactinol synthase, which catalyzes the first committed step in raffinose synthesis. In fact, Taji et al. (Plant J. 29, 417-426 (2002)) has reported three Arabidopsis genes that encode proteins with galactinol synthase activity and that one of these, AtGolS3 (which corresponds to probe set 18596_at), was induced in response to low temperature and overexpression of CBF3/DREB1a. Given that the present experiments sampled about a third of the genome, there could be as many as 100 genes that are part of the CBF regulon. This, again, is probably an underestimate as the criteria used here to assign a gene to the CBF regulon were that it had to be induced 3-fold or more in each CBF-overexpression line. It is not known at present, however, whether CBF1, 2 and 3 control completely overlapping sets of genes. Differences in the set of genes that were up-regulated in the CBF1, 2 and 3 transgenic plants tested here were observed. However, it is not known whether these differences were due to biological variation, differences in the level of CBF expression, or reflected bona fide differences in the activities of the CBF proteins.

One intriguing finding is that among the 280 newly described cold-responsive genes was GI. GI transcript levels increased five to eight-fold after 24 hours of cold treatment and remained elevated after 7 days. GI is a novel protein with roles in the promotion of flowering by photoperiod and circadian clock function (Fowler et al., 1999; Park et al., 1999), but has not previously been associated with acclimation to low temperature. Interestingly, however, it is known that in addition to entrainment by light/dark cycles, the circadian clock in plants may be entrained by temperature cycles (Kloppstech et al., 1991; Beator et al., Plant Physiol. 100, 1780-1786 (1992); Heintzen et al., Plant J. 5, 799-813 (1994); McWatters et al., Nature 408, 716-720 (2000)). Park et al. (1999) proposed that GI functions in a light input pathway to the clock. The observation that GI is cold-responsive raises the possibility that GI might function in input pathways to the clock that transmit both light and temperature signals.

More than half of the cold-responsive genes were upor down-regulated transiently in response to low temperature. It seems likely that the expression of a significant number of these genes was due to the abrupt change in temperature used in our experiments; plants were directly transferred from 22° C. to 4° C., a protocol that is commonly used in studying cold acclimation. Transferring plants from warm to cold temperature in the light (as was the case in the present experiments) may result in excess light energy that may lead to the production of hydrogen peroxide and other reactive oxygen species (see Huner et al, 1998). This, in turn, may lead to the induction of genes involved in protecting cells against oxidative stress. A number of genes known to be responsive to hydrogen peroxide were transiently up-regulated in our experiments, including glutathione S-transferases, indicating the plants were at least transiently experiencing oxidative stress. Thus, some of the transient cold-responsive genes probably do not have direct roles in life at low temperature per se, but instead have critical roles in enabling plants to adjust to quickly fluctuating environmental conditions including protection against conditions that result in excess light (Huner et al., 1998). It would not be appropriate, however, to draw the conclusion that none of the transiently expressed genes have roles in freezing tolerance or other fundamental aspects of life at low temperature. CBF1 and CBF3 were among the transiently expressed genes and thus inclusion on this list certainly can not be used to dismiss potential importance in cold acclimation. It is also true that arbitrary criteria were use to place genes into the “transient” category; their transcript levels had to have returned to within 2.5-fold that of the warm sample after 7 days of cold-treatment. However, the transcript levels for about 55 percent of these genes were elevated 2.5-fold or more at 24 hours so it is possible that many of them remained actively up-regulated for several days after transfer to the cold. Moreover, the levels of the proteins for these genes could have remained elevated for many days after transfer to cold. Finally, it is relevant to note that when Escherichia coli (and other bacteria) are subjected to an abrupt temperature drop of about 15° C., they undergo a “cold-shock” response (see Yamanaka, J. Mol. Microbiol. Biotechnol. 1, 193-202 (1999)) that includes the transient induction of genes, some of which (e.g., the Csp gene family) have been shown to have critical roles in enabling the bacteria to grow at low temperature (Xia et al., Mol. Microbiol. 40, 179-188 (2001)). Methods

The results of this transcriptome study demonstrate in unparalleled fashion the highly complex nature of plant adaptation to low temperature. The results indicate that the expression of hundreds of genes are affected upon exposing plants to low temperature and that this involves the action of multiple cold-regulatory pathways including the activation of regulons within regulons. Through the further application of genomic approaches, it should ultimately be possible to construct a diagram of the low temperature “gene circuitry” in plants and determine the roles of the regulatory networks in cold tolerance.

Plant Material and Growth Conditions: Arabidopsis (L.) Heynh. ecotype Wassilewskija (Ws)-2 and transgenic plants constitutively expressing CBF1 (G5, G6, G26), CBF2 (E2, E8, E24; S. J. Gilmour and M. F. Thomashow, unpublished results) or CBF3 (A28, A30, A40; Gilmour et al., 2000) in the Ws-2 background, were used in these experiments. Seeds were surface-sterilized then spread on Petri plates containing Gamborg's B-5 medium (Life Technologies Inc., Gaithersburg, Md.) solidified with 0.8 percent phytagar (Life Technologies Inc., Gaithersburg, Md.). Immediately after plating, the seeds were stratified for 4 days at 4° C. to ensure uniform germination. Plants were grown in controlled environment chambers at 22° C. under continuous illumination of 100 μmol m⁻² s⁻¹ from cool-white fluorescent lights for 11 days. For cold treatments, plates containing the plants were transferred to 4° C. under continuous light (20-40. μmol m⁻² s⁻¹) as described (Gilmour et al., 1998) and tissue samples were harvested after 0.5 h, 1 h, 4 h, 8 h, 24 h and 7 d. Duplicate samples for each cold time-point and single samples from each of the CBF transgenic lines were harvested for profiling.

RNA isolation and probe labeling: The aerial parts of 50-150 plants grown on a single plate, were pooled for each RNA sample. Total RNA was extracted from the samples using the QIAGEN RNEASY PLANT KIT (Qiagen Inc., Valencia, Calif.). Biotinylated target RNA was prepared from 16 μg of total RNA using the procedure outlined by the manufacturer of the Arabidopsis GENECHIP (Affymetrix Inc., Santa Clara, Calif.). Briefly, a primer encoding a T7 RNA polymerase promoter fused to (dT)₂₄ (Genset Oligos, La Jolla, Calif.) was used to prime double-stranded cDNA synthesis using the SUPERSCRIPT CHOICE SYSTEM (Life Technologies, Gaithersburg, Md.). The resulting cDNA was transcribed in vitro using the BioArray High Yield RNA TRANSCRIPT LABELING KIT (Enzo Biochem Inc., New York, N.Y.) in the presence of biotinylated UTP and CTP to produce biotinylated target cRNA.

Affymetrix GENECHIP hybridization and data collection: The labeled target cRNA was purified, fragmented and hybridized to Arabidopsis Genome GENECHIP arrays according to protocols provided by the manufacturer (Affymetrix Inc., Santa Clara, Calif.) in a Hybridization Oven model 640 (Affymetrix Inc., Santa Clara, Calif.). The GENECHIPs were washed and stained with streptavidin-phycoerythrin using a GENECHIP Fluidics Station model 400 then scanned with a Gene Array Scanner (Hewlett-Packard, Palo Alto, Calif.).

Data Analysis: The Microarray Suite 4.0 and Data Mining Tool 1.0 (Affymetrix Inc., Santa Clara, Calif.) software packages were used for the analysis of microarray data. The output from all GeneChip hybridizations was globally scaled such that its average intensity was equal to an arbitrary target intensity of 100. Since all experiments were scaled to the same target intensity, this allowed comparison between GeneChips. The mean noise for the GeneChips used in these analyses was 3.7+/−3.5. Average difference (gene expression) and fold-change values were calculated from the GeneChip fluorescent intensity data. The software was also used to determine whether expression of each gene was “present” or “absent” (absolute call) and whether the fold-change value represented a genuine change in expression (difference call). Fold-change values were calculated for each sample harvested at 4° C. compared to each of the samples harvested before transfer to 4° C. generating four measurements for each gene at each time-point during the cold treatment. Fold-change values were also calculated for the three CBF transgenic samples compared to each of the wild type samples generating six measurements for each gene.

Probe sets that met the following criteria were selected for further analysis. Those determined to be up-regulated by the cold treatment were selected as having, at any time-point, an absolute call of present in both cold samples and difference calls of increase and a fold-changes of at least 3.0 for all four fold-change comparisons. Those determined to be down-regulated by the cold treatment were selected as having an absolute call of present in both warm samples and, at any time-point, difference calls of decrease paired with fold-changes of at least −3.0 for all four fold-change comparisons. Cold-regulated genes were determined to be long-term up-regulated if at the 7 day time-point, both absolute calls were present and the four fold-change comparisons were 2.5-fold or greater associated with four difference calls of increase. Similarly, cold-regulated genes were determined to be long-term down-regulated if at the 7 day time-point, the four fold-change comparisons were −2.5-fold or greater associated with four difference calls of decrease. Genes up-regulated by CBF over-expression were selected as having an absolute call of present in all three CBF transgenic samples and difference calls of increase and a fold-changes of at least 3.0 for all six fold-change comparisons between wild type and the CBF transgenic lines. Those determined to be down-regulated by CBF expression were selected as having an absolute call of present in both wild type samples and difference calls of decrease paired with fold-changes of at least −3.0 for all six fold-change comparisons between wild type and the CBF transgenic lines. Cold-regulated genes, which were independent of CBF expression, were selected as having a difference call of “no change” in all six fold-change comparisons between wild type and the CBF transgenic lines.

MICROSOFT Access database management software (Microsoft Corp. Redmond, Wash.) was used to manage and filter the GeneChip data and Genespring 4.0.4 (Silicon Genetics, Redwood City, Calif.) was used for generating hierarchical gene clusters using a Pearson correlation (separation ratio 0.5, minimum distance 0.001). Before clustering all data points, which were associated with a difference call of no change, were converted to 1. “Binary” hierarchical clusters were generated by altering the data used to generate the clusters such that data points that fulfilled a particular set of criteria were converted to 2 while all other points were assigned a value of 1.

The false positive rate was calculated as the number of probe sets significantly changed as a percentage of probe sets on the array (Lipschutz et al., Nat. Genet. 21, 20-24 (1999)). A single total RNA sample was used to prepare two cDNA samples and subsequently cRNA samples that were then hybridized to two different GeneChips and fold-change values calculated. Genes were counted as false changes if they showed changes of three-fold or greater associated with a difference call of increase and a signal threshold above background (present) in at least one sample of the comparison.

Northern Hybridization Analysis: Total RNA samples (10 μg), isolated as described above, were electrophoresed through agarose gels and northern transfers were prepared and hybridized as described in Hajela et al. (Plant Physoil. 93, 1246-1252 (1990)) using high stringency wash conditions (Stockinger et al., 1997). Gene specific probes to RAV1, RAP2.1, and ZAT12 were obtained by amplifying approximately 1 kb of the coding region of these genes from Arabidopsis genomic DNA The primers used were:

| | |
|---|---|
| RAV1, | 5′-TCTAGACGAAAAAGTCGTCGGTAGGT-3′ and |
| | 5′-GGATCCGAGTTGTTACGAGGCGTGAA-3′; |
| ZAT12, | 5′-ACTAGTCAGAAGAAAAATGGTTGCGATA-3′ and |
| | 5′-GGATCCGAAAAATTCAAAGAATGAGAGAAACA-3′; |
| RAP2.1, | 5′-TCTAGATCAATGGAAAGAGAACAAGAA-3′ and |
| | 5′-AGATCTAAATTGACTATATATCTCCGGATTC-3′. |

The probes were radiolabeled with ³²P by priming with random octomers (Invitrogen, Carlsbad, Calif.).

Applications

Plants modified to enhance stress tolerance: The present invention also provides a method for recombinant engineered plants with a new or altered response to one or more environmental stresses.

According to one embodiment, a copy of a gene native to a plant that encodes a binding protein according to the present invention is recombinantly introduced into the plant such that the plant expresses a recombinant binding protein encoded by the recombinant copy of the gene.

According to another embodiment, a non-native gene that encodes a binding protein according to the present invention is recombinantly introduced into a plant such that the plant expresses a recombinant binding protein encoded by the recombinant non-native gene.

According to yet another embodiment, a native or normative DNA regulatory sequence is recombinantly introduced into a plant such that the recombinant DNA regulatory sequence regulates the expression of one or more environmental stress tolerance genes in the plant. The plant includes a gene that encodes a binding protein capable of binding to the recombinant DNA regulatory sequence.

In yet another embodiment, a native or non-native promoter is recombinantly introduced into a plant such that the recombinant promoter regulates the expression of a binding protein which binds to a DNA regulatory sequence.

According to each of the above embodiments, unless otherwise specified, the gene encoding the binding protein, the promoter promoting the expression of the binding protein, the DNA regulatory sequence, and the environmental stress tolerance genes may be non-recombinant or recombinant sequences. The recombinant sequences may be native to the plant or may be non-native to the plant. All the above permutations are intended to fall within the scope of the present invention.

In each of the above embodiments, expression of the recombinant copy of the regulatory gene may be under the control of a promoter. The promoter may be recombinant or non-recombinant. In the case of recombinant promoters, the promoter may be native or non-native to the plant.

When a recombinant promoter is used, the promoter may be selected to cause expression of the binding protein in a manner that is different than how the binding protein is expressed by the plant in its native state. For example, the promoter may increase the level at which the binding protein is expressed, express the binding protein without being induced by an environmental stress and/or express the binding protein in response to a different form or degree of environmental stress than would otherwise be needed to induce expression of the binding protein. The promoter may also be inducible by an exogenous agent. For example, a strong constitutive promoter could be used to cause increased levels of gene expression in both non-stress and stressed plants which in turn, results in enhanced freezing and dehydration tolerance. A tissue specific promoter could be used to alter gene expression in tissues that are highly sensitive to stress (and thereby enhance the stress tolerance of these tissues). Examples of such strong constitutive promoters include, but are not limited to, the nopaline synthase (NOS) and octopine synthase (OCS) promoters, the cauliflower mosaic virus (CaMV) 19S and 35S (Odell et al., Nature 313: 810-812 (1985)) promoters or the enhanced CaMV 35S promoters (Kay et al., Science 236: 1299-1302 (1987)).

A tissue-specific promoter could also be used to alter gene expression in tissues that are highly sensitive to stress, thereby enhancing the stress tolerance of these tissues. Examples of tissue-specific promoters include, but are not limited to, seed-specific promoters for the B. napus napin gene (U.S. Pat. No. 5,420,034), the soybean 7S promoter, the Arabidopsis 12S globulin (cruiferin) promoter (Pang, et al. Plant Molecular Biology 11: 805-820 (1988)), the maize 27 kd zein promoter, the rice glutelin 1 promoter and the phytohemaglutinin gene, fruit active promoters such as the E8 promoter from tomatoes, tuber-specific promoters such as the patatin promoter, and the promoter for the small subunit of ribuloe-1,5-bis-phosphate carboxylase (ssRUBISCO) whose expression is activated in photosynthetic tissues such as leaves.

Alternatively, an inducible promoter may be used to control the expression of the regulatory binding protein, such as RAV1 (Seq. ID No. 1, FIG. 11) or ZAT 12 (Seq. ID No. 2, FIG. 12), in plants. Because, in some cases, constitutive expression of higher levels of RAV1 or ZAT12 proteins may have some detrimental effects on plant growth and development, the controlled expression of RAV1 or ZAT12 genes is especially advantageous. For example, a promoter could be used to induce the expression of RAV1 or ZAT12 proteins only at a proper time, such as prior to a frost that may occur earlier or later in the growing season of a plant, thereby prolonging the growing season of a crop and increasing the productivity of the land. This may be accomplished by applying an exogenous inducer by a grower whenever desired. Alternatively, a promoter could be used which turns on at a temperature that is warmer than the temperature at which the plant normally exhibits cold tolerance. This would enable the cold tolerance thermostat of a plant to be altered. Similarly, a promoter can be used that turns on at a dehydration condition that is wetter than the dehydration condition at which the plant normally exhibits dehydration tolerance. This would enable the level at which a plant responds to dehydration to be altered.

Promoters, which are known, or are found to cause inducible transcription of the DNA into mRNA in plant cells may be used in the present invention. Such promoters may be obtained from a variety of sources such as plant and inducible microbial sources and may be activated by a variety of exogenous stimuli such as cold, heat, dehydration, pathogenesis and chemical treatment. The particular promoter selected is preferably capable of causing sufficient expression of the regulatory binding protein, such as RAV1 or ZAT12, to enhance plant tolerance to environmental stresses. Examples of promoters that may be used include, but are not limited to, the promoter for the DRE (C-repeat) binding protein gene dreb2a (Liu, et al. Plant Cell 10: 1391-1406 (1998)) that is activated by dehydration and high-salt stress, the promoter for delta 1-pyrroline-5-carboxylate synthetase (P5CS) whose expression is induced by dehydration, high salt and treatment with plant hormone abscisic acid (ABA) (Yoshiba, et al., Plant J. 7 751-760 (1987)), the promoters for the rd22 gene from Arabidopsis whose transcription is induced under by salt stress, water deficit and endogenous ABA (Yamaguchi-Shinozaki and Shinozaki, Mol Gen Genet 238 17-25 (1993)), the promoter for the rd29b gene (Yamaguchi-Shinizaki and Shinozaki, Plant Physiol., 101 1119-1120 (1993)) whose expression is induced by desiccation, salt stress and exogenous ABA treatment (Ishitani et al., Plant Cell 10 1151-1161 (1998)), the promoter for the rab1 8 gene from Arabidopsis whose transcripts accumulate in plants exposed to water deficit or exogenous ABA treatment, and the promoter for the pathogenesis-related protein 1a (PR-1a) gene whose expression is induced by pathogenesis organisms or by chemicals such as salicylic acid and polyacrylic acid.

It should be noted that the promoters described above may be further modified to alter their expression characteristics. For example, the drought/ABA inducible promoter for the rab18 gene may be incorporated into seed-specific promoters such that the rab 18 promoter is drought/ABA inducible only when developing seeds. Similarly, any number of chimeric promoters may be created by ligating a DNA fragment sufficient to confer environmental stress inducibility from the promoters described above to constitute promoters with other specificities such as tissue-specific promoters, developmentally regulated promoters, light-regulated promoters, hormone-responsive promoters, and the like. This should result in the creation of chimeric promoters capable of being used to cause expression of the regulatory binding proteins in any plant tissue or combination of plant tissues. Expression may also be induced either at a specific time during a plant's life cycle or throughout the plant's life cycle.

According to the present invention, an expression vector may be constructed to express the regulatory binding protein in the transformed plants to enhance their tolerance to environmental stresses. In one embodiment, the DNA construct may contain: (1) an inducible promoter that activates expression of the regulatory binding protein in response to environmental stimuli; (2) a sequence encoding the regulatory binding protein; and (3) a 3′ non-translated region which enables 3′ transcriptional termination and polyadenylation of the mRNA transcript. The inducible promoter may be any one of the natural or recombinant promoters described above. The gene encoding the regulatory binding protein may be any one disclosed in the present invention. The 3′ region downstream from this gene should be capable of providing a polyadenylation signal and other regulatory sequences that may be required for the proper expression and processing of a mRNA may be operably linked to the 3′ end of a structural gene to accomplish the invention. This may include the native 3′ end of the homologous gene form which the regulatory binding protein and/or the inducible promoter is derived, the 3′ end from a heterologous gene encoding the same protein from other species, the 3′ end from viral genes such as the 3′ end of the 35S or the 19S cauliflower mosaic virus transcripts, the 3′ end of the opine synthesis genes of Agrobacterium tumefaciens, or the 3′ end sequences from any source such that the sequence employed provides the necessary regulatory information within its nucleic acid sequence to result in the proper expression of the promoter/coding region combination to which the 3′ end sequence is operably linked.

A variety of expression vectors may be used to transfer the gene encoding the regulatory binding protein as well as the desired promoter into the plant. Examples include but are not limited to those derived from a Ti plasmid of Agrobacterium tumefaciens, as well as those disclosed by Herrera-Estrella, L., et al., Nature 303: 209(1983), Bevan, M., Nucl. Acids Res. 12: 8711-8721 (1984), Klee, H. J., Bio/Technology 3: 637-642 (1985), and EPO Publication 120,516 (Schilperoort et al.) for dicotyledonous plants. Alternatively, non-Ti vectors can be used to transfer the DNA constructs of this invention into monotyledonous plants and plant cells by using free DNA delivery techniques. Such methods may involve, for example, the use of liposomes, electroporation, microprojectile bombardment, silicon carbide wiskers, viruses and pollen. By using these methods transgenic plants such as wheat, rice (Christou, P., Bio/Technology 9: 957-962 (1991)) and corn (Gordon-Kamm, W., Plant Cell 2: 603-618 (1990)) are produced. An immature embryo may also be a good target tissue for monocots for direct DNA delivery techniques by using a particle gun (Weeks, T. et al., Plant Physiol. 102: 1077-1084 (1993); Vasil, V., Bio/Technology 10: 667-674 (1993); Wan, Y. and Lemeaux, P., Plant Physiol. 104: 37-48 (1994), and for Agrobacterium-mediated DNA transfer (Hiei et al., Plant J. 6: 271-282 (1994); Rashid et al., Plant Cell Rep. 15: 727-730 (1996); Dong, J., et al., Mol. Breeding 2: 267-276 (1996); Aldemita, R. and Hodges, T., Planta 199: 612-617 (1996); Ishida et al., Nature Biotech. 14: 745-750 (1996)).

In one embodiment, the plasmid vector pMEN020 is preferred, which is derived from a Ti plasmid pMON10098, which is the type of binary vector described in U.S. Pat. Nos. 5,773,701 and 5,773,696. It is noted that other similar plasmids are possible to practice the present invention and the plasmid described is for illustrative purposes only. PMEN20 differs from pMON10098 by the substitution of a Kpnl, Sall, Sacl, Sacll, Notl, and XbaI restriction sites between the ECaMV 35S promoter and the E9 3′ region. Plasmid pMON10098 contains the following DNA segments. Starting at the bottom of the plasmid map is the origin of bacterial replication for maintenance in E. coli (ori-322). Moving in a counter-clockwise direction on the map, next is ori-V, which is the vegetative origin of replication (Stalker et al. Mol. Gen. Genet. 181:8-12 (1981)). Next is the left border of the T-DNA. Next is the chimeric gene used as the selectable marker. The chimera includes the 0.35 kilobase (kb) of the cauliflower mosaic virus 35S promoter (P-³⁵S) (Odell et al. (1985) Nature 313:810-812). A 0.84 kb neomycin phosphotransferase type 11 gene (KAN) and a 0.25 kb 31 non-translated region of the nopaline synthase gene (NOS 31) (Fraley et al. (1983) Proc. Natl. Acad. Sci. USA 80:1803-1807). The next sequence contains the enhanced CaMV 35S promoter and E9 3′ region gene cassette and restriction sites for inserting genes such as the coding region of RAV1 or ZAT12 genes. This chimeric gene cassette ends with the 0.65 kb of the E9 3′ region from the pea small subunit of RUBISCO gene (U.S. Pat. No. 5,773,701). Next is the right border of the T-DNA. Next is the 0.93 kb fragment isolated from transposon Tn7 that encodes the bacterial spectinomycin/streptomycin resistance (Spc/Str), which is a determinant for selection in E. coli and Agrobacterium tumefaciens (Fling et al., Nucl. Acids Res. 13:7095-7106 (1985)).

The pMEN020 plasmid construct is a binary cloning vector that contains both E. coli and Agrobacterium tumefaciens origins of DNA replication but no vir genes encoding proteins essential for the transfer and integration of the target gene inserted in the T-DNA region. PMEN020 requires the trfA gene product to replicate in Agrobacterium. The strain of Agrobacterium containing this trfA gene is called the ABI strain and is described below and in U.S. Pat. Nos. 5,773,701 and 5,773,696. This cloning vector serves as an E. coli Agrobacterium tumefaciens shuttle vector. All of the cloning steps are carried out in E. coli before the vector is introduced into ABI strain of Agribacterium tumefaciens.

The recipient ABI strain of Agribacterium carries a modified defective Ti plasmid that serves as a helper plasmid containing a complete set of vir genes but lacks portions or all of the T-DNA region. ABI is the A208 Agrobacterium tumefaciens strain carrying the disarmed pTiC58 plasmid pMP90RK (Koncz et al. Mol. Gen. Genet. 204:383-396 (1986)). The disarmed Ti plasmid provides the trfA gene functions that are required for autonomous replication of the binary vectors after transfer into the ABI strain. When plant tissue is incubated with the ABI::binary vector strains, the vectors are transferred to the plant cells by the vir functions encoded by the disarmed pMP90RK Ti plasmid. After the introduction of the binary vector into the recipient Agribacterium, the vir gene products mobilize the T-DNA region of the pMEN020 plasmid to insert the target gene, e.g. the gene encoding the regulatory binding protein, into the plant chromosomal DNA, thus transforming the cell.

After transformation of cells or protoplasts, the choice of methods for regenerating fertile plants is not a critical element of the present invention. Suitable protocols exist in the art for Leguminosae (alfalfa, soybean, clover, etc.), Umbelliferae (Carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops (See protocols described in Ammirato et al. (1984) Handbook of Plant Cell Culture-Crop Species. Macmillan Publ. Co.; Shimamoto et al. Nature 338:274-276 (1989); Fromm et al., Bio/Technology 8:833-839 (1990); and Vasil et al. Bio/Technology 8:429-434 (1990).

It is envisioned that the present invention may be used to introduce, change and/or augment the environmental stress tolerance of a plant by introducing and causing the expression of environmental stress tolerance in a manner which the plant does not exhibit in its native form. For example, by using different promoters in combination with recombinant regulatory genes, native environmental stress tolerance genes can be expressed independent of environmental stress, made responsive to different levels or types of environmental stress, or rendered inducible independent of an environmental stress. Further, selection of the promoter may also be used to determine what tissues in the plant express the binding protein as well as when the expression occurs in the plant's lifecycle. By selecting a promoter which regulates in what tissues and when in a plants life the promoter functions to regulate expression of the binding protein, in combination with the selecting how that promoter regulates expression (level of expression and/or type of environmental or chemical induction), an incredible range of control over the environmental stress responses of a plant can be achieved using the present invention.

By recombinantly introducing a native environmental stress tolerance gene into a plant in combination with a recombinant regulatory gene under the control of an inducible promoter, a plant can be engineered which includes its native environmental stress tolerance as well as inducible environmental stress tolerance. This might be useful for inducing a cold stress tolerance reaction in anticipation of a frost.

By recombinantly introducing a non-native environmental stress tolerance gene into a plant in combination with a recombinant regulatory gene, a plant may be engineered that includes environmental stress tolerance properties that the plant would not otherwise have. In this regard, plants from warmer climates may be engineered to include one or more cold tolerance genes along with a regulatory gene needed to cause expression of the cold tolerance genes in the plant so that the engineered plant can survive better in a colder climate. Similarly, a plant may be engineered to include one or more dehydration tolerance genes along with a regulatory gene needed to cause expression of the dehydration tolerance gene so that the engineered plant can grow better in a dryer climate. In this regard, it should be possible to take a plant which grows well in a first climate and engineer it to include stress tolerance genes and regulatory genes native to a second climate so that the plant can grow well in the second climate.

By modifying the promoter controlling the expression of the gene encoding a binding protein that regulates the expression of environmental stress tolerance genes, the operation of native, non-recombinant environmental stress tolerance genes and regulatory genes may be changed. For example, the conditions under which the stress tolerance genes are expressed can be changed. Expression may also be rendered inducible by an exogenous agent.

The examples and other embodiments described herein are exemplary and not intended to be limiting in describing the full scope of sequences and methods of this invention. Equivalent changes, modifications and variations of specific embodiments, materials, plant species, compositions, homologous sequences, and methods may be made within the scope of the present invention, with substantially similar results. While the present invention is disclosed by reference to the preferred embodiments and examples detailed above, it is to be understood that these examples are intended in an illustrative rather than limiting sense, as it is contemplated that modifications will readily occur to those skilled in the art, which modifications will be within the spirit of the invention and the scope of the appended claims. All patents and cited articles are incorporated by reference

1. Plant material transformed with DNA encoding a binding protein comprising an AP2 domain amino acid sequence as set forth in SEQ. ID. No. 1.

2. Plant material transformed with DNA encoding a transcription regulating protein from the group comprising SEQ. ID No. 1 and SEQ. ID. No. 2.

3. A chimeric plant-expressible gene, said gene comprising in the 5′ to 3′ direction:
(a) a promoter capable of effecting mRNA transcription in the selected plant cell to be transformed, operably linked to
(b) a structural DNA sequence encoding SEQ. ID. No. 1 that induces freezing tolerance, operably linked to
(c) a non-translated region of a gene, said region encoding a signal sequence for polyadenylation of mRNA.

4. A chimeric plant-expressible gene, said gene comprising in the 5′ to 3′ direction:
(a) a promoter that is capable of effecting mRNA transcription in the selected plant cell to be transformed, operably linked to
(b) a structural DNA sequence encoding SEQ. ID. No. 2 that induces freezing tolerance, operably linked to
(c) a non-translated region of a gene, said region encoding a signal sequence for polyadenylation of mRNA.

5. A chimeric plant-expressible gene, said gene comprising in the 5′ to 3′ direction:
(a) a promoter that is capable of effecting mRNA transcription in the selected plant cell to be transformed, operably linked to
(b) a structural DNA sequence encoding SEQ. ID. No. 1 that induces drought tolerance, operably linked to
(c) a non-translated region of a gene, said region encoding a signal sequence for polyadenylation of mRNA.

6. A chimeric plant-expressible gene, said gene comprising in the 5′ to 3′ direction:
(a) a promoter that is capable of effecting mRNA transcription in the selected plant cell to be transformed, operably linked to
(b) a structural DNA sequence encoding essentially SEQ. ID. No. 2 that induces drought tolerance, operably linked to
(c) a non-translated region of a gene said region encoding a signal sequence for polyadenylation of mRNA.

7. A chimeric gene capable of expressing a polypeptide in plant cells comprising in sequence:
(a) a promoter;
(b) a 5′ non-translated region;
(c) a structural coding sequence encoding SEQ. ID No. 1; and
(d) a 3′ non-translated region of a gene.

8. Plant tissue comprising plant cells susceptible to infection with Agrobacierieim tumefaciens that contain and express a chimeric gene of claim 4, 5, 6, or 7.

9. A method for regulating cold and dehydration regulatory genes in a plant comprising the steps of:
introducing at least one copy of a regulatory gene encoding a protein into a plant;
expressing the binding protein encoded by the regulatory gene; and
using the expressed binding protein to stimulate expression of at least one environmental stress tolerance gene through binding to a DNA regulatory sequence.

10. A method for regulating cold and dehydration regulatory genes in a plant comprising the steps of:
transforming a plant with a gene encoding a transcription regulating protein comprising an amino acid sequence sufficiently homologous to SEQ. ID. No. 2 that the protein is capable of selectively binding to a DNA regulatory sequence in the plant which regulates expression of one or more environmental stress tolerance genes in the plant; and
expressing the transcription regulating protein in the plant.

11. A method for regulating cold and dehydration regulatory genes in a plant comprising the steps of:
introducing DNA encoding a binding protein capable of binding to a DNA regulatory sequence into a plant;
introducing a promoter into a plant which regulates expression of the binding protein;
introducing a DNA regulatory sequence into a plant to which a binding protein can bind; and
introducing one or more environmental stress tolerance genes into a plant whose expression is regulated by a DNA regulatory sequence.

12. A method for regulating cold and dehydration regulatory genes in a plant comprising the steps of:
transforming a plant with a gene encoding a transcription regulating protein comprising an amino acid sequence sufficiently homologous to SEQ. ID. No. 1 that the protein is capable of selectively binding to a DNA regulatory sequence comprising CAACA in the plant which regulates expression of one or more environmental stress tolerance genes in the plant; and
expressing the transcription regulating protein in the plant.

13. A chimeric plant-expressible gene, said gene comprising in the 5′ to 3′ direction:
(a) a promoter that is capable of effecting mRNA transcription in the selected plant cell to be transformed, operably linked to
(b) a structural DNA sequence encoding SEQ. ID. No. 2 that induces freezing tolerance.

14. A chimeric plant-expressible gene, said gene comprising in the 5′ to 3′ direction:
(a) a promoter that is capable of effecting mRNA transcription in the selected plant cell to be transformed, operably linked to
(b) a structural DNA sequence encoding SEQ. ID. No. 1 that induces drought tolerance.

15. Plant material transformed with DNA encoding a transcription regulating protein from an amino acid sequence that is at least 85% homologous to SEQ. ID No. 1.

16. Plant material transformed with DNA encoding a transcription regulating protein from an amino acid sequence that substantially similar to SEQ. ID No. 1.

17. Plant material transformed with DNA encoding a cold-regulated transcription factor.

18. The plant material of claim 17, wherein the cold-regulated transcription factor is ZAT12.

19. The plant material of claim 17, wherein the cold-regulated transcription factor is RAV1.
